SEQUENCE LISTING (66 sequences)

SEQ ID NO: 1 - DNA, 23 bp, Artificial Sequence
  Synthetic
catgttccagtatgactccactc

SEQ ID NO: 2 - DNA, 20 bp, Artificial Sequence
  Synthetic
tgtagtttaccccactccgg

SEQ ID NO: 3 - DNA, 20 bp, Artificial Sequence
  Synthetic
gggcagaaagattcaagacc

SEQ ID NO: 4 - DNA, 18 bp, Artificial Sequence
  Synthetic
cctacggttacagaagcg

SEQ ID NO: 5 - DNA, 20 bp, Artificial Sequence
  Synthetic
caaacggagtgcaatcaaga

SEQ ID NO: 6 - DNA, 20 bp, Artificial Sequence
  Synthetic
atagccagaggtctccacga

SEQ ID NO: 7 - DNA, 20 bp, Artificial Sequence
  Synthetic
catcaagaggatcagggagc

SEQ ID NO: 8 - DNA, 23 bp, Artificial Sequence
  Synthetic
ctttgactttcgacttctgttgg

SEQ ID NO: 9 - DNA, 19 bp, Artificial Sequence
  Synthetic
agccaatcccaaccagaac

SEQ ID NO: 10 - DNA, 21 bp, Artificial Sequence
  Synthetic
gtactcaccctaaagaccaca

SEQ ID NO: 11 - DNA, 23 bp, Artificial Sequence
  Synthetic
ctatcccagaggctttatccaag

SEQ ID NO: 12 - DNA, 22 bp, Artificial Sequence
  Synthetic
cgatggcaagaaaagtgagacc

SEQ ID NO: 13 - DNA, 21 bp, Artificial Sequence
  Synthetic
gccaatcagactacaccaagg

SEQ ID NO: 14 - DNA, 20 bp, Artificial Sequence
  Synthetic
gttcttcgttgtgttcaccg

SEQ ID NO: 15 - DNA, 20 bp, Artificial Sequence
  Synthetic
ccatctgcccaatatccctg

SEQ ID NO: 16 - DNA, 20 bp, Artificial Sequence
  Synthetic
ccgagtcaaactctacctgt

SEQ ID NO: 17 - DNA, 19 bp, Artificial Sequence
  Synthetic
aagttgtcggagctggatg

SEQ ID NO: 18 - DNA, 20 bp, Artificial Sequence
  Synthetic
gtagaaccctcactagacgc

SEQ ID NO: 19 - protein, 802 aa, Homo sapiens
MSEQSICQARAAVMVYDDANKKWVPAGGSTGFSRVHIYHHTGNNTFRVVGRKIQDHQVVINCAIPKGLKYNQATQTFHQWRDARQVYGLNFGSKEDANVFASAMMHALEVLNSQEAAQSKVTATQDSTNLRCIFCGPTLPRQNSQLPAQVQNGPSQEELEIQRRQLQEQQRQKELERERMERERLERERLERERLERERLEQEQLERQRQEREHVERLERERLERLERERQERERERLEQLEREQVEWERERRMSNAAPSSDSSLSSAPLPEYSSCQPPSAPPPSYAKVISAPVSDATPDYAVVTALPPTSTPPTPPLRHAATRFATSLGSAFHPVLPHYATVPRPLNKNSRPSSPVNTPSSQPPAAKSCAWPTSNFSPLPPSPPIMISSPPGKATGPRPVLPVCVSSPVPQMPPSPTAPNGSLDSVTYPVSPPPTSGPAAPPPPPPPPPPPPPPPLPPPPLPPLASLSHCGSQASPPPGTPLASTPSSKPSVLPSPSAGAPASAETPLNPELGDSSASEPGLQAASQPAESPTPQGLVLGPPAPPPPPPLPSGPAYASALPPPPGPPPPPPLPSTGPPPPPPPPPPLPNQAPPPPPPPPAPPLPASGIFSGSTSEDNRPLTGLAAAIAGAKLRKVSRVEDGSFPGGGNTGSVSLASSKADAGRGNGPLPLGGSGLMEEMSALLARRRRIAEKGSTIETEQKEDRNEDAEPITAKAPSTSTPEPTRKPWERTNTMNGSKSPVISRPKSTPSSQPSANGVQTEGLDYDRLKQDILDEMRKELAKLKEELIDAIRQELSKSNTA

SEQ ID NO: 20 - protein, 591 aa, Homo sapiens
MSEQSICQARAAVMVYDDANKKWVPAGGSTGFSRVHIYHHTGNNTFRVVGRKIQDHQVVINCAIPKGLKYNQATQTFHQWRDARQVYGLNFGSKEDANVFASAMMHALEVLNSQETGPTLPRQNSQLPAQVQNGPSQEELEIQRRQLQEQQRQKELERERLERERMERERLERERLERERLERERLEQEQLERERQERERQERLERQERLERQERLERQERLDRERQERQERERLERLERERQERERQEQLEREQLEWERERRISSAAAPASVETPLNSVLGDSSASEPGLQAASQPAETPSQQGIVLGPLAPPPPPPLPPGPAQASVALPPPPGPPPPPPLPSTGPPPPPPPPPLPNQVPPPPPPPPAPPLPASGFFLASMSEDNRPLTGLAAAIAGAKLRKVSRMEDTSFPSGGNAIGVNSASSKTDTGRGNGPLPLGGSGLMEEMSALLARRRRIAEKGSTIETEQKEDKGEDSEPVTSKASSTSTPEPTRKPWERTNTMNGSKSPVISRRDSPRKNQIVFDNRSYDSLHRPKSTPLSQPSANGVQTEGLDYDRLKQDILDEMRKELTKLKEELIDAIRQELSKSNTA

SEQ ID NO: 21 - protein, 763 aa, Mus musculus
MHTGGETSACKPSSVRLAPSFSFHAAGLQMAAQMPHSHQYSDRRQPSISDQQVSALPYSDQIQQPLTNQVMPDIVMLQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWSLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGSSGTSNSGRARSDPTHQHRHSGGHFAAAVQAMDCETHSPQVRQQFPAPLGWSGTEAPTQVTVETHPVQETTFHVAPQQNALHHHHGNSSHHHHHHHHHHHHHGQQALGNRTRPRVYNSPTNSSSTQDSMEVGHSHHSMTSLSSSTTSSSTSSSSTGNQGNQAYQNRPVAANTLDFGQNGAMDVNLTVYSNPRQETGIAGHPTYQFSANTGPAHYMTEGHLAMRQGADREESPMTGVCVQQSPVASS

SEQ ID NO: 22 - protein, 763 aa, Homo sapiens
MHTGGETSACKPSSVRLAPSFSFHAAGLQMAGQMPHSHQYSDRRQPNISDQQVSALSYSDQIQQPLTNQVMPDIVMLQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGSSGTSNSGRARSDPTHQHRHSGGHFTAAVQAMDCETHSPQVRQQFPAPLGWSGTEAPTQVTVETHPVQETTFHVAPQQNALHHHHGNSSHHHHHHHHHHHHHGQQALGNRTRPRVYNSPTNSSSTQDSMEVGHSHHSMTSLSSSTTSSSTSSSSTGNQGNQAYQNRPVAANTLDFGQNGAMDVNLTVYSNPRQETGIAGHPTYQFSANTGPAHYMTEGHLTMRQGADREESPMTGVCVQQSPVASS

SEQ ID NO: 23 - protein, 463 aa, Mus musculus
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 24 - protein, 463 aa, Homo sapiens; identical to SEQ ID NO: 23

SEQ ID NO: 25 - protein, 356 aa, Mus musculus
MDAGVTESGLNVTLTIRLLMHGKEVGSIIGKKGESVKRIREESGARINISEGNCPERIITLTGPTNAIFKAFAMIIDKLEEDINSSMTNSTAASRPPVTLRLVVPATQCGSLIGKGGCKIKEIRESTGAQVQVAGDMLPNSTERAITIAGVPQSVTECVKQICLVMLETLSQSPQGRVMTIPYQPMPASSPVICAGGQDRCSDAAGYPHATHDLEGPPLDAYSIQGQHTISPLDLAKLNQVARQQSHFAMMHGGTGFAGIDSSSPEVKGYWASLDASTQTTHELTIPNNLIGCIIGRQGANINEIRQMSGAQIKIANPVEGSSGRQVTITGSAASISLAQYLINARLSSEKGMGCS

SEQ ID NO: 26 - protein, 356 aa, Homo sapiens; identical to SEQ ID NO: 25

SEQ ID NO: 27 - protein, 770 aa, Mus musculus
MLPSLALLLLAAWTVRALEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSVDSADAEEDDSDVWWGGADTDYADGGEDKVVEVAEEEEVADVEEEEADDDEDVEDGDEVEEEAEEPYEEATERTTSTATTTTTTTESVEEVVREVCSEQAETGPCRAMISRWYFDVTEGKCVPFFYGGCGGNRNNFDTEEYCMAVCGSVSTQSLLKTTSEPLPQDPDKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVKMDAEFGHDSGFEVRHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 28 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 29 - protein, 991 aa, Mus musculus
MAEPGTGSGDPAFGPGASESGTRRLSDLRVIDLRAELKKRNLDTGGNKSVLMERLRKAFKEEGQEPEEVGISWGAVSKRAVKRNTKGSKMEEEGSEDNGLEEDSRYGQDGVVILQSSQDRDTMDTGVPDGMEAEDLSVPCLGKADTVNQILHAFDDSKEYVAAQLGQLPAQLLKHAVDEEVFKNTLEASVSDLKVTLADEEAPMEPENEKILDILGETCKSEPVKEEGSELEQPFAQATSSVGPDRKLAEEEDLFESCGHPEEEEEEEEEDQEEEQEEEGDLALASSSKSESPSTRCQWSEADAPLAVVKRELADAPGGGGGTRHRRKRKRRRKHQAQAEALGTGGGAGMNCEPVGLEEPVEQSSTAAQLPEATSQELVRAPTAALSPEPQDSKEDVKKFAFDACNDVPAPPKESSASEGADQKMSSVKEEQDIKPVIKDEKGRASCSSGRNLWVSGLSSSTRAADLKSLFSKHGKVIGAKVVTNARSPGARCYGFVTMSTSDEATKCISHLHRTELHGRMISVEKAKNEPSEKKSSDRRACDQKEKVPGPDRPHPVKIKTEKTVIKKEEKLERKEEKGPEDIKKEKDQDELTPGAAGHSRVTKSGSRGMERTVVMDKSKGEPVISVKATSRSKDRSSKSQDRKSEGREKRDILSFDKIKEQRERERQRQREREIRETERRREREQREREQRLDAFQERREKARLQRERMQLQCQRQRLERERLERERLERERMRVERERRKEQQRIMREREELRRQQEQLRAEQERRALRRPYDLDARRDDGYWPEGKRAALEDRYRDFPRPDHRFHDFDHRDRGHYQEHVIDRRDGSRTRVEERDGQYYPDDQHSHGRLLEHHAWDSGDGWHGYSSDKKLNEGQGLPPPPRVSREWAEHSSQLEEQQVPVWHSAVDTNMTGHEHIRWRGAERGLAGGPGHGHVAAGRGGMAGQGSFAHGGHSQGYIVPSGRLEGGGMASQDQGGRVPNPHPHPHFTRRY

SEQ ID NO: 30 - protein, 953 aa, Homo sapiens
MAETLPGSGDSGPGTASLGPGVAETGTRRLSELRVIDLRAELKKRNLDTGGNKSVLMERLKKAVKEEGQDPDEIGIELEATSKKSAKRCVKGLKMEEEGTEDNGLEDDSRDGQEDMEASLENLQNMGMMDMSVLDETEVANSSAPDFGEDGTDGLLDSFCDSKEYVAAQLRQLPAQPPEHAVDGEGFKNTLETSSLNFKVTPDIEESLLEPENEKILDILGETCKSEPVKEESSELEQPFAQDTSSVGPDRKLAEEEDLFDSAHPEEGDLDLASESTAHAQSSKADSLLAVVKREPAEQPGDGERTDCEPVGLEPAVEQSSAASELAEASSEELAEAPTEAPSPEARDSKEDGRKFDFDACNEVPPAPKESSTSEGADQKMSSFKEEKDIKPIIKDEKGRVGSGSGRNLWVSGLSSTTRATDLKNLFSKYGKVVGAKVVTNARSPGARCYGFVTMSTSDEATKCISHLHRTELHGRMISVEKAKNEPAGKKLSDRKECEVKKEKLSSVDRHHSVEIKIEKTVIKKEEKIEKKEEKKPEDIKKEEKDQDELKPGPTNRSRVTKSGSRGMERTVVMDKSKGEPVISVKTTSRSKERSSKSQDRKSESKEKRDILSFDKIKEQRERERQRQREREIRETERRREREQREREQRLEAFHERKEKARLQRERLQLECQRQRLERERMERERLERERMRVERERRKEQERIHREREELRRQQEQLRYEQERRPGRRPYDLDRRDDAYWPEGKRVAMEDRYRADFPRPDHRFHDFDHRDRGQYQDHAIDRREGSRPMMGDHRDGQHYGDDRHGHGGPPERHGRDSRDGWGGYGSDKRLSEGRGLPPPPRGGRDWGEHNQRLEEHQARAWQGAMDAGAASREHARWQGGERGLSGPSGPGHMASRGGVAGRGGFAQGGHSQGHVVPGGGLEGGGVASQDRGSRVPHPHPHPPPYPHFTRRY

SEQ ID NO: 31 - protein, 781 aa, Mus musculus
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEALGYRQDDPSYRSFHSGGYGQDALGMDPMMEHEMGGHHPGADYPVDGLPDLGHAQDLMDGLPPGDSNQLAWFDTDL

SEQ ID NO: 32 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 33 - protein, 1476 aa, Mus musculus
MKSLLNAFTKKEVPFREAPAYSNRRRRPPNTLAAPRVLLRSNSDNNLNAGAPEWAVCSAATSHRSLSPQLLQQTPSKPDGATKSLGSYTPGPRSRSPSLNRLGGTAEDGKRTQPHWHVGSPFTPGANKDSLSTFEYPGPRRKLYSAVPGRLFVAVKPYQPQVDGEIPLHRGDRVKVLSIGEGGFWEGSARGHIGWFPAECVEEVQCKPRDSQAETRADRSKKLFRHYTVGSYDSFDAASDCIIEDKTVVLQKKDNEGFGFVLRGAKADTPIEEFTPTPAFPALQYLESVDEGGVAWQAGLRTGDFLIEVNNENVVKVGHRQVVNMIRQGGNHLILKVVTVTRNLDPDDTARKKAPPPPKRAPTTALTLRSKSMTAELEELGLSLVDKASVRKKKDKPEEIVPASKPSRTAENVAIESRVATIKQRPTSRCFPAASDVNSVYERQGIAVMTPTVPGSPKGPFLGLPRGTMRRQKSIDSRIFLSGITEEERQFLAPPMLKFTRSLSMPDTSEDIPPPPQSVPPSPPPPSPTTYNCPRSPTPRVYGTIKPAFNQNPVVAKVPPATRSDTVATMMREKGMFYRRELDRFSLDSEDVYSRSPAPQAAFRTKRGQMPENPYSEVGKIASKAVYVPAKPARRKGVLVKQSNVEDSPEKTCSIPIPTIIVKEPSTSSSGKSSQGSSMEIDPQATEPGQLRPDDSLTVSSPFAAAIAGAVRDREKRLEARRNSPAFLSTDLGDEDVGLGPPAPRMQASKFPEEGGFGDEDETEQPLLPTPGAAPRELENHFLGGGEAGAQGEAGGPLSSTSKAKGPESGPAAPLKSSSPAGPENYVHPLTGRLLDPSSPLALALSARDRAMQESQQGHKGEAPKADLNKPLYIDTKMRPSVESGFPPVTRQNTRGPLRRQETENKYETDLGKDRRADDKKNMLINIVDTAQQKSAGLLMVHTVDVPMAGPPLEEEEDREDGDTKPDHSPSTVPEGVPKTEGALQISAAPEPAVAPGRTIVAAGSVEEAVILPFRIPPPPLASVDLDEDFLFTEPLPPPLEFANSFDIPDDRAASVPALADLVKQKKNDTSQPPTLNSSQPANSTDSKKPAGISNCLPSSFLPPPESFDAVTDSGIEEVDSRSSSDHHLETTSTISTVSSISTLSSEGGESMDTCTVYADGQAFVVDKPPVPPKPKMKPIVHKSNALYQDTLPEEDTDGFVIPPPAPPPPPGSAQAGVAKVIQPRTSKLWGDVPEVKSPILSGPKANVISELNSILQQMNRGKSVKPGEGLELPVGAKSANLAPRSPEVMSTVSGTRSTTVTFTVRPGTSQPITLQSRPPDYESRTSGPRRAPSPVVSPTELSKEILPTPPPPSATAASPSPTLSDVFSLPSQSPAGDLFGLNPAGRSRSPSPSILQQPISNKPFTTKPVHLWTKPDVADWLESLNLGEHKETFMDNEIDGSHLPNLQKEDLIDLGVTRVGHRMNIERALKQLLDR

SEQ ID NO: 34 - protein, 1470 aa, Homo sapiens
MKSLLNAFTKKEVPFREAPAYSNRRRRPPNTLAAPRVLLRSNSDNNLNASAPDWAVCSTATSHRSLSPQLLQQMPSKPEGAAKTIGSYVPGPRSRSPSLNRLGGAGEDGKRPQPLWHVGSPFALGANKDSLSAFEYPGPKRKLYSAVPGRLFVAVKPYQPQVDGEIPLHRGDRVKVLSIGEGGFWEGSARGHIGWFPAECVEEVQCKPRDSQAETRADRSKKLFRHYTVGSYDSFDTSSDCIIEEKTVVLQKKDNEGFGFVLRGAKADTPIEEFTPTPAFPALQYLESVDEGGVAWQAGLRTGDFLIEVNNENVVKVGHRQVVNMIRQGGNHLVLKVVTVTRNLDPDDTARKKAPPPPKRAPTTALTLRSKSMTSELEELVDKASVRKKKDKPEEIVPASKPSRAAENMAVEPRVATIKQRPSSRCFPAGSDMNSVYERQGIAVMTPTVPGSPKAPFLGIPRGTMRRQKSIDSRIFLSGITEEERQFLAPPMLKFTRSLSMPDTSEDIPPPPQSVPPSPPPPSPTTYNCPKSPTPRVYGTIKPAFNQNSAAKVSPATRSDTVATMMREKGMYFRRELDRYSLDSEDLYSRNAGPQANFRNKRGQMPENPYSEVGKIASKAVYVPAKPARRKGMLVKQSNVEDSPEKTCSIPIPTIIVKEPSTSSSGKSSQGSSMEIDPQAPEPPSQLRPDESLTVSSPFAAAIAGAVRDREKRLEARRNSPAFLSTDLGDEDVGLGPPAPRTRPSMFPEEGDFADEDSAEQLSSPMPSATPREPENHFVGGAEASAPGEAGRPLNSTSKAQGPESSPAVPSASSGTAGPGNYVHPLTGRLLDPSSPLALALSARDRAMKESQQGPKGEAPKADLNKPLYIDTKMRPSLDAGFPTVTRQNTRGPLRRQETENKYETDLGRDRKGDDKKNMLIDIMDTSQQKSAGLLMVHTVDATKLDNALQEEDEKAEVEMKPDSSPSEVPEGVSETEGALQISAAPEPTTVPGRTIVAVGSMEEAVILPFRIPPPPLASVDLDEDFIFTEPLPPPLEFANSFDIPDDRAASVPALSDLVKQKKSDTPQSPSLNSSQPTNSADSKKPASLSNCLPASFLPPPESFDAVADSGIEEVDSRSSSDHHLETTSTISTVSSISTLSSEGGENVDTCTVYADGQAFMVDKPPVPPKPKMKPIIHKSNALYQDALVEEDVDSFVIPPPAPPPPPGSAQPGMAKVLQPRTSKLWGDVTEIKSPILSGPKANVISELNSILQQMNREKLAKPGEGLDSPMGAKSASLAPRSPEIMSTISGTRSTTVTFTVRPGTSQPITLQSRPPDYESRTSGTRRAPSPVVSPTEMNKETLPAPLSAATASPSPALSDVFSLPSQPPSGDLFGLNPAGRSRSPSPSILQQPISNKPFTTKPVHLWTKPDVADWLESLNLGEHKEAFMDNEIDGSHLPNLQKEDLIDLGVTRVGHRMNIERALKQLLDR

SEQ ID NO: 35 - protein, 403 aa, Mus musculus
MTAIIKEIVSRNKRRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHSQITKV

SEQ ID NO: 36 - protein, 403 aa, Homo sapiens [sequence omitted]

SEQ ID NO: 37 - protein, 116 aa, Mus musculus
MSATAATVPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 38 - protein, 116 aa, Homo sapiens
MSATAATAPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 39 - protein, 326 aa, Mus musculus
MSNGYEDHMAEDCRDDIGRTNLIVNYLPQNMTQEELRSLFSSIGEVESAKLIRDKVAGHSLGYGFVNYVTAKDAERAISTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNMALLSQLYHSPARRFGGPVHHQAQRFRFSPMGVDHMSGISGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 40 - protein, 326 aa, Homo sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 41 - protein, 1612 aa, Mus musculus
MIAEPAHFYLFGLICLCSGSRLRQEDFPPRIVEHPSDLIVSKGEPATLNCKAEGRPTPTIEWYKGGERVETDKDDPRSHRMLLPSGSLFFLRIVHGRKSRPDEGVYICVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVKRPSNLAVTVDDSAEFKCEARGDPVPTVRWRKDDGELPKSRYEIRDDHTLKIRKVTAGDMGSYTCVAENMVGKAEASATLTVQEPPHFVVKPRDQVVALGRTVTFQCEATGNPQPAIFWRREGSQNLLFSYQPPQSSSRFSVSQTGDLTITNVQRSDVGYYICQTLNVAGSIITKAYLEVTDVIADRPPPVIRQGPVNQTVAVDGTLILSCVATGSPAPTILWRKDGVLVSTQDSRIKQLESGVLQIRYAKLGDTGRYTCTASTPSGEATWSAYIEVQEFGVPVQPPRPTDPNLIPSAPSKPEVTDVSKNTVTLSWQPNLNSGATPTSYIIEAFSHASGSSWQTAAENVKTETFAIKGLKPNAIYLFLVRAANAYGISDPSQISDPVKTQDVPPTSQGVDHKQVQRELGNVVLHLHNPTILSSSSVEVHWTVDQQSQYIQGYKILYRPSGASHGESEWLVFEVRTPTKNSVVIPDLRKGVNYEIKARPFFNEFQGADSEIKFAKTLEEAPSAPPRSVTVSKNDGNGTAILVTWQPPPEDTQNGMVQEYKVWCLGNETKYHINKTVDGSTFSVVIPSLVPGIRYSVEVAASTGAGPGVKSEPQFIQLDSHGNPVSPEDQVSLAQQISDVVRQPAFIAGIGAACWIILMVFSIWLYRHRKKRNGLTSTYAGIRKVPSFTFTPTVTYQRGGEAVSSGGRPGLLNISEPATQPWLADTWPNTGNNHNDCSINCCTAGNGNSDSNLTTYSRPADCIANYNNQLDNKQTNLMLPESTVYGDVDLSNKINEMKTFNSPNLKDGRFVNPSGQPTPYATTQLIQANLSNNMNNGAGDSSEKHWKPPGQQKPEVAPIQYNIMEQNKLNKDYRANDTIPPTIPYNQSYDQNTGGSYNSSDRGSSTSGSQGHKKGARTPKAPKQGGMNWADLLPPPPAHPPPHSNSEEYNMSVDESYDQEMPCPVPPAPMYLQQDELQEEEDERGPTPPVRGAASSPAAVSYSHQSTATLTPSPQEELQPMLQDCPEDLGHMPHPPDRRRQPVSPPPPPRPISPPHTYGYISGPLVSDMDTDAPEEEEDEADMEVAKMQTRRLLLRGLEQTPASSVGDLESSVTGSMINGWGSASEEDNISSGRSSVSSSDGSFFTDADFAQAVAAAAEYAGLKVARRQMQDAAGRRHFHASQCPRPTSPVSTDSNMSAVVIQKARPAKKQKHQPGHLRREAYADDLPPPPVPPPAIKSPTVQSKAQLEVRPVMVPKLASIEARTDRSSDRKGGSYKGREALDGRQVTDLRTNPSDPREAQEQPNDGKGRGTRQPKRDLPPAKTHLGQEDILPYCRPTFPTSNNPRDPSSSSSMSSRGSGSRQREQANVGRRNMAEMQVLGGFERGDENNEELEETES

SEQ ID NO: 42 - protein, 1651 aa, Homo sapiens
MKWKHVPFLVMISLLSLSPNHLFLAQLIPDPEDVERGNDHGTPIPTSDNDDNSLGYTGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVKRPSNLAVTVDDSAEFKCEARGDPVPTVRWRKDDGELPKSRYEIRDDHTLKIRKVTAGDMGSYTCVAENMVGKAEASATLTVQEPPHFVVKPRDQVVALGRTVTFQCEATGNPQPAIFWRREGSQNLLFSYQPPQSSSRFSVSQTGDLTITNVQRSDVGYYICQTLNVAGSIITKAYLEVTDVIADRPPPVIRQGPVNQTVAVDGTFVLSCVATGSPVPTILWRKDGVLVSTQDSRIKQLENGVLQIRYAKLGDTGRYTCIASTPSGEATWSAYIEVQEFGVPVQPPRPTDPNLIPSAPSKPEVTDVSRNTVTLSWQPNLNSGATPTSYIIEAFSHASGSSWQTVAENVKTETSAIKGLKPNAIYLFLVRAANAYGISDPSQISDPVKTQDVLPTSQGVDHKQVQRELGNAVLHLHNPTVLSSSSIEVHWTVDQQSQYIQGYKILYRPSGANHGESDWLVFEVRTPAKNSVVIPDLRKGVNYEIKARPFFNEFQGADSEIKFAKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLAQQISDVVKQPAFIAGIGAACWIILMVFSIWLYRHRKKRNGLTSTYAGIRKVPSFTFTPTVTYQRGGEAVSSGGRPGLLNISEPAAQPWLADTWPNTGNNHNDCSISCCTAGNGNSDSNLTTYSRPADCIANYNNQLDNKQTNLMLPESTVYGDVDLSNKINEMKTFNSPNLKDGRFVNPSGQPTPYATTQLIQSNLSNNMNNGSGDSGEKHWKPLGQQKQEVAPVQYNIVEQNKLNKDYRANDTVPPTIPYNQSYDQNTGGSYNSSDRGSSTSGSQGHKKGARTPKVPKQGGMNWADLLPPPPAHPPPHSNSEEYNISVDESYDQEMPCPVPPARMYLQQDELEEEEDERGPTPPVRGAASSPAAVSYSHQSTATLTPSPQEELQPMLQDCPEETGHMQHQPDRRRQPVSPPPPPRPISPPHTYGYISGPLVSDMDTDAPEEEEDEADMEVAKMQTRRLLLRGLEQTPASSVGDLESSVTGSMINGWGSASEEDNISSGRSSVSSSDGSFFTDADFAQAVAAAAEYAGLKVARRQMQDAAGRRHFHASQCPRPTSPVSTDSNMSAAVMQKTRPAKKLKHQPGHLRRETYTDDLPPPPVPPPAIKSPTAQSKTQLEVRPVVVPKLPSMDARTDRSSDRKGSSYKGREVLDGRQVVDMRTNPGDPREAQEQQNDGKGRGNKAAKRDLPPAKTHLIQEDILPYCRPTFPTSNNPRDPSSSSSMSSRGSGSRQREQANVGRRNIAEMQVLGGYERGEDNNEELEETES

SEQ ID NO: 43 - DNA, 2409 bp, Mus musculus
  misc_feature (1191)..(1191): n is a, c, g, t or u
atgagtgaacagagtatctgtcaggcaagagctgctgtgatggtctatgatgatgccaataagaagtgggtgccagctggtggctcaactgggttcagcagagtacatatatatcaccatacaggcaacaacacattcagagttgtgggcagaaagattcaagaccatcaggttgtgataaactgtgccattcctaaagggctgaagtacaatcaagctacacagactttccaccaatggagggatgctagacaggtgtatggtctcaactttggcagcaaagaggatgccaatgtcttcgcaagtgccatgatgcatgccttagaagtgttaaattcacaggaagcagcccagagcaaggttactgctacgcaggacagcactaatttgcgatgtattttctgtgggccaacattgcctagacaaaattcacagctacctgctcaagttcaaaatggcccatcccaagaagagctggaaatccagagaaggcaactgcaagaacagcagcgacagaaggaactggagagggaaagaatggagagggaaaggttggagagagaacgactagaacgagagaggctagagagggagcgcctggaacaagagcagctggagcggcagcggcaggaaagggagcacgtggagcggctggagagggagaggctggagcgcctggagcgagagaggcaggagcgggagcgagagcgcctggagcagctggagcgggagcaagtggagtgggagcgagagcgcagaatgtccaatgctgctccatcttcagacagctccctgtctagtgctccacttcctgagtattccagttgccagccgccttcggcacctcctccatcatatgctaaagtcatctcagctccggtgtcagacgccactcctgattacgctgtagtgactgctttgccacctacttccacaccccctacaccaccactgagacacgcagcgacacgttttgcaacatctctaggttcagccttccaccctgttcttccccattacgctacagttcctcgtcctctcaacaaaaactctcgaccttcttctcctgtgaacacaccctcttctcagcctccagctgcgaagtcctgtgcctggcctacttccaatttctcgcccctccctccatctcctccaataatgattagcagcccccctggcaaagctactggnccacggcctgtccttcccgtttgtgtctcctctcctgtgccccaaatgcctccgtcaccaacagcacccaatgggtcgctagactctgtaacatacccagtgtctccaccgcctacctcagggccagcagcgccacctccgccgccaccgccaccgccgccgccaccaccaccgccgctgccaccgccgccgctgcctcccctcgcctcactctcacactgtggatcacaggcttctcctcctccaggcacccctcttgcctcaactccctcatccaagcccagtgttctcccttctccctctgcaggtgcccctgcctctgcggagacccctctaaatcctgagctgggagactcctctgcttccgagccaggcttgcaggcagcctctcagccggccgagtcgccaaccccacagggccttgtcttgggaccacctgcacctccgccaccaccccctctcccatcaggccctgcctacgcctcagcacttcctcctcccccaggaccccctccaccacctccactgccatccactggtcctcctcctccaccccctccaccaccccctcttcctaatcaagctcctccccctcctcccccacctcctgcccctcccctccccgcatctggaattttctctggatccacgtcagaagacaatcgccctttaactggacttgcagctgcaattgcgggagcaaaacttaggaaagtgtcccgggtggaggatggctctttcccaggtggagggaatactgggagtgtgagcttggcctcatccaaagcagacgctgggcgtgggaatggacctcttcctctagggggtagtggcttaatggaagaaatgagtgccctgctggccaggaggagaagaattgctgagaagggatcaacaatagaaacagaacaaaaggaagacagaaatgaagatgcagagcctataactgctaaggccccatcaacaagtacacctgaaccaaccagaaaaccttgggaaagaacaaacacaatgaacggcagtaagtcacctgtcatctccagacccaaatccacaccttcatcacagccaagtgccaatggagtccagacagaaggccttgactatgacaggctgaagcaggacattttagatgagatgagaaaagagctggcaaagctgaaggaggagcttattgacgcaatcaggcaggagctgagcaagtcgaacactgcataa

SEQ ID NO: 44 - DNA, 1776 bp, Homo sapiens
atgagtgaacagagtatctgtcaggcaagagctgctgtgatggtttatgatgatgccaataagaagtgggtgccagctggtggctcaactggattcagcagagttcatatctatcaccatacaggcaacaacacattcagagtggtgggcaggaagattcaggaccatcaggtcgtgataaactgtgccattcctaaagggttgaagtacaatcaagctacacagaccttccaccagtggcgagatgctagacaggtgtatggtctcaactttggcagcaaagaggatgccaatgtcttcgcaagtgccatgatgcatgccttagaagtgttaaattcacaggaaacagggccaacattgcctagacaaaactcacaactacctgctcaagttcaaaatggcccatcccaagaagaattggaaattcaaagaagacaactacaagaacagcaacggcaaaaggagctggagcgggaaaggctggagcgagaaagaatggaaagagaaaggttggagagagagaggttagaaagggaaaggctggagagggagcgactggaacaagaacagctggagagagagagacaagaacgggaacggcaggaacgcctggagcggcaggaacgcctggagcggcaggaacgcctggagcggcaggaacgcctggatcgggagaggcaagaaagacaagaacgagagaggctggagagactggaacgggagaggcaagaaagggagcgacaagagcagttagaaagggaacagctggaatgggagagagagcgcagaatatcaagtgctgctgcccctgcctctgttgagactcctctaaactctgtgctgggagactcttctgcttctgagccaggcttgcaggcagcctctcagccggccgagactccatcccaacagggcattgtcttgggaccacttgcacctccacctcctccaccactcccaccagggcctgcacaggcttcagtagccctccctcctcccccagggccccctccacctcctccactcccatccaccgggcctccaccgccccctcctccccctcctctccctaatcaagtaccccctcctcctccaccacctcctgccccacccctccctgcatctggattctttttggcatccatgtcagaagacaatcgccctttaactggacttgcagctgcaattgccggagcaaaacttaggaaagtgtcacggatggaggatacctctttcccaagtggagggaatgctattggtgtgaactccgcctcatctaaaacagatacaggccgtggaaatggaccccttcctttagggggtagtggtttaatggaagaaatgagtgccctgctggccaggaggagaagaattgctgaaaagggatcaacaatagaaacagaacaaaaagaggacaaaggtgaagattcagagcctgtaacttctaaggcctcttcaacaagtacacctgaaccaacaagaaaaccttgggaaagaacaaatacaatgaatggcagcaagtcacctgttatctccagacgggattctccaaggaaaaatcagattgtttttgacaacaggtcctatgattcattacacagaccaaaatccacacccttatcacagcccagtgccaatggagtccagacggaaggacttgactatgacaggctgaagcaggacattttagatgaaatgagaaaagaattaacaaagctaaaagaagagctcattgatgcaatcaggcaggaactgagcaagtcaaatactgcatag

SEQ ID NO: 45 - DNA, 2292 bp, Mus musculus
atgcatacaggaggagagacttcagcatgcaaaccttcatctgtccggcttgcaccgtcgttctcattccatgctgctggccttcagatggctgcacagatgccccactcacaccagtacagtgaccgtcgccagccgagcataagtgaccagcaggtgtctgccttaccatattctgaccagattcagcaacctctaactaaccaggtgatgcctgacattgtcatgttacagaggcggatgccccaaaccttccgtgatccagcaactgctcctctgagaaaactctctgtggacttgatcaaaacatacaagcatattaatgaggtttactatgcaaaaaagaagcgaagacaccaacagggccagggggacgattccagtcataagaaggagcggaaggtttacaatgatggttacgatgatgataactatgattatattgtaaaaaacggggaaaagtggatggatcggtatgaaatcgactccttaataggcaaaggttcatttggacaggttgtgaaagcttatgacagagtggagcaagaatgggtcgccattaaaatcatcaagaacaagaaagcgtttctgaatcaagcccagatagaagtgcggctgcttgagctcatgaacaaacacgacactgaaatgaagtactacatagtgcatttgaaacgccactttatgtttcgaaaccatctctgtttagtgtttgaaatgctgtcctataatctctatgatttgttgagaaacaccaacttccgaggggtctctttgaacctaacacgaaagtttgcgcaacagatgtgcacagcattgctttttcttgcgactccagaacttagtatcattcactgtgacttaaagcctgagaacatccttctgtgtaaccccaaacggagtgcaatcaagattgttgattttggcagctcttgtcagttggggcagaggatataccagtatattcagagtcgcttttatcggtctccagaggtgctactgggaatgccttatgaccttgctatcgacatgtggtcccttggatgtatcttggttgaaatgcacactggagagcctctgttcagtggtgccaatgaggtcgatcagatgaataaaatagtggaagtcttgggcatcccacctgctcatattcttgaccaagcaccgaaagcaagaaagttctttgagaagttgcccgatggcacttggagcttaaagaagaccaaagatggaaaacgggagtacaaaccaccaggaacccgtaaacttcataatattcttggagtagaaacaggaggacctggcgggcggcgtgctggggaatcgggtcatactgtagctgactacttgaagttcaaagacctcattttaaggatgcttgattatgaccccaaaactcggattcaaccttattatgccctgcagcacagttttttcaagaaaacagctgatgaaggtaccaacacaagtaacagtgtgtctaccagccctgcgatggagcagtctcagtcttcaggcaccacctccagcacctcctccagctcaggtggatcctcgggaacgagtaacagtgggagagccaggtcggatccgacgcaccagcatcgacacagcggtggacacttcgctgctgctgtccaggccatggactgtgagacacacagtccccaggtgcgccagcagtttccggctcctctgggatggtcaggcactgaagctcctacacaagtcactgttgaaactcatcctgttcaagagacaacctttcatgtagccccccagcagaacgcattgcatcatcaccatggaaacagttcccatcaccaccaccaccaccatcaccaccaccaccaccatggacagcaagccttgggtaaccggaccaggccaagggtctacaattctccaacaaatagctcctctacccaggattctatggaggttggccacagtcaccactccatgacatccctgtcttcctcaacaacttcttcctcgacatcttcctcctctactggtaatcaaggcaatcaggcctatcagaaccgcccagtggctgctaacaccttggactttggacagaatggagctatggacgttaatttgaccgtctactccaatccccgccaagagactggcatagctggacatccaacataccaattttctgctaatacaggtcctgcacattacatgactgaaggacatctggcgatgagacaaggggctgatagagaagagtctcccatgacaggagtttgtgtgcaacagagtcctgtagctagctcgtga

SEQ ID NO: 46 - DNA, 2289 bp, Homo sapiens
atgcatacaggaggagagacttcagcatgcaaaccttcatctgttcggcttgcaccgtcattttcattccatgctgctggccttcagatggctggacagatgccccattcacatcagtacagtgaccgtcgccagccaaacataagtgaccaacaggtttctgccttatcatattctgaccagattcagcaacctctaactaaccaggtgatgcctgatattgtcatgttacagaggcggatgccccaaaccttccgtgacccagcaactgctcccctgagaaaactttctgttgacttgatcaaaacatacaagcatattaatgaggtttactatgcaaaaaagaagcgaagacaccaacagggccagggagacgattctagtcataagaaggaacggaaggtttacaatgatggttatgatgatgataactatgattatattgtaaaaaacggagaaaagtggatggatcgttacgaaattgactccttgataggcaaaggttcctttggacaggttgtaaaggcatatgatcgtgtggagcaagaatgggttgccattaaaataataaagaacaagaaggcttttctgaatcaagcacagatagaagtgcgacttcttgagctcatgaacaaacatgacactgaaatgaaatactacatagtgcatttgaaacgccactttatgtttcgaaaccatctctgtttagtttttgaaatgctgtcctacaacctctatgacttgctgagaaacaccaatttccgaggggtctctttgaacctaacacgaaagtttgcgcaacagatgtgcactgcactgcttttccttgcgactccagaacttagtatcattcactgtgatctaaaacctgaaaatatccttctttgtaaccccaaacgcagtgcaatcaagatagttgactttggcagttcttgtcagttggggcagaggatataccagtatattcagagtcgcttttatcggtctccagaggtgctactgggaatgccttatgaccttgccattgatatgtggtccctcgggtgtattttggttgaaatgcacactggagaacctctgttcagtggtgccaatgaggtagatcagatgaataaaatagtggaagttctgggtattccacctgctcatattcttgaccaagcaccaaaagcaagaaagttctttgagaagttgccagatggcacttggaacttaaagaagaccaaagatggaaaacgggagtacaaaccaccaggaacccgtaaacttcataacattcttggagtggaaacaggaggacctggtgggcgacgtgctggggagtcaggtcatacggtcgctgactacttgaagttcaaagacctcattttaaggatgcttgattatgaccccaaaactcgaattcaaccttattatgctctgcagcacagtttcttcaagaaaacagctgatgaaggtacaaatacaagtaatagtgtatctacaagccccgccatggagcagtctcagtcttcgggcaccacctccagtacatcgtcaagctcaggtggctcatcggggacaagcaacagtgggagagcccggtcggatccgacgcaccagcatcggcacagtggtgggcacttcacagctgccgtgcaggccatggactgcgagacacacagtccccaggtgcgtcagcaatttcctgctcctcttggttggtcaggcactgaagctcctacacaggtcactgttgaaactcatcctgttcaagaaacaacctttcatgtagcccctcaacagaatgcattgcatcatcaccatggtaacagttcccatcaccatcaccaccaccaccaccatcaccaccaccatggacaacaagccttgggtaaccggaccaggccaagggtctacaattctccaacgaatagctcctctacccaagattctatggaggttggccacagtcaccactccatgacatccctgtcttcctcaacgacttcttcctcgacatcttcctcctctactggtaaccaaggcaatcaggcctaccagaatcgcccagtggctgctaataccttggactttggacagaatggagctatggacgttaatttgaccgtctactccaatccccgccaagagactggcatagctggacatccaacataccaattttctgctaatacaggtcctgcacattacatgactgaaggacatctgacaatgaggcaaggggctgatagagaagagtcccccatgacaggagtttgtgtgcaacagagtcctgtagctagctcg

SEQ ID NO: 47 - DNA, 1392 bp, Mus musculus
atggagaccgaacagccagaagaaaccttccccaacaccgaaaccaatggtgaatttggtaaacgccctgcagaagatatggaagaggagcaagcctttaaaagatctagaaatactgatgagatggttgaattgcgcattttgcttcagagcaagaatgctggagcagtgattggaaaaggaggcaagaatattaaggctctccgtacagactacaatgccagtgtttcagtcccagacagcagtggccccgagcgcatactgagtatcagtgctgatattgagacgattggagaaattctgaagaaaatcatccctaccttggaagagggcctgcagttgccatcacccactgcaaccagccagctcccgctcgaatctgatgctgtggaatgcttaaattaccaacattataaaggaagtgactttgattgcgagttgagactgttgattcatcagagtctggcaggaggaataattggtgttaaaggtgctaaaatcaaagaacttcgagaaaacactcagacaacaatcaagcttttccaggagtgctgccctcactctactgacagagttgttcttattggaggaaaacctgatagggttgtagaatgcatcaagatcatccttgaccttatatctgagtctcccatcaaaggacgtgcacaaccttatgatcccaacttttatgatgagacctatgattatggtggttttacaatgatgtttgatgaccgccgaggacgacctgtgggattccccatgaggggaagaggtggttttgacagaatgcctcctggtcggggtgggcgtcccatgcctccttctagaagagattatgatgatatgagccctcgtcgaggacctccaccaccaccacctggtcgaggtggccggggtggcagcagagcccggaatctgcctcttcctcctccaccaccacccagagggggagatctaatggcttatgacagaagaggaaggcctggagaccgctatgatggcatggttgggttcagtgctgatgaaacttgggattctgcaattgacacatggagcccatcagaatggcaaatggcttatgaaccacagggtggttctggatatgactattcttatgcagggggccgtggctcatatggtgatcttggcggacctattatcactacacaagtaactattcccaaagatttggctggatctattattggcaaaggtggtcagcggattaaacaaattcgtcatgaatctggagcatcaatcaaaattgatgaacctttagaaggatctgaagatcggatcattaccattacaggaacacaggaccagatacagaacgcacagtatttgctgcagaacagtgtgaagcagtattctggaaagtttttctaa

SEQ ID NO: 48 - DNA, 1392 bp, Homo sapiens
atggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagggcctgcagttgccatcacccactgcaaccagccagctcccgctcgaatctgatgctgtggaatgcttaaattaccaacactataaaggaagtgactttgactgcgagttgaggctgttgattcatcagagtctagcaggaggaattattggggtcaaaggtgctaaaatcaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtattctggaaagtttttctaa

SEQ ID NO: 49 - DNA, 1071 bp, Mus musculus
atggacgccggtgtgactgaaagcggactcaacgtgactctcaccattcggctgctgatgcacggaaaggaagtaggcagcatcatcgggaagaaaggggagtcggtgaagaggatccgcgaggagagcggcgcgcggatcaacatctcggaggggaactgcccggagaggatcatcacgctgactgggcctaccaatgccatctttaaggccttcgccatgatcatcgacaagctggaggaagatatcaacagctccatgaccaacagtacggcggccagcaggcccccggtcacacttcggctggtcgtgcccgccacccagtgtggctccctgatcggcaagggcggctgcaagatcaaggagatccgcgagagcaccggggcccaggtccaggtggcgggggatatgctgcccaactcgaccgagcgggctatcactatcgccggcgtgccgcagtccgtcaccgagtgtgtgaagcagatctgcctggtcatgctggagacgctctcccagtctccacaggggagagtcatgaccatcccgtaccagcccatgccggccagctcgccagtcatctgcgcgggcggccaagatcgctgcagcgacgcggcgggctacccccacgccacccacgacctggagggaccacctctagacgcctactcgattcaaggacaacacaccatttctccgctggatctggccaagctgaaccaggtggcaagacaacagtctcactttgccatgatgcacggcgggacgggattcgccggaattgactccagctctccagaggtgaaaggctattgggcaagtttggatgcatctactcaaaccacccatgaactcaccattccaaataacttaatcggctgcataatcgggcgccaaggcgccaacatcaatgagatccgccagatgtccggggcccagatcaaaattgccaacccggtggaaggctcttctggaaggcaggtcactattactggctcggctgccagtattagcctggcccagtatctaatcaatgccaggctttcctctgagaaggggatggggtgcagctag

SEQ ID NO: 50 - DNA, 1071 bp, Homo sapiens
atggatgccggtgtgactgaaagtggactaaatgtgactctcaccattcggcttcttatgcacggaaaggaagtaggaagcatcattgggaagaaaggggagtcggttaagaggatccgcgaggagagtggcgcgcggatcaacatctcggaggggaattgtccggagagaatcatcactctgaccggccccaccaatgccatctttaaggctttcgctatgatcatcgacaagctggaggaagatatcaacagctccatgaccaacagtaccgcggccagcaggcccccggtcaccctgaggctggtggtgccggccacccagtgcggctccctgattgggaaaggcgggtgtaagatcaaagagatccgcgagagtacgggggcgcaggtccaggtggcgggggatatgctgcccaactccaccgagcgggccatcaccatcgctggcgtgccgcagtctgtcaccgagtgtgtcaagcagatttgcctggtcatgctggagacgctctcccagtctccgcaagggagagtcatgaccattccgtaccagcccatgccggccagctccccagtcatctgcgcgggcggccaagatcggtgcagcgacgctgcgggctacccccatgccacccatgacctggagggaccacctctagatgcctactcgattcaaggacaacacaccatttctccgctcgatctggccaagctgaaccaggtggcaagacaacagtctcactttgccatgatgcacggcgggaccggattcgccggaattgactccagctctccagaggtgaaaggctattgggcaagtttggatgcatctactcaaaccacccatgaactcaccattccaaataacttaattggctgcataatcgggcgccaaggcgccaacattaatgagatccgccagatgtccggggcccagatcaaaattgccaacccagtggaaggctcctctggtaggcaggttactatcactggctctgctgccagtattagtctggcccagtatctaatcaatgccaggctttcctctgagaagggcatggggtgcagctag

SEQ ID NO: 51 - DNA, 2313 bp, Mus musculus
atgctgcccagcttggcactgctcctgctggccgcctggacggttcgggctctggaggtacccactgatggcaacgccgggctgctggcagaaccccagatcgccatgttctgtggtaaactcaacatgcacatgaatgtgcagaatggaaagtgggagtcagacccgtcagggaccaaaacctgcattggcaccaaggagggcatcttgcagtactgccaagaggtctaccctgaactgcagatcacaaacgtggtggaagccaaccagccagtgaccatccagaactggtgcaagcggggccgcaagcagtgcaagacacacacccacatcgtgattccttaccgttgcctagttggtgagtttgtgagcgacgcccttctcgtgcccgacaagtgcaagttcctacaccaggagcggatggatgtttgtgagacccatcttcactggcacaccgtcgccaaagagacatgcagcgagaagagcactaacttgcacgactatggcatgctgctgccctgcggcatcgacaagttccgaggggtagagtttgtatgctgcccgttggccgaggaaagcgacagcgtggattctgcggatgcagaggaggatgactctgatgtctggtggggtggagcggacacagactacgctgatggcggtgaagacaaagtagtagaagtcgccgaagaggaggaagtggctgatgttgaggaagaggaagctgatgatgatgaggatgtggaggatggggacgaggtggaggaggaggccgaggagccctacgaagaggccaccgagagaacaaccagcactgccaccaccaccacaaccaccactgagtccgtggaggaggtggtccgagaggtgtgctctgaacaagccgagaccgggccatgccgcgcaatgatctcccgctggtactttgatgtcactgaagggaagtgtgtcccattcttttacggcggatgtggcggcaacaggaacaactttgacacggaagagtactgcatggcggtgtgtggcagcgtgtcaacccaaagtttactcaagactaccagtgaacctcttccccaagatcctgataaacttcccacgacagcagccagcacccccgacgccgtcgacaagtacctggagacacccggggacgagaacgagcatgcccatttccagaaagccaaagagaggctggaagccaagcaccgagagagaatgtcccaggtcatgagagaatgggaagaggcagagcgtcaagccaagaacttgcccaaagctgacaagaaggccgttatccagcatttccaggagaaagtggaatctctggaacaggaagcagccaatgagagacagcagcttgtagagacacacatggccagagttgaagccatgctcaatgaccgccgccgcctggccctcgagaattacatcactgcactgcaggcggtgcccccaaggcctcatcatgtgttcaacatgctgaagaagtacgtccgtgcggagcagaaagacagacagcacaccctaaagcattttgaacatgtgcgcatggtggaccccaagaaagctgctcagatccggtcccaggttatgacacacctccgtgtgatctacgagcgcatgaaccagtctctgtccctgctctacaatgtccctgcggtggctgaggagattcaagatgaagtcgatgagctgcttcagaaggagcagaactactccgacgatgtcttggccaacatgatcagtgagcccagaatcagctacggaaacgacgctctcatgccttcgctgacggaaaccaagaccaccgtggagctccttcccgtgaatggggaattcagcctggatgacctccagccgtggcacccttttggggtggactctgtgccagccaataccgaaaatgaagtcgagcctgttgacgcccgccccgctgctgaccgaggactgaccactcgaccaggttctgggctgacaaacatcaagacggaagagatctcggaagtgaagatggatgcagaattcggacatgattcaggatttgaagtccgccatcaaaaactggtgttctttgctgaagatgtgggttcgaacaaaggcgccatcatcggactcatggtgggcggcgttgtcatagcaaccgtgattgtcatcaccctggtgatgttgaagaagaaacagtacacatccatccatcatggcgtggtggaggtcgacgccgccgtgaccccagaggagcgccatctctccaagatgcagcagaacggatatgagaatccaacttacaagttctttgagcaaatgcagaactaa

SEQ ID NO: 52 - DNA, 2313 bp, Homo sapiens
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 53 - DNA, 2975 bp, Mus musculus
atggcggaacccgggaccgggtcgggcgaccccgcctttggtccgggcgcctcggagagtgggacgcggcggctcagcgacctgcgggtgatcgacctgagggcggagctgaaaaagcggaacctggatacgggaggcaacaagagcgtgctgatggagcggctgaggaaggcatttaaggaggaaggacaagaacctgaggaggttggaatcagctggggagctgtgagcaagagggctgtaaagagaaacaccaaaggatccaagatggaggaggagggcagcgaggacaacggcctggaggaagattccagatatgggcaggatggggttgtgattcttcagagttcacaagacagggacaccatggatacaggtgtgccagatgggatggaggctgaggacctcagtgtgccctgcctggggaaggccgacaccgtcaaccagattcttcatgcttttgatgacagtaaagagtacgtggccgcgcagctgggacagcttccagctcagctcctgaagcatgctgtggatgaggaggtcttcaagaacactctggaagcttccgtgtcagaccttaaagtaactctggctgatgaagaagcccccatggaaccagaaaatgagaaaatactcgacattttgggggaaacttgtaaatctgagccagtaaaagaagaaggttcggagctggagcagccctttgcccaggccacgagtagcgtggggccagacaggaagctggcggaggaagaggacctatttgagagctgcggccacccggaagaggaagaggaagaggaggaagaagatcaggaagaggagcaggaggaggagggagatttagctttggccagcagcagcaagtctgagtctccaagcactcggtgtcagtggagcgaggcagatgccccgttagcagtagtgaaaagggagctggcagatgcgccgggtggaggtggaggtactcggcacaggcggaagcgcaagcgaaggcggaagcatcaggcgcaggcagaagctctgggcacaggtggaggtgccgggatgaactgcgagcctgtagggctagaggagccagttgaacagagtagcacggctgcccagctcccggaggccaccagccaggagctggtgcgagcgcccacggcagccctgagccctgagccccaagatagcaaagaagacgtgaagaagtttgcttttgacgcttgtaatgacgtccctgcgcctcctaaagagtcctcagccagtgagggcgctgatcagaaaatgagctctgttaaggaagaacaagatataaagccagtcattaaagatgaaaaaggccgtgccagctgtagctcaggaaggaacctgtgggtcagtgggctgtcctccagcacccgtgctgctgacctcaagagcctcttcagcaagcatgggaaggtcatcggagccaaggtggtcaccaatgcccgcagtccaggggctcggtgctatggatttgtgacaatgtccacgtcagatgaggccaccaaatgcattagccacctgcacagaactgaactacatgggaggatgatctctgtggagaaggccaagaatgaaccctctgagaagaagtcgtcagacaggagagcatgtgaccaaaaggagaaggtgccagggccggacaggcctcaccctgtgaagatcaagacggagaaaactgtgatcaagaaggaagaaaaactggaaagaaaagaggagaaagggccagaagacattaaaaaggagaaggatcaagatgaactcacaccaggagctgctggtcattctcgagtcaccaaatcaggaagcagaggcatggagcgtacagtcgtgatggacaagtccaagggcgagcccgtcatcagtgtgaaggccacaagcaggtcaaaggacagaagctccaaaagtcaagaccgaaagtcagagggcagggagaagagagacatactgtcattcgacaagatcaaggagcagcgggagagggagcgccagaggcagagggagcgtgaaatccgggagacagagcgaagacgggagcgggagcagcgagagcgggagcagcgtctggatgccttccaggagcgacgggagaaggctcgcttgcagcgggaacggatgcagctccagtgccagcggcagcggttggagcgggagcggctggagcgggagaggctggagcgggagcgcatgagagtggagcgtgaaaggcgcaaggaacagcagcgcattatgcgtgagcgggaggagctgcggcggcagcaagaacagctgcgtgctgagcaggaacggcgggcactgcgcagaccctacgacctggatgctaggagagacgatggctactggccagaagggaagcgtgcagccttagaggacagataccgagactttccacggccagatcaccgcttccatgactttgatcaccgagatcgtggccattaccaggagcatgtcatagacaggcgggatgggtccagaaccagagtggaggagcgggatgggcagtactacccagatgaccagcacagccatggaaggctcctagagcaccatgcttgggattccggagacggctggcatggctacagctctgacaagaagttgaatgaaggccaagggctaccccctccccccagggtcagccgagagtgggcggagcacagctcacagttggaggagcagcaggttcctgtctggcacagtgctgtggacacaaacatgacgggccatgaacacatacggtggcgaggtgctgagcggggccttgcaggaggacctgggcatgggcatgtggcagcaggccggggtggcatggctgggcaaggcagctttgcacacggtggacattcccagggctatattgtgcccagtggtagactggaaggtggtggcatggccagccaggaccagggtggccgagttcctaacccccaccctcacccccacttcacccgccgctactg

SEQ ID NO: 54 - DNA, 2862 bp, Homo sapiens
atggcggagactctgcccgggtcgggcgactcgggccctggcacggcttctctcggcccgggcgttgcggagactgggacgaggcggctcagcgagctgcgggtgatcgatctgcgggcggagctgaagaagcggaacctggacacgggcggcaacaagagcgtcctgatggagcggctcaagaaggcggttaaagaagaggggcaagatcctgatgaaattggcatcgagttagaagccaccagcaagaagtcagccaagagatgtgttaaaggactgaagatggaggaggaaggcacagaagataatggcctggaagacgattccagagacgggcaggaggacatggaagcaagtctggagaacctgcagaatatgggcatgatggacatgagtgtgctagacgaaactgaagtggcgaatagcagtgctccagattttggggaggatggcacggacggccttctcgattccttttgtgatagtaaagaatacgtggctgcacagctgagacagctcccggctcagcccccagagcatgctgtggatggggaaggatttaagaacactttggaaacttcatcgttgaacttcaaagtaactccggacattgaagaatcccttttggagccagaaaatgagaaaatactcgacattttgggggaaacttgtaaatctgagccagtaaaagaagaaagttccgagctggagcagccatttgcacaggacacaagtagcgtggggccagacagaaagcttgcggaggaagaggacctatttgacagcgcccatccggaagagggtgatttagatttggccagcgagtcaacagcacacgctcagtcgagcaaggcagacagcctgttagcggtagtgaaaagggagcccgcggagcagccaggcgatggcgagaggacggactgtgagcctgtagggctagagccggcagttgagcagagtagtgcggcctccgagctcgcggaggcctctagcgaggagctcgcagaagcacccacggaagccccaagcccagaagccagagatagcaaagaagacgggaggaagtttgattttgacgcttgtaatgaagtccctccggctcctaaagagtcctcaaccagtgagggcgctgatcagaaaatgagctcttttaaggaagaaaaagatataaagccaatcattaaagatgaaaaaggtcgggtcggcagcggttctggtcggaacctgtgggtcagcgggctgtcctccacaacacgcgctacggatctcaagaaccttttcagcaagtatgggaaggttgtcggggccaaagtggtaacgaacgcccgcagcccgggggctcgatgctatggattcgtcaccatgtcgacatctgacgaggcgaccaagtgcatcagccatctccacagaactgagctgcatggacgaatgatctccgtagagaaggccaaaaatgagcctgctgggaaaaagctttccgacagaaaagagtgcgaagtgaagaaggaaaaattatcgagtgtcgacagacatcattctgtggagatcaaaattgaaaaaactgtaattaagaaggaagagaagattgagaagaaggaggaaaaaaagcctgaagacattaagaaggaagaaaaagaccaggatgagctgaaacccggacctacaaatcggtctagagtcaccaaatcaggaagcagaggaatggagcggacggtcgtgatggataaatcgaaaggagagcccgtcattagcgtgaaaaccacaagcaggtccaaagagagaagctccaagagtcaggatcgcaagtcagaaagcaaagaaaagagagacatcttgtcgtttgataaaatcaaagaacaaagggagagagagcgccagaggcagcgggaacgggagatccgcgaaacggagaggcggcgggagcgcgagcagcgggagcgggagcaacgcctcgaggccttccatgagcggaaggagaaggcccggctacagcgggaacgcctgcagctcgagtgccagcgccagcggctggagcgggagcgcatggagcgggagcggctggagcgcgagcgcatgcgcgtggagcgtgagcgcaggaaggagcaggagcgcatccaccgcgagcgcgaggagctgcggcgccagcaggagcagctgcgttacgagcaggagcggcggcccgggcggaggccctacgacctggaccgacgagatgatgcctattggccagaaggaaagcgtgtggcaatggaggaccgatatcgtgcagactttccccggccagaccaccgctttcacgacttcgatcatcgagaccggggccagtaccaggaccacgccatcgacaggcgggagggttcgaggccaatgatgggagaccaccgggatgggcagcactatggagatgaccgccatggccacggaggacccccagagcgccacggccgggactcccgtgatggctgggggggctacggctccgacaagaggctgagtgaaggccgggggctgccccctccccccaggggtggccgtgactggggagagcacaaccagcggctagaggagcaccaggcacgcgcctggcagggtgccatggacgcaggcgcggctagccgggagcacgccaggtggcaaggtggcgagaggggcctgtctgggccctcggggccggggcacatggcaagccgcggtggagtggcggggcgaggcggctttgcacaaggtggacattcccagggccacgtggtgccaggtggcggactggaaggtggcggagtggccagccaggaccggggcagcagagtccctcacccacaccctcatccccccccgtacccccacttcacccgccgctactaa

SEQ ID NO: 55 - DNA, 2346 bp, Mus musculus
atggctactcaagctgacctgatggagttggacatggccatggagccggacagaaaagctgctgtcagccactggcagcagcagtcttacttggattctggaatccattctggtgccaccaccacagctccttccctgagtggcaagggcaaccctgaggaagaagatgttgacacctcccaagtcctttatgaatgggagcaaggcttttcccagtccttcacgcaagagcaagtagctgatattgacgggcagtatgcaatgactagggctcagagggtccgagctgccatgttccctgagacgctagatgagggcatgcagatcccatccacgcagtttgacgctgctcatcccactaatgtccagcgcttggctgaaccatcacagatgttgaaacatgcagttgtcaatttgattaactatcaggatgacgcggaacttgccacacgtgcaattcctgagctgacaaaactgctaaacgatgaggaccaggtggtagttaataaagctgctgttatggtccatcagctttccaaaaaggaagcttccagacatgccatcatgcgctcccctcagatggtgtctgccattgtacgcaccatgcagaatacaaatgatgtagagacagctcgttgtactgctgggacccttcacaacctttctcaccaccgcgagggcttgctggccatctttaagtctggtggcatcccagcgctggtgaaaatgcttgggtcaccagtggattctgtactgttctacgccatcacgacactgcataatctcctgctccatcaggaaggagctaaaatggcagtgcgcctagctggtggactgcagaaaatggttgctttgctcaacaaaacaaacgtgaaattcttggctattacaacagactgccttcagatcttagcttatggcaatcaagagagcaagctcatcattctggccagtggtggaccccaagccttagtaaacataatgaggacctacacttatgagaagcttctgtggaccacaagcagagtgctgaaagtgctgtctgtctgctctagcaacaagccggccattgtagaagctggtgggatgcaggcactggggcttcatctgacagacccaagtcagcgacttgttcaaaactgtctttggactctcagaaacctttcagatgcagcgactaagcaggaagggatggaaggcctccttgggactctagtgcagcttctgggttccgatgatataaatgtggtcacctgtgcagctggaattctctctaacctcacttgcaataattacaaaaacaagatgatggtgtgccaagtgggtggcatagaggctcttgtacgcaccgtccttcgtgctggtgacagggaagacatcactgagcctgccatctgtgctcttcgtcatctgaccagccggcatcaggaagccgagatggcccagaatgccgttcgccttcattatggactgcctgttgtggttaaactcctgcacccaccatcccactggcctctgataaaggcaactgttggattgattcgaaaccttgccctttgcccagcaaatcatgcgcctttgcgggaacagggtgctattccacgactagttcagctgcttgtacgagcacatcaggacacccaacggcgcacctccatgggtggaacgcagcagcagtttgtggagggcgtgcgcatggaggaaatagtcgaagggtgtactggagctctccacatccttgctcgggacgttcacaaccggattgtaatccgaggactcaataccattccattgtttgtgcagttgctttattctcccattgaaaatatccaaagagtagctgcaggggtcctctgtgaacttgctcaggacaaggaggctgcagaggccattgaagctgagggagccacagctcccctgacagagttactccactccaggaatgaaggcgtggcaacatacgcagctgctgtcctattccgaatgtctgaggacaagccacaggattacaagaagcggctttcagtcgagctgaccagttccctcttcaggacagagccaatggcttggaatgagactgcagatcttggactggacattggtgcccagggagaagcccttggatatcgccaggatgatcccagctaccgttcttttcactctggtggatacggccaggatgccttggggatggaccctatgatggagcatgagatgggtggccaccaccctggtgctgactatccagttgatgggctgcctgatctgggacacgcccaggacctcatggatgggctgcccccaggtgatagcaatcagctggcctggtttgatactgacctgtaa

SEQ ID NO: 56 - DNA, 2346 bp, Homo sapiens
atggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaa

SEQ ID NO: 57 - DNA, 4431 bp, Mus musculus
atgaagtctttgttaaatgccttcaccaagaaggaagtgcccttcagagaggccccagcttattccaaccgcaggcggcggccccccaacacattggctgcccccagagttcttctgcgttctaatagtgacaacaacctcaatgctggtgcacctgaatgggctgtctgctctgcagccacctcccaccgaagcctctcaccccaactgctgcagcagacacccagcaaacctgatggagccaccaagagccttggaagctatacccctgggccccgcagccgttccccttcactcaacaggctgggtggtactgctgaggatggcaagaggacacagccacattggcatgtggggtcgcccttcactcctggtgccaacaaggactccctctcgacctttgagtacccagggcccaggaggaagctatatagtgcggtgcctgggagactcttcgtcgccgtcaagccataccaaccccaagtcgacggcgagatcccccttcaccgaggcgacagggtcaaagttctgagcatcggagagggcggcttctgggaaggcagtgcccgtggccacatcgggtggttcccagctgagtgtgtggaagaggtgcagtgtaaaccccgggacagccaggcagaaacccgcgcggaccgcagcaagaagctcttccggcattacacggtgggctcctatgacagctttgatgctgccagtgactgcattattgaggacaagacggtggtcctgcaaaagaaggacaacgagggctttggatttgtgctccgaggggcaaaagccgatacccccattgaggaattcacacccacgccagcatttccagccctgcagtacctggagtccgtggatgaaggtggggtggcatggcaagccggactaaggaccggggacttcttgattgaggttaacaatgaaaatgtcgtcaaggtgggccacaggcaggtggtgaacatgatccgccagggagggaatcacctcatccttaaggtcgtcacggtgaccaggaatctagaccctgatgatacagccagaaagaaagctcccccacctccaaagcgggctccgaccacggccctcaccctgcgttccaagtccatgacagcggagttggaggaactcggcctctcactagtggacaaagcctcagtccggaagaagaaggataaaccggaagagatagtcccagcctccaagccctccaggactgcagagaacgtggccatcgaatccagggtggcgaccatcaagcagcggcccacaagccggtgcttcccagctgcctctgatgtgaattccgtgtacgagcgccaagggattgctgtaatgacgcccacggtccctgggagcccgaaaggcccatttctgggcctccctcgaggtacgatgcgaaggcagaaatcgatagacagcagaatctttctatcagggataacagaggaagagcggcagtttctggctcccccaatgctgaagttcacccgaagcttgtccatgccagacacttctgaggacatcccccctccgccacagtctgtgcccccctctccccctcctccttcccccaccacatacaactgtcccaggtccccgactccaagagtctatgggacaattaagcccgcgttcaatcagaaccccgtcgtcgccaaggtgcccccagccaccaggtctgacactgtggccaccatgatgcgggaaaaggggatgttctacaggagagagctggacagattttccctggactcagaagacgtctacagccgcagccccgccccacaggccgccttccgcaccaagcggggacagatgcctgagaacccgtactcagaggtgggaaagatagccagcaaggccgtctatgtccctgccaagccagccaggcggaagggcgtgctggtaaagcagtccaacgtggaggatagccccgagaagacgtgctccatacccatcccaaccatcatcgtcaaggaaccctccaccagcagcagcggcaagagcagccaggggagcagcatggagattgacccccaggccaccgagcccggccagctgcggccagatgacagcctcaccgtcagcagcccctttgctgcggccatcgctggggctgtgcgtgaccgggagaagcgtctggaagccaggaggaattccccagccttcctctccaccgacctgggagatgaggacgtgggtctggggccgcctgctccccggatgcaggcctccaagttcccagaggagggtgggtttggtgacgaggatgagacggaacaaccgctgttgcctacccccggggcagcacccagggagctggagaatcacttcctaggtggtggtgaggctggtgctcagggggaggctgggggacccctgagttccacatccaaagccaaggggcctgagagtggcccagcagcccccctcaagagcagcagcccagccggccctgagaattacgtgcacccactcacagggcggctgcttgaccccagctccccactggccctggcactctcagccagggaccgagccatgcaggagtcccagcaggggcacaaaggagaggcccccaaggctgaccttaacaagcctctctacatcgataccaaaatgcggcccagcgtggagtccggctttcctccagtcaccagacagaacaccaggggtcccctgcgacggcaagagacagagaacaagtacgagacagacctgggcaaggaccggagggcggacgacaagaagaacatgctgatcaacatcgtggacactgcccagcagaagtcagctggcctactgatggtgcacacagtggacgtccccatggccgggccacccctggaggaagaggaggacagagaggatggggatacaaagccagaccactcaccctccacagtgccagaaggcgttcccaaaaccgaaggtgctttacagatctccgctgccccggagcccgccgtcgcgcccggcaggaccatcgtggcggcgggctccgtggaagaggcggtgattctgccattccgcatcccccctccccctctggcatccgtggacttggatgaggactttcttttcacagaaccgttgcctccccccctggaattcgccaatagttttgatatccccgatgaccgggcagcttcagttcccgctctggctgacctggtcaagcagaagaaaaacgacacctctcagccccctacgttgaactccagccaaccagccaactccacagacagtaagaagccagccggtatctcgaactgtctgccctcctcgttcctgccaccccccgaaagtttcgatgcagtcaccgactcggggattgaggaggtggacagccggagtagcagcgaccaccacctggagactaccagcaccatctccacggtgtccagcatctccacgctgtcctcagagggcggcgagagcatggacacctgcacagtctatgcagacgggcaagcctttgtggttgacaagcccccagtacctccaaagccaaaaatgaagcccatcgttcacaagagcaacgcactttaccaagacacgctcccagaagaggacacggatggctttgtgatccccccacctgcacccccgcccccgccgggcagcgcccaggccggtgtggcgaaggtcatccagccaaggacctccaagttgtggggtgacgttccagaggtcaaaagcccaattctctcaggcccaaaggcaaatgtcattagtgagctaaactccattctgcagcagatgaacagggggaaatcggtcaagcccggggaagggctggagctgccggtgggagccaagtcggccaacctcgctccaagaagcccggaggtcatgagcaccgtctcaggtacacggagcacgacggtcaccttcactgtccgccctggcacctcccagcccatcaccctgcagagccggccccccgactatgaaagcagaacctcaggacctagacgcgccccaagccctgtggtttcaccaacggaattgagcaaagagatcctgcccacccctccccctccgtccgccactgcagcctctccctcccccacactctcagatgtctttagccttccgagccagtcccctgcaggggacctctttggcttgaacccagcaggacggagcaggtcaccatctccttcaatattgcaacagccaatctcaaataagccttttacaactaagcctgtccacctgtggacgaaaccagatgtggcagactggctggaaagtctgaacttgggtgaacacaaggagacgttcatggacaatgagattgacggcagccacctgccaaaccttcagaaggaagacttgatagatcttggggtgactcgagttgggcataggatgaacatagaaagggctttgaaacagctgctggacagataa

SEQ ID NO: 58 - DNA, 4413 bp, Homo sapiens
atgaagtctttgttaaatgccttcaccaagaaggaagtgcccttccgagaggccccggcgtactccaaccgccggcggcggccccccaacacgctggccgccccccgggtcctgctgcgctccaacagtgacaacaacctcaatgccagcgctcccgactgggccgtctgctccacggccacctcgcaccgcagcctgtcaccccagctgctgcagcagatgcccagcaagcccgagggggccgcgaagaccattgggagctacgtgcccgggccccgcagccggtccccatcgctcaacaggctgggcggcgcaggcgaggacggcaagaggccgcagcctctctggcatgtcgggtcgccttttgctcttggtgccaacaaggactcactctcggccttcgagtacccggggcccaagcggaagctctacagtgccgtgcccgggaggctcttcgtcgctgtcaagccataccaaccccaagtggacggcgagatcccccttcaccgcggtgacagggtcaaagttctgagcatcggtgaagggggcttctgggaaggcagcgcccgcggccacatcggatggtttccggcggagtgcgtggaggaggtccagtgtaagcccagggacagccaggcagaaacccgcgctgaccgcagcaagaagcttttcaggcactacaccgtgggctcctatgacagcttcgacacttccagtgactgcattattgaggagaagacggtggtcctgcagaaaaaagacaatgagggctttggattcgtgcttcgaggggccaaagctgacacacccattgaagaattcacaccaacaccggctttcccagccctacagtacctggagtccgtggatgaaggtggggtggcgtggcaagccggactaaggaccggggacttcttgattgaggttaacaatgagaatgttgtcaaagtcggccacaggcaggtggtgaacatgatccggcagggagggaatcacctggtccttaaggtggtcacggtgaccaggaatctggaccccgacgacaccgccaggaagaaagctcccccgcctccaaagcgggcaccgaccacagccctcaccctgcgctccaagtccatgacctcggagctggaggagctcgtggataaagcctcggtccggaagaagaaggataaacccgaggagatagtcccggcctccaagccctcccgcgctgctgagaacatggctgtggaaccgagggtggcgaccatcaagcagcggcccagcagccggtgcttcccggcgggctcagacatgaactctgtgtacgaacgccaaggaatcgccgtgatgacgcccactgttcctgggagcccaaaagccccgtttctgggcatccctcgaggtacgatgcgaaggcagaaatcaatagacagcagaatctttctatcaggaataacagaggaagagcggcagtttctggctcctccaatgctgaagttcaccagaagcctgtccatgccggacacctctgaggacatcccccctccaccgcagtctgtgcccccgtccccaccaccaccttccccaaccacttacaactgccccaagtccccaactccaagagtctacgggacgattaagcctgcgttcaatcagaattctgccgccaaggtgtcccccgccaccaggtccgacaccgtggccaccatgatgagggagaaggggatgtacttcaggagagagctggaccgctactccttggactctgaagacctctacagtcggaatgccggcccgcaagccaacttccgcaacaagagaggccagatgccagaaaacccatactcagaggtggggaagatcgccagcaaagccgtctacgtccccgccaagcccgccaggcggaaggggatgctggtgaagcagtccaacgtggaggacagccccgagaagacgtgctccatccctatcccgaccatcatcgtgaaggagccgtccaccagcagcagcggcaagagcagccagggcagcagcatggagatcgacccccaggccccggagccaccgagccagctgcggcctgacgaaagcctgaccgtcagcagcccctttgccgccgccatcgccggagccgtccgcgaccgtgagaagcggctggaagccaggaggaactccccggccttcctctccacagacctgggggatgaggatgtgggcctggggccacccgcccccaggacgcggccctccatgttccccgaggagggggattttgctgacgaggacagcgctgagcagctgtcatcccccatgccgagtgccacgcccagggagcccgaaaaccatttcgtgggtggcgccgaggccagtgctccgggtgaggctgggaggccgctgaattccacgtccaaagcccaggggcccgagagcagcccagcagtgccctccgcgagcagcggcacagccggccccgggaattatgtccacccactcacagggcggctgcttgatcccagctccccgctggccctggcactctccgcaagggaccgagccatgaaggagtctcaacagggacccaaaggggaggcccccaaggccgacctcaacaaacctctttacattgataccaaaatgcggcccagcctggatgccggcttccctacggtcaccaggcagaacacccggggacccctgaggcggcaggagacggagaacaagtacgagaccgacctgggccgagaccggaaaggcgatgacaagaagaacatgctgatcgacatcatggacacgtcccagcagaagtcggctggcctgctgatggtgcacaccgtggacgccactaagctggacaacgccctgcaggaagaggacgagaaggcagaggtggagatgaagccagacagctcgccgtccgaggtgccagaaggtgtttccgaaaccgaaggtgctttacagatctccgctgcccccgagcccaccaccgtgcccggcagaaccatcgtcgcggtgggctccatggaagaggcggtgattttgccattccgcatccctcctccccctctggcatccgtggacttggatgaggattttatttttacagagccattgcctcctcccctggaatttgcaaatagttttgatatccccgatgaccgggcagcttctgtcccggctctctcagacttagtgaagcagaagaaaagcgacacccctcagtccccttcgttgaactccagccaaccaaccaactctgcagacagcaagaagccagccagtctttcaaactgtctgcctgcctcattcctgccaccccctgaaagctttgacgccgtcgccgactctgggatcgaggaggtggacagccggagtagcagcgaccaccacctcgagacgaccagcactatctccaccgtgtctagcatctccaccctgtcttccgaaggtggagagaatgtggacacctgcacagtctatgcagatgggcaagcatttatggttgacaaacccccagtacctcctaagccaaaaatgaagcccatcattcacaaaagcaatgcactttatcaagacgcgctcgtggaagaagatgtagatagctttgttatccccccgcccgctcccccgcccccgccgggcagtgcccagcctgggatggccaaggttctccagccaaggacctccaagttgtggggcgacgtcacagagatcaaaagcccgattctctcaggcccaaaggcaaacgttattagtgaattgaactctatcctacagcaaatgaaccgagagaaattggcaaagccgggggaaggactggattcaccaatgggagccaagtccgccagcctcgctccaagaagcccggagatcatgagcaccatctcaggtacacggagcacgacggtcaccttcactgttcgccccggcacctcccagcccatcaccctgcagagccggccccccgactatgaaagcaggacctcaggaacaagacgtgccccaagccctgtggtctcgccaacagagatgaacaaagagaccctgcccgcccccctgtctgctgccaccgcctctccttctcccgctctctcagatgtctttagccttccaagccagcccccttctggggatctatttggcttgaacccagcgggacgcagtaggtcgccatccccctcgatactgcaacagccaatctcaaataagccttttacaactaaacctgtccacctgtggactaaaccagatgtggccgattggctggaaagtctaaacttgggtgaacataaagaggccttcatggacaatgagatcgatggcagtcacttaccaaacctgcagaaggaggacctcatcgatcttggggtaactcgagtcgggcacagaatgaacatagaaagggctttgaaacagctgctggacagataa

SEQ ID NO: 59 - DNA, 1212 bp, Mus musculus
atgacagccatcatcaaagagatcgttagcagaaacaaaaggagatatcaagaggatggattcgacttagacttgacctatatttatccaaatattattgctatgggatttcctgcagaaagacttgaaggtgtatacaggaacaatattgatgatgtagtaaggtttttggattcaaagcataaaaaccattacaagatatacaatctatgtgctgagagacattatgacaccgccaaatttaactgcagagttgcacagtatccttttgaagaccataacccaccacagctagaacttatcaaacccttctgtgaagatcttgaccaatggctaagtgaagatgacaatcatgttgcagcaattcactgtaaagctggaaagggacggactggtgtaatgatttgtgcatatttattgcatcggggcaaatttttaaaggcacaagaggccctagatttttatggggaagtaaggaccagagacaaaaagggagtcacaattcccagtcagaggcgctatgtatattattatagctacctgctaaaaaatcacctggattacagacccgtggcactgctgtttcacaagatgatgtttgaaactattccaatgttcagtggcggaacttgcaatcctcagtttgtggtctgccagctaaaggtgaagatatattcctccaattcaggacccacgcggcgggaggacaagttcatgtactttgagttccctcagccattgcctgtgtgtggtgatatcaaagtagagttcttccacaaacagaacaagatgctcaaaaaggacaaaatgtttcacttttgggtaaatacgttcttcataccaggaccagaggaaacctcagaaaaagtggaaaatggaagtctttgtgatcaggaaatcgatagcatttgcagtatagagcgtgcagataatgacaaggagtatcttgtactcaccctaacaaaaaacgatcttgacaaagcaaacaaagacaaggccaaccgatacttctctccaaattttaaggtgaaactatactttacaaaaacagtagaggagccatcaaatccagaggctagcagttcaacttctgtgactccagatgttagtgacaatgaacctgatcattatagatattctgacaccactgactctgatccagagaatgaaccttttgatgaagatcagcattcacaaattacaaaagtctga

SEQ ID NO: 60 - DNA, 1212 bp, Homo sapiens
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 61 - DNA, 351 bp, Mus musculus
atgtcggctaccgctgccaccgtcccgcctgccgccccggccggcgagggtggcccccctgcacctcctccaaaccttactagtaacaggagactgcagcagacccaggcccaggtggatgaggtggtggacatcatgagggtgaatgtggacaaggtcctggagcgggaccagaagttgtcggagctggatgaccgtgcagatgccctccaggcaggggcctcccagtttgaaacaagtgcagccaagctcaagcgcaaatactggtggaaaaacctcaagatgatgatcatcttgggagtgatctgcgccatcatcctcatcatcatcatcgtttacttcagcacttaa

SEQ ID NO: 62 - DNA, 351 bp, Homo sapiens
atgtctgctaccgctgccacggccccccctgctgccccggctggggagggtggtccccctgcaccccctccaaacctcaccagtaacaggagactgcagcagacccaggcccaggtggatgaggtggtggacatcatgagggtgaacgtggacaaggtcctggagcgagaccagaagctgtcggagctggacgaccgtgcagatgcactccaggcgggggcctcccagtttgaaacaagcgcagccaagctcaagcgcaaatactggtggaaaaacctcaagatgatgatcatcttgggagtgatttgcgccatcatcctcatcatcatcatagtttacttcagcacttaa

SEQ ID NO: 63 - DNA, 981 bp, Mus musculus
atgtctaatggttatgaagaccacatggcggaagactgcagggatgacattgggagaacgaatttaattgtcaactacctccctcagaacatgacccaagaggaactacgaagtctgttcagcagcattggcgaggttgaatctgcaaagcttattcgggataaagtagcaggacacagcttgggctacggttttgtgaactatgtgactgcaaaagatgcagagagagcaatcagcacactgaacggcttgagactccagtccaaaaccattaaggtgtcatatgctcgcccaagctcagaggtcatcaaagatgccaacttatacatcagtgggctcccaaggaccatgacacagaaggatgtggaagacatgttttctcggtttgggcgaatcatcaactccagggtccttgtggatcagaccacaggtttgtccagaggggttgcctttatccggtttgacaaacggtcagaagcagaagaggcaattaccagtttcaatggtcataaacccccaggttcctccgagcccatcacagtgaagtttgcagccaatcccaaccagaacaaaaacatggctctcctctcgcagctgtaccactcgcctgctaggcggtttggaggccctgtacaccaccaggcacagagattcaggttctcccctatgggtgtagatcacatgagtgggatttctggtgtcaatgtccccggcaatgcttcctcgggctggtgcatcttcatctacaaccttgggcaagacgccgatgaggggatcctctggcagatgtttggcccctttggtgcagttaccaatgtgaaagtgattcgtgatttcaacaccaacaagtgcaaagggtttggttttgtgaccatgacaaactatgaagaagctgcaatggccatagcaagtctgaacggctaccgcctgggggacaaaattttacaggtttccttcaaaaccaacaagtcccacaaataa

SEQ ID NO: 64 - DNA, 981 bp, Homo sapiens
atgtctaatggttatgaagaccacatggccgaagactgcaggggtgacatcgggagaacgaatttgatcgtcaactacctccctcagaacatgacccaggatgagttacgaagcctgttcagcagcattggtgaagttgaatctgcaaaacttattcgggataaagtagcaggacacagcttgggctatggctttgtgaactacgtgaccgcgaaggatgcagagagagcgatcaacacgctgaacggcttgaggctccagtcaaaaaccattaaggtgtcgtatgctcgcccgagctcagaggtgatcaaagacgccaacttgtacatcagcgggctcccgcggaccatgacccagaaggacgtagaagacatgttctctcggtttgggcggatcatcaactcgcgggtcctcgtggatcagactacaggtttgtccagaggggttgcgtttatccggtttgacaaacggtcggaggcagaagaggcaattaccagtttcaatggtcataaacccccaggttcctctgagcccatcacagtgaagtttgcagccaaccccaaccagaacaaaaacgtggcactcctctcgcagctgtaccactcgccagcgcgacggttcggaggccccgttcaccaccaggcgcagagattcaggttctcccccatgggcgtcgatcacatgagcgggctctctggcgtcaacgtgccaggaaacgcctcctccggctggtgcattttcatctacaacctggggcaggatgccgacgaggggatcctctggcagatgtttgggccgtttggtgccgtcaccaatgtgaaagtgatccgcgacttcaacaccaacaagtgcaaagggtttggctttgtgaccatgacaaactatgaagaagccgcgatggccatagccagcctgaacggctaccgcctgggggacaaaatcttacaggtttccttcaaaaccaacaagtcccacaaataa

SEQ ID NO: 65 - DNA, 4839 bp, Mus musculus
atgatcgcggagcctgctcacttttacctctttggattaatatgtctctgttcaggctcccgtcttcgtcaggaagattttccaccccgcattgttgaacacccttcagacctgatcgtctccaaaggagaacctgccactctgaactgtaaagccgaaggccgccccacccccactatagaatggtacaaaggtggggaaagagtggagaccgacaaagatgacccgcgctcacaccggatgctgctgccaagcgggtctttatttttcttacgcatagtccatggacggaagagtaggcccgacgaaggggtctacatctgtgtggctaggaattacctcggagaggctgtgagccacaacgcatcgctggaggtagctatactacgggatgacttcagacaaaacccttcagatgtcatggtggcagtaggcgaacctgcagtcatggaatgccagcctccacggggccatccagagcccaccatctcctggaaaaaggatggctcacccctggatgataaagatgaaagaatcactatacgaggaggaaaactcatgatcacttatacgcgtaaaagcgatgctggcaagtatgtttgcgttggcaccaacatggtgggggaacgggagagtgaagtcgcagagctaaccgttttagagagaccatcgtttgtgaagagacccagtaatttggcagtgactgtggatgacagtgcagaatttaaatgtgaggcccgaggagaccctgtgcctacagttcgatggaggaaagatgacggagagctgcccaaatccagatatgaaatccgagatgatcatactttgaaaattcggaaggtgactgctggcgacatgggatcatacacttgtgtggcagaaaatatggtcgggaaagctgaagcttccgctactttgacagttcaagagccgccacatttcgttgtaaaacctcgggaccaggtggttgccttgggaagaactgtgacgtttcagtgcgaagcaactggaaatcctcaaccagctatcttttggaggagagaggggagtcagaatctacttttctcctaccagcctccacagtcctctagccgattttcagtttcgcagacaggagacctcaccattacgaacgtccagcggtctgatgtcggctactacatctgtcagactttaaatgttgctggaagtatcatcacaaaggcctatttggaagttactgatgtgattgcagaccggcctcccccagtcattcggcaaggtcctgtgaatcagactgtagcagtggatggcactttaatcctcagctgtgtggccacaggcagtccagcacccacaattctgtggaggaaggatggagtccttgtttcaacccaagattctcggatcaaacagctggagagtggcgtgctgcagatccgctacgctaagctgggtgacacgggtcggtacacctgcaccgcatcgacccccagcggtgaggccacgtggagcgcttacatcgaagtccaggaatttggggttccagttcaacccccaagacccaccgaccccaacttaatccccagcgccccctcaaagcccgaagttacagatgtcagcaaaaacacggtcactttatcgtggcagccaaacttgaactcaggagcaaccccaacatcttatattatagaagccttcagccatgcatctggtagcagctggcagacggcggcagaaaatgtgaaaacagaaacatttgccatcaaaggactcaaacctaacgccatttaccttttcctggtgagggcagcaaatgcctatggaattagtgatccaagccaaatatcagatccagtgaaaacgcaagatgtcccaccaaccagccaaggggtggaccacaagcaggtccaaagagaactggggaatgttgtcttacatctgcacaacccgaccatcctttcttcctcctcagtcgaagtgcactggacggtggaccaacaatctcaatatattcaaggctataaaattctctaccgcccatctggagccagccatggtgaatcggagtggttagtttttgaagtgaggacgccaacgaaaaacagcgtggtgatccccgatctcagaaaaggcgtcaactacgagattaaggctcgcccattttttaatgagtttcaaggagcagacagtgaaatcaaatttgccaaaaccttagaagaagcaccaagtgcccctccccgaagtgtaactgtatccaagaatgatggaaatgggactgcaatcctggtcacttggcaaccgcctcctgaagacacacaaaatggaatggtccaagaatataaggtttggtgtctcggtaatgaaacgaagtatcacatcaacaagacagtcgatggctcgaccttctcggtggtcatcccctctctggttcctgggatccgatacagtgtggaggtggcagcgagcactggggcgggtcccggggtgaagagcgaacctcagttcatccagttagattctcacggaaaccccgtgtctcccgaggaccaagtcagccttgctcagcagatctccgacgtggtgaggcagccggcgttcattgcaggcattggggcagcctgttggattatcctcatggtcttcagcatctggctttacagacaccgcaagaagagaaacggactcaccagcacgtatgcgggtatccggaaagtcccgtcctttaccttcacgccaacagtaacttatcaaagaggaggcgaagccgtcagcagtggagggaggccgggactcctcaacattagtgaacctgccacgcagccatggctggcagacacgtggcctaacacaggcaacaaccacaatgactgctccatcaactgctgcacggccggcaatggcaacagcgacagcaacctgacaacctacagtcgcccagctgattgtatagccaattataacaaccaactggataacaaacaaacaaatctgatgctccctgagtcaactgtttatggtgatgtggaccttagtaacaaaatcaatgagatgaaaaccttcaatagcccaaatctgaaggacgggcgttttgtcaatccatcagggcagcccactccctatgccaccactcaactcatccaggcgaacctcagcaacaatatgaataatggcgccggggactccagcgagaagcactggaagcctccgggacagcagaaaccggaagtggcaccaattcagtataacatcatggagcaaaacaagctgaacaaagattatcgggcaaatgacacgatccctccaaccatcccatacaaccagtcctatgaccagaacacaggagggtcttataacagctcagatcggggcagtagtacatccgggagtcaaggccacaagaaaggggcacggacaccaaaggcacccaagcaaggtggcatgaactgggcagacttgctgccccctcctccggcgcatcctcccccacatagcaacagtgaagaatacaacatgtccgtagatgaaagctatgatcaagaaatgccatgtccagtgccacctgcgccgatgtatttgcaacaggatgaattacaggaagaggaagatgaaagaggccccactccccctgtccggggggcagcttcttctccagctgctgtgtcctacagccaccaatccacggccactctcaccccttctccccaggaagagctccagcccatgttacaggattgtccggaggacctcgggcacatgccccacccaccagacaggaggcggcagcccgtgagtcctcctccaccgccacggccaatttccccacctcatacttacggctacatttcaggaccccttgtctcagatatggataccgatgccccagaagaggaggaggacgaagctgacatggaagttgccaaaatgcagacccgaaggctgttgttacgtgggctggagcagaccccagcatccagtgttggggaccttgagagctccgtcactgggtccatgatcaatggctggggctcagcctcggaagaggacaacatttccagtgggcgctccagtgtcagttcatccgatggctcctttttcactgacgctgattttgcccaggcagttgctgcagctgcggagtatgcgggcctgaaagtggctcgccgccaaatgcaagatgctgctggccgccgccacttccatgcctctcagtgcccaaggcccacgagtcctgtgtccacagacagcaacatgagtgctgttgtgatccagaaagccagacccgccaagaagcagaaacaccagccaggacatctgcgcagggaagcctacgcagatgatcttccaccccctccagtgccaccacctgctataaaatcgcccactgtccagtccaaggcacagctggaggtacggcctgtcatggtgccaaaactcgcgtctatagaagcaaggacagatagatcgtcagacagaaaaggaggcagttacaaggggagagaagctctggatggaagacaagtcactgacctgcgaacaaatccaagtgaccccagagaggcacaggaacagccaaatgacgggaaaggccgaggaacaaggcagcccaagcgagaccttccaccagcaaagacgcacctcggccaagaggatattcttccgtactgtagacctactttcccaacgtcaaacaaccctagagaccccagttcctccagctctatgtcatcaagaggatcagggagccgacaaagagagcaagcaaatgtgggccgaaggaatatggcagaaatgcaagtacttgggggatttgaaagaggagatgagaataatgaagaattagaggaaactgaaagctga

SEQ ID NO: 66 - DNA, 4956 bp, Homo sapiens
atgaaatggaaacatgttccttttttggtcatgatatcactcctcagcttatccccaaatcacctgtttctggcccagcttattccagaccctgaagatgtagagagggggaacgaccacgggacgccaatccccacctctgataacgatgacaattcgctgggctatacaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtgaagagacccagtaacttggcagtaactgtggatgacagtgcagaatttaaatgtgaggcccgaggtgaccctgtacctacagtacgatggaggaaagatgatggagagctgcccaaatccagatatgaaatccgagatgatcataccttgaaaattaggaaggtgacagctggtgacatgggttcatacacttgtgttgcagaaaatatggtgggcaaagctgaagcatctgctactctgactgttcaagaacctccacattttgttgtgaaaccccgtgaccaggttgttgctttgggacggactgtaacttttcagtgtgaagcaaccggaaatcctcaaccagctattttctggaggagagaagggagtcagaatctacttttctcatatcaaccaccacagtcatccagccgattttcagtctcccagactggcgacctcacaattactaatgtccagcgatctgatgttggttattacatctgccagactttaaatgttgctggaagcatcatcacaaaggcatatttggaagttacagatgtgattgcagatcggcctcccccagttattcgacaaggtcctgtgaatcagactgtagccgtggatggcactttcgtcctcagctgtgtggccacaggcagtccagtgcccaccattctgtggagaaaggatggagtcctcgtttcaacccaagactctcgaatcaaacagttggagaatggagtactgcagatccgatatgctaagctgggtgatactggtcggtacacctgcattgcatcaacccccagtggtgaagcaacatggagtgcttacattgaagttcaagaatttggagttccagttcagcctccaagacctactgacccaaatttaatccctagtgccccatcaaaacctgaagtgacagatgtcagcagaaatacagtcacattatcgtggcaaccaaatttgaattcaggagcaactccaacatcttatattatagaagccttcagccatgcatctggtagcagctggcagaccgtagcagagaatgtgaaaacagaaacatctgccattaaaggactcaaacctaatgcaatttaccttttccttgtgagggcagctaatgcatatggaattagtgatccaagccaaatatcagatccagtgaaaacacaagatgtcctaccaacaagtcagggggtggaccacaagcaggtccagagagagctgggaaatgctgttctgcacctccacaaccccaccgtcctttcttcctcttccatcgaagtgcactggacagtagatcaacagtctcagtatatacaaggatataaaattctctatcggccatctggagccaaccacggagaatcagactggttagtttttgaagtgaggacgccagccaaaaacagtgtggtaatccctgatctcagaaagggagtcaactatgaaattaaggctcgccctttttttaatgaatttcaaggagcagatagtgaaatcaagtttgccaaaaccctggaagaagcacccagtgccccaccccaaggtgtaactgtatccaagaatgatggaaacggaactgcaattctagttagttggcagccacctccagaagacactcaaaatggaatggtccaagagtataaggtttggtgtctgggcaatgaaactcgataccacatcaacaaaacagtggatggttccaccttttccgtggtcattccctttcttgttcctggaatccgatacagtgtggaagtggcagccagcactggggctgggtctggggtaaagagtgagcctcagttcatccagctggatgcccatggaaaccctgtgtcacctgaggaccaagtcagcctcgctcagcagatttcagatgtggtgaagcagccggccttcatagcaggtattggagcagcctgttggatcatcctcatggtcttcagcatctggctttatcgacaccgcaagaagagaaacggacttactagtacctacgcgggtatcagaaaagtcccgtcttttaccttcacaccaacagtaacttaccagagaggaggcgaagctgtcagcagtggagggaggcctggacttctcaacatcagtgaacctgccgcgcagccatggctggcagacacgtggcctaatactggcaacaaccacaatgactgctccatcagctgctgcacggcaggcaatggaaacagcgacagcaacctcactacctacagtcgcccagctgattgtatagcaaattataacaaccaactggataacaaacaaacaaatctgatgctccctgagtcaactgtttatggtgatgtggaccttagtaacaaaatcaatgagatgaaaaccttcaatagcccaaatctgaaggatgggcgttttgtcaatccatcagggcagcctactccttacgccaccactcagctcatccagtcaaacctcagcaacaacatgaacaatggcagcggggactctggcgagaagcactggaaaccactgggacagcagaaacaagaagtggcaccagttcagtacaacatcgtggagcaaaacaagctgaacaaagattatcgagcaaatgacacagttcctccaactatcccatacaaccaatcatacgaccagaacacaggaggatcctacaacagctcagaccggggcagtagtacatctgggagtcaggggcacaagaaaggggcaagaacacccaaggtaccaaaacagggtggcatgaactgggcagacctgcttcctcctcccccagcacatcctcctccacacagcaatagcgaagagtacaacatttctgtagatgaaagctatgaccaagaaatgccatgtcccgtgccaccagcaaggatgtatttgcaacaagatgaattagaagaggaggaagatgaacgaggccccactccccctgttcggggagcagcttcttctccagctgccgtgtcctatagccatcagtccactgccactctgactccctccccacaggaagaactccagcccatgttacaggattgtccagaggagactggccacatgcagcaccagcccgacaggagacggcagcctgtgagtcctcctccaccaccacggccgatctcccctccacatacctatggctacatttcaggacccctggtctcagatatggatacggatgcgccagaagaggaagaagacgaagccgacatggaggtagccaagatgcaaaccagaaggcttttgttacgtgggcttgagcagacacctgcctccagtgttggggacctggagagctctgtcacggggtccatgatcaacggctggggctcagcctcagaggaggacaacatttccagcggacgctccagtgttagttcttcggacggctcctttttcactgatgctgactttgcccaggcagtcgcagcagcggcagagtatgctggtctgaaagtagcacgacggcaaatgcaggatgctgctggccgtcgacattttcatgcgtctcagtgccctaggcccacaagtcccgtgtctacagacagcaacatgagtgccgccgtaatgcagaaaaccagaccagccaagaaactgaaacaccagccaggacatctgcgcagagaaacctacacagatgatcttccaccacctcctgtgccgccacctgctataaagtcacctactgcccaatccaagacacagctggaagtacgacctgtagtggtgccaaaactcccttctatggatgcaagaacagacagatcatcagacagaaaaggaagcagttacaaggggagagaagtgttggatggaagacaggttgttgacatgcgaacaaatccaggtgatcccagagaagcacaggaacagcaaaatgacgggaaaggacgtggaaacaaggcagcaaaacgagaccttccaccagcaaagactcatctcatccaagaggatattctaccttattgtagacctacttttccaacatcaaataatcccagagatcccagttcctcaagctcaatgtcatcaagaggatcaggaagcagacaaagagaacaagcaaatgtaggtcgaagaaatattgcagaaatgcaggtacttggaggatatgaaagaggagaagataataatgaagaattagaggaaactgaaagctga

(19) United States
(12) Patent Application Publication
(10) Pub. No.: US 2020/0172904 A1
(43) Pub. Date: Jun. 4, 2020

(54) METHODS OF MODULATING PROTEIN EXPRESSION FROM THE MENA-RIBONUCLEOPROTEIN COMPLEX IN CELLS

(71) Applicant: Massachusetts Institute of Technology, Cambridge, MA (US)

(72) Inventors: Frank B. Gertler, Boston, MA (US); Marina Vidaki, Belmont, MA (US)

(21) Appl. No.: 16/629,942

(22) PCT Filed: Jul. 9, 2018
(86) PCT No.: PCT/US2018/041344

§ 371 Date: Jan. 9, 2020

Related U.S. Application Data
(60) Provisional application No. 62/530,637, filed on Jul. 10, 2017.

(51) Int. Cl. C12N 15/113; A61K 31/7088

(52) U.S. Cl. CPC C12N 15/113; C12N 2310/11; C12N 2310/14; A61K 31/7088

(57) ABSTRACT

The present disclosure provides a method of modulating protein expression from a Mena-ribonucleoprotein (RNP) complex, the method comprising administering to a subject an agent that: (a) inhibits protein expression by inhibiting Mena translation, Mena transcription, or the association of Mena with the Mena-RNP complex, or (b) promotes protein expression by: (i) inhibiting the expression of at least one of HnmpK, PCBP1, or both, or (ii) dissociating at least one of HnmpK, PCBP1, or both, from the Mena-RNP complex in the cell. The present disclosure also provides a method of ameliorating, treating, or preventing at least one symptom of a disease, disorder, or syndrome associated with the overexpression or accumulation of DYRK1A and/or amyloid precursor protein (APP) in cells, as well as a method of ameliorating, treating, or preventing at least one symptom of a disease, disorder, or syndrome associated with the underexpression DYRK1A in cells.

### CROSS-REFERENCE TO RELATED APPLICATIONS

The present disclosure claims priority to U.S. Provisional Patent Application No. 62/530,637, filed 10 Jul. 2017, which is incorporated herein by reference in its entirety for all purposes.

### STATEMENT OF GOVERNMENT FUNDING

The present disclosure was made with government support under U01-CA184897 awarded by the National Institutes of Health. The government has certain rights in the invention.

### INCORPORATION BY REFERENCE PARAGRAPH

In compliance with 37 C.F.R. § 1.52(e)(5), the sequence information contained in electronic file name: 1515028_109WO2_Sequence_Listing_ST25.txt; size 221 KB; created on: 5 Jul. 2018; using Patent-In 3.5, and Checker 4.4.0 is hereby incorporated herein by reference in its entirety.

### BACKGROUND

### 1. Field of the Invention

The present disclosure relates to methods for treating neurodevelopmental defects, cognitive disorders, and other pathologies (e.g., cancer) arising from increased protein expression of DYRK1A and/or other proteins that are regulated by the Mena-ribonucleoprotein (RNP) complex.

### 2. Background of the Art

During embryonic development, the exquisitely regulated process of axon guidance establishes the circuitry necessary for a properly functioning nervous system (NS) in the adult. Aberrant axonal navigation results in defective connectivity and multiple neurodevelopmental disorders including, among others, epilepsy, intellectual disabilities, autism and schizophrenia (McCandless, 2012; Sahin and Sur, 2015; Wegiel et al., 2010). The growth cone, a specialized structure at the distal tip of growing axons must continuously sample the microenvironment for guidance cues and integrate this information rapidly into appropriate motility responses, frequently without sufficient time for transcriptional responses. Indeed, axons severed from their cell bodies can navigate correctly in vivo, and respond to guidance cues in vitro (Batista and Hengst, 2016; Campbell et al., 2001; Verma et al., 2005). Local mRNA translation is a key mechanism in such autonomous responses, and protein synthesis inhibitors block the ability of growth cones severed from their somata to respond to several guidance cues (Batista and Hengst, 2016; Jung et al., 2012). However, most of the present understanding of regulated local protein synthesis is based on the characterization of individual mRNAs found in axons (Deglincerti and Jaffrey, 2012; Kim and Jung, 2015), with few details of the underlying molecular mechanism. Even in synapses, where local translation has been studied intensely, only a handful of proteins have been identified as key regulators of local mRNA translation (Bassell and Warren, 2008; Brown et al., 2001; Darnell et al., 2011; De Rubeis and Bagni, 2010; Deglincerti and Jaffrey, 2012; Fritzsche et al., 2013; Hutten et al., 2014; Kindler et al., 2012).

Mena (also known as ENAH), a member of the Ena/VASP family of proteins, is highly expressed in the developing and adult NS, and is a known regulator of actin dynamics, integrin-mediated signaling, adhesion and cell motility (Bear and Gertler, 2009; Drees and Gertler, 2008; Gupton and Gertler, 2010). Mena and its paralogs, VASP and EVL, are required for normal NS development during neurulation (Lanier et al., 1999; Menzies et al., 2004), neuritogenesis (Kwiatkowski et al., 2007), migration (Goh et al., 2002; Kwiatkowski et al., 2007), axon guidance responses to both attractive and repulsive signals (Bashaw et al., 2000; Dent et al., 2011; Dent and Gertler, 2003; Kwiatkowski et al., 2007; Mcconnell et al., 2016), terminal axon branching (Lebrand et al., 2004), dendritic morphology and synapse formation (Li et al., 2005; Lin et al., 2007). Of the three Ena/VASP proteins, Mena is the most abundant in the NS, and Mena-null animals exhibit clear defects in NS development, while VASP/EVL double mutants exhibit no obvious NS phenotypes in animals with a wild type Mena allele (Kwiatkowski et al., 2007). Further, while Ena/VASP family proteins share a highly-conserved domain structure, Mena contains additional domains and alternatively-included sequences not found in VASP or EVL (Gertler and Condeelis, 2011).

As such, a need exists to be able to modulate protein expression in cells, such as neuronal cells. For example, dysregulation of protein expression can result in overexpressed, accumulation, or underexpression in a cell relative to the level of a normal individual, thereby causing a disease, disorder or syndrome. Thus, a need exist to more specifically modulate protein expression.

The present disclosure identifies a ribonucleoprotein (RNP) complex containing Mena, known translation regulators, and specific cytosolic mRNAs, including dyrk1a. Dyrk1a, a dual specificity kinase with multiple roles in neuronal development, has been implicated in the pathology and etiology of Down Syndrome, autism, intellectual disabilities, along with Alzheimer's and Parkinson's disease (Coutadeur et al., 2015; Di Vona et al., 2015; Krumm et al., 2014; O'Roak et al., 2012; Qian et al., 2013; Tejedor and Hammerle, 2011; van Bon et al., 2015). The present disclosure identifies ways in which to modulate protein expression, such as Dyrk1a expression, from the Mena-RNP complex.

### SUMMARY OF THE INVENTION

It was surprising and unexpectedly discovered that Mena is present within a novel ribonucleoprotein (RNP) complex containing the established translational repressors HnrnpK and PCGP1, along with cytosolic mRNAs in developing neurons as well as in non-neuronal cell types. It was also surprising and unexpectedly discovered that certain mRNAs (e.g., dyrk1a) are locally translated in a Mena-dependent manner.

In an aspect, the present disclosure provides a method of modulating protein expression from a Mena-ribonucleoprotein (RNP) complex, the method comprising administering to a subject an agent that: (a) inhibits protein expression by inhibiting Mena translation, Mena transcription, or the association of Mena with the Mena-RNP complex, or (b) promotes protein expression by: (i) inhibiting the expression of at least one of HnmpK, PCBP1, or both, or (ii) dissociating at least one of HnmpK, PCBP1, or both, from, or preventing the association of at least one of HnmpK, PCBP1, or both with, the Mena-RNP complex in the cell.

In some embodiments, the agent that inhibits protein expression is selected from an RNAi agent, an antibody or an antigen binding fragment thereof, peptide or a small molecule directed to Mena.

In certain embodiments, the agent that inhibits protein expression inhibits DYRK1A expression in the cell.

In other embodiments, the agent that promotes protein expression is an agent the results in increased levels of brain derived neurotrophic factor (BDNF) in the cell.

In particular embodiments, the agent that promotes protein expression is an RNAi agent directed to at least one of HnmpK, PCBP1, or both.

In further embodiments, the cell is a neuron.

In yet other embodiments, the administering step results in the modulation of the translation of an mRNA selected from Table 3.

In another aspect, the present disclosure provides a method of ameliorating, treating, or preventing at least one symptom of a disease, disorder, or syndrome associated with the overexpression or accumulation of DYRK1A and/or amyloid precursor protein (APP), the method comprising providing a subject in need thereof, and administering an effective amount of an agent that inhibits Mena translation, Mena transcription, or the association of Mena with the Mena-RNP complex, wherein the method is effective for ameliorating, treating, or preventing at least one symptom of a disease, disorder, or syndrome associated with the overexpression or accumulation of DYRK1A and/or amyloid precursor protein (APP).

In some embodiments, the cell is a neuron.

In other embodiments, the disease, disorder, or syndrome is selected from the group consisting of a cognitive disorder, Down Syndrome, Alzheimer's disease, Parkinson's disease, or cancer.

In certain embodiments, the cancer is hematological malignancy or brain cancer.

In certain other embodiments, the cancer is breast cancer, pancreatic cancer, lung cancer, or colon cancer.

In further embodiments, the agent that inhibits protein expression is selected from an RNAi agent, an antibody or an antigen binding fragment thereof, or a small molecule directed to Mena.

In a further aspect, the present disclosure provides a method of ameliorating, treating, or preventing at least one symptom of a disease, disorder, or syndrome associated with underexpression of DYRK1A, the method comprising providing a subject in need thereof, and administering an effective amount of an agent that promotes protein expression by (i) inhibiting the expression of at least one of HnmpK, PCBP1, or both, or (ii) dissociating at least one of HnmpK, PCBP1, or both, from, or preventing the association of at least one of HnmpK, PCBP1, or both with, the Mena-RNP complex in the cell, wherein the method is effective for ameliorating, treating, or preventing at least one symptom of a disease, disorder, or syndrome associated with underexpression of DYRK1A.

In some embodiments, the agent that promotes protein expression is an agent the results in increased levels of brain derived neurotrophic factor (BDNF) in the neuron.

In other embodiments, the agent that promotes protein expression is an RNAi agent directed to at least one of HnmpK, PCBP1, or both.

In further embodiments, the cell is a neuron.

In any aspect or embodiments described herein, the subject is selected from the group consisting of a cell, a mammal, and a human.

In yet a further aspect, the present disclosure provides a method of diagnosing a subject as having a Mena-RNP complex associated disease, disorder, or syndrome the method comprising: obtaining or providing a sample from the subject; detecting the expression level of the protein in the sample from the subject; comparing the expression level in the sample to a control having normal expression levels of the protein; and diagnosing the subject as having a disease, disorder, or syndrome associated with the dysregulation of the expression of the protein when the sample has increased or decreased expression relative to the control, wherein the protein is at least one protein selected from Table 3.

In some embodiment, the method further comprises administering to the subject an agent that: (a) inhibits protein expression by inhibiting Mena translation, Mena transcription, or the association of Mena with the Mena-RNP complex, or (b) promotes protein expression by: (i) inhibiting the expression of at least one of HnmpK, PCBP1, or both, or (ii) dissociating at least one of HnmpK, PCBP1, or both, from (or preventing the association of at least one of HnmpK, PCBP1, or both with) the Mena-RNP complex in a cell.

In certain embodiments, the agent that inhibits protein expression is selected from an antisense agent, an RNAi agent, an antibody or an antigen binding fragment thereof, peptide or a small molecule directed to Mena. For example, the agent that inhibits protein expression may be a peptide or pepido-mimetic that mimics and/or competes for Mena EVH1-ligand binding.

In particular embodiments, the agent that inhibits Mena translation, Mena transcription, or the association of Mena with the Mena-RNP complex inhibits expression of the protein in the cell.

In other embodiments, the agent that promotes protein expression is an agent the results in increased levels of brain derived neurotrophic factor (BDNF) in the cell.

In further embodiments, the agent that promotes protein expression is an RNAi agent directed to at least one of HnmpK, PCBP1, or both.

In any aspect or embodiment of the present disclosure, the cell is a neuron.

In any aspect or embodiment of the present disclosure, the administering step results in the modulation of the translation of an mRNA selected from Table 3.

In another embodiment, detecting the expression level of the protein comprises detecting the protein, which may be accomplished via at least one of immunohistochemistry, enzyme-linked immunosorbent assay, western blot, or a combination thereof.

In yet further embodiments, detecting the expression level of the protein comprises detecting mRNA of the protein, which may be accomplished via at least one of fluorescent in situ hybridization, northern blot, reverse-transcription polymerase chain reaction (RT-PCR), RT real time PCT, microarray, or a combination thereof.

In a particular embodiment, the subject is selected from the group consisting of a cell, a mammal, and a human.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure may be utilized in numerous combinations, all of which are expressly contemplated by the present disclosure. These additional advantages objects and embodiments are expressly included within the scope of the present disclosure. The publications and other materials used herein to illuminate the background of the invention, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention.

FIG. 1 is Table 2. Proteins interacting with Mena in developing mouse brains.

FIGS. 2A, 2B, 2C, 2D, 2E, and 2F illustrate that Mena interacts with RBPs and cytosolic mRNAs. FIG. 2A. Mass Spectrometry analysis of Mena-IP assays from E15.5 whole brain lysates, revealed a subset of RBPs interacting with Mena. FIG. 2B. Mena interacts with RBPs in N2A cells. FIG. 2C. Mena interacts with SafB2 in MEFs; efforts to detect specific association of Mena with HnrnpK and with PCBP1 in MEFs yielded inconsistent results. FIG. 2D. Schematic representation of the Oligo(dT) pulldown assays. FIG. 2E. Oligo(dT) pulldown assays from MEFS revealed that Mena is associated with cytosolic mRNAs in a non-neuronal cell type. FIG. 2F. Distribution of peaks from the Mena HITS-CLIP on the transcriptome. Although the vast majority or reads mapped to the gene region, a small number of reads mapped to UTRs within the mRNAs

FIGS. 3A, 3B, 3C, 3D, 3E, and 3F demonstrate that Mena interacts with RNA binding proteins and cytosolic mRNAs in the brain. FIG. 3A. CoIP validation of RBPs that associate with Mena in developing mouse brains. Panels show western blots, probed with antibodies to the indicated proteins, of Mena and IgG2a isotype control IPs and of 5% input lysate. FIG. 3B. Mena is associated with cytosolic mRNAs. Proteins enriched in Oligo(dT) pulldowns, analyzed by western blot probed with antibodies to Mena and to positive control RBPs, FMR1 and MBNL1, as indicated. FIG. 3C. Schematic representation of the modified HITS-CLIP protocol. E15.5 mouse brain tissues were triturated and UV-crosslinked to preserve RNP-complexes, and then homogenized in mild lysis buffer to generate lysates for Mena-IP. Co-IPed RNA was subsequently isolated and processed for sequencing. FIG. 3D. Gene-Set-Enrichment-Analysis (REACTOME) of the mRNAs identified through Mena HITS-CLIP revealed enrichment of categories relevant to previously known Mena functions (i.e. axon guidance). Only the mRNAs that had more than 10 reads and 3-fold enrichment between the Mena and control IP samples were used for the analysis. FIG. 3E. qPCR validation of several mRNAs that specifically associated with Mena. Irrelevant antibodies and Mena null brains were used as experimental controls. The graph represents relative mRNA enrichment of Mena-Associated mRNAs between the wt and mve samples±StDEV (Student's T test p*<0.05). FIG. 3F. Peaks in the 3′UTR of mena and dyrk1a indicate a regulatory role of the interaction between Mena and the mRNAs.

FIGS. 4A, 4B, 4C, 4D, 4E, and 4F illustrate that dyrk1a mRNA co-localizes with Mena in neuronal growth cones and axons. FIG. 4A. Combined IF for Mena (a) and FISH of dyrk1a mRNA (b) on E15.5+2DIV cultured mouse cortical neurons revealed significant overlap of the two signals in axons and growth cones (d). In contrast, Mena (a′) and a control FISH probe (species specific for human dyrk1a mRNA) (b′), fail to co-localize (d′). Ai, Aii. Higher magnification of filopodia showing co-localization between Mena and dyrk1a mRNA (white arrows). Phalloidin staining for F-actin (c,c′) was used to visualize morphology. FIG. 4B. Line scans along (i) or across (ii) stained filopodia (indicative dashed white lines depicted in Ai and Aii). Fluorescence intensities from both signals (protein and mRNA) nicely coincide within growth cone filopodia. FIG. 4C. Pearson's coefficient correlation for the protein and mRNA signals over the entire growth cone. Co-localization between Mena and dyrk1a mRNA is significantly higher than co-localization between Mena and a control mRNA probe (Student's T test p***<0.001). The graph represents mean Pearson's r±StDEV. Scale bar for Aa-d and Aa′-d′: 5 μm, Ai-ii: 1 μm. FIG. 4D. An FP4-mito construct expressed in neurons (a-d) co-recruits the dyrk1a mRNA to the mitochondrial surface, in contrast to the control AP4-mito (a′-d′). Mena IF (b and b′), dyrk1a FISH (c & c′), F-actin staining and a merge of Mena IF+dyrk1a FISH (d,d′) are shown. FIG. 4E. Magnification of boxed inserts i and ii from D showing dyrk1a mRNA distribution with respect to the mitochondrial surface of FP4- and AP4-transfected neurons (white arrows in i and ii respectively). FIG. 4F. Pearson's coefficient correlation for the mRNA and mitochondrial signal was assessed to verify the significant difference between AP4- and FP4-mito (Student's T test p**<0.01). The graph represents mean Pearson's r±StDEV. Scale bar for Da-d and Da′-d′: 20 μm, Ei-ii: 5 μm.

FIGS. 5A, 5B, 5C, 5D, 5E, 5F, and 5G demonstrated that Mena is necessary and sufficient to relocalize dyrk1a to the mitochondria, unlike VASP that does not associate with dyrk1a. FIG. 5A. IF for Mena and Dyrk1a protein did not show significant overlap of the two signals. Scalebar: 5 μm. FIG. 5B. Schematic representation of the mitochondrial sequestration assay. Mena relocalizes to the mitochondrial surface, and so do proteins and mRNAs that are associated with it in the cell. FIG. 5C. The total mRNA levels of dyrk1a are not affected by FP4- and AP4-mito construct expression. FIG. 5D. Relocalization of Mena to the mitochondria did not affect the distribution of Dyrk1a protein in mitochondria-sequestration assays. FIG. 5E. RT-PCR after VASP-CLIP assays on E15.5 mouse brains revealed no interaction between VASP and certain Mena-RNP-associated mRNAs. The graph represents Mean±StDEV (Student's T test p<0.001). FIG. 5F. Mena is necessary for the relocalization of dyrk1a to the mitochondria, unlike VASP and Evl. Scalebar: 10 μm. FIG. 5G. Pearson's coefficient correlation for the mRNA and mitochondrial signal (Student's T test p***<0.001). The graph represents mean Pearson's r±StDEV.

FIGS. 6A, 6B, 6C, 6D, and 6E demonstrate the RBPs mediate the interaction between Mena and dyrk1a 3′UTR. FIG. 6A. Volcano plot of enriched hexamers within the Mena-associated 3′UTR sequences. Hexamers with a density higher in the Mena-HITS-CLIP compared to the control, have enrichments >1 (positive log values), whereas hexamers with densities lower in the Mena_HITS-CLIP than in the control, have enrichments <1 (negative log values). Interestingly, some of the top hits correspond to RBPs found associated with Mena, including HnrnpK, PCBP1 and Safb2. FIG. 6B. Schematic representation of the RNP-pulldown assay with the 3′UTR of dyrk1a mRNA as bait. FIG. 6C. Western blot analysis of the pulldown fraction revealed that Mena, Safb2, HnrnpK and PCBP1 can bind the 3′UTR of dyrk1a mRNA, unlike HnrnpA2B1, which was used as a negative control RBP. An RNA probe generated by in vitro transcription of λ-phage was used as a negative control bait. FIG. 6D. siRNA-mediated ablation of HnrnpK in neurons reduces signal overlap between Mena IF and dyrk1a FISH (large white arrows in ii), as opposed to control siRNAs (large white arrows in i). Smaller arrows in ii point to mRNA signal that does not overlap with Mena. FIG. 6E. Pearson's coefficient correlation for the FISH and IF signal was assessed to verify the significant difference between neurons with control- and hnrnpK-siRNAs (Student's T test p**<0.01). The graph represents mean Pearson's r±StDEV.

FIGS. 7A, 7B, and 7C demonstrate that part of Mena and dyrk1a association is HnrnpK-dependent. FIG. 7A. In silico-predicted binding sites for PCBP1, Safb2 and HnrnpK on the 3′UTR of dyrk1a. The graph shows predicted kmer motifs (left Y axis), within the dyrk1a 3′UTR-specific sequences, that could be recognized by PCBP1, HnrnpK and Safb2 and the probability of them to do so (−log 10 p value) (rbpmap.technion.ac.il). FIG. 7B. Colocalization of Mena, HnrnpK and dyrk1a mRNA in neuronal growth cones (white arrows in inserts 1-8). Scalebar: 5 μm. FIG. 7C. HnrnpK ablation with siRNA pools in cultured neurons (absence of HnrnpK signal in cells with the siRNA in b′.). White arrows in inserts indicate the colocalization of Mena and HnrnpK on filopodia. Scalebar: 20 μm.

FIGS. 8A and 8B demonstrated that mena, dyrk1a and other Mena-associated mRNAs are locally translated upon BDNF stimulation. FIG. 8A. Quantification of Mena and Dyrk1a IF signal in growth cones±BDNF stimulation. The graph represents Mean±StDEV (Two-Way Anova p*<0.05). FIG. 8B. Western blot analysis of additional Mena-associated mRNAs on unstimulated and BDNF-stimulated neurons after axotomy. Values were normalized to GAPDH loading controls and to the unstimulated protein levels to generate fold changes. The levels of the respective proteins were increased upon stimulus (Two-Way Anova p*<0.05). The graph represents Mean±StDEV.

FIGS. 9A, 9B, 9C, 9D, 9E, and 9F demonstrated that BDNF stimulation can induce local translation of Mena and Dyrk1a in axons. FIG. 9A. Schematic representation of the assay for local translation. FIG. 9B. Western blot analysis of the top and bottom filter compartments, verifies the presence of neuronal somata on the top (expression of Tbr1), and the enrichment in the bottom part of axons (pan Tau), but not dendrites (Map2). FIG. 9C. Protein levels of Mena and Dyrk1a increase after BDNF stimulation in whole cell lysates. FIG. 9D. Quantification of Mena and Dyrk1a proteins in whole cells demonstrated elevated protein levels upon BDNF stimulation, but not when translation was blocked by anisomycin. The graph represents Mean±StDEV (Two-Way Anova p<0.05). FIG. 9E. BDNF stimulation of axons only elicits a greater increase in the protein levels of both Mena and Dyrk1a in axonal lysates. FIG. 9F. Quantification of the proteins in isolated axonal preparations reveals significant changes upon BDNF stimulation. All values were normalized to loading controls (Gapdh) and then to the unstimulated protein levels to generate fold changes. The graph represents Mean±StDEV (Two-Way Anova p*<0.05).

FIGS. 10A, 10B, 10C, 10D, and 10E demonstrated that BDNF stimulation reduces the association between Mena and the mRNA of dyrk1a. FIG. 10A. IF for Mena and FISH for dyrk1a before and after BDNF stimulation of cortical neurons in culture (b, c and b′, c′ respectively). Co-localization of the signal is reduced after the BDNF stimulation (white arrows in magnified panels d and d′). Scale bar: 5 μm. FIG. 10B. Stimulation of neurons with BDNF results in a significant increase of total dyrk1a mRNA levels, both in the growth cones and in the proximal axon part (Student's T test p**<0.01). The graph represents Mean±StDEV. FIG. 10C. Pearson's coefficient correlation for the FISH and IF signal was assessed before and after BDNF treatment, revealing significant decrease in co-localization of FISH and IF signal after stimulation (Student's T test p<0.01). The graph represents Mean±StDEV. FIG. 10D. Neurons expressing the FP4-mito construct were processed for Mena IF and dyrk1a FISH, before (a-f) and after BDNF stimulation (a′-f′). Scale bar a-d and a′-d′: 20 μm; e-f and e′-f′: 5 μm. FIG. 10E. Pearson's coefficient correlation for the FISH and IF signal revealed significantly decreased mRNA signal co-recruited on the mitochondrial surface after BDNF stimulation (Student's T test p**<0.01; p***<0.001). The graph represents Mean±StDEV.

FIGS. 11A, 11B, 11C, 11D, 11E, and 11F demonstrate that the Mena-RNP complex is partially disassembled upon BDNF stimulation. FIG. 11A. Western blot analysis of protein coIP after Mena-IP on unstimulated and BDNF-stimulated neurons in culture. Inputs and precipitated fractions are of different exposure times. FIG. 11B. Significantly reduced amounts of HnrnpK, PCBP1 and Safb2 coIP with Mena after 15 minutes of BDNF stimulation, compared to the respective amounts of proteins interacting with Mena in unstimulated cells. Each precipitated protein value was normalized to its respective input and to the amount of precipitated Mena. The graph represents Mean±StDEV (Student's T test p*<0.05; p***<0.001). FIG. 11C. Western blot of biotinylated mRNA pulldown assays, before and after BDNF stimulation of neurons in culture (E15.5+2DIV). The 3′UTR of dyrk1a was used as bait, and the 3′UTR of lhx6 was used as a specificity control. FIG. 11D. Quantification of the protein levels in the inputs used for the assay, and in the mRNA pulldown fractions, revealed that there is significantly less binding of Mena, as well as HnrnpK and Pcbp1 on the 3′UTR of dyrk1a after BDNF stimulation. Input protein levels were normalized to the unstimulated lysate levels and the pulldown proteins were normalized to the respective input values. The graphs represent Mean±StDEV (Student's T test p*<0.05; p**<0.01; p***<0.001). FIG. 11E. IF for Mena and FISH for dyrk1a after BDNF stimulation of neurons that are HnrnpK-depleted. Overlap between the two signals is indicated by large white arrows in b and b′, whereas FISH signal not overlapping with Mena is shown by small white arrows. Scale bar: 5 μm. FIG. 11F. Pearson's coefficient correlation for the FISH and IF signal revealed significantly decreased co-localization, both under steady-state conditions and after BDNF stimulation in the HnrnpK-depleted background (Student's T test p**<0.01). The graph represents Mean±StDEV.

FIGS. 12A, 12B, 12C, 12D, 12E, 12F, and 12G demonstrate that the absence of Mena does not affect localization of dyrk1a mRNA, but significantly reduces both steady-state and BDNF-elicited increases in Dyrk1a protein levels. FIG. 12A. Western blots of whole brain lysates of different Mena genotypes (wt: Mena+/+; VASP−/−; EVL−/−, het: Mena+/−; VASP−/−; EVL−/−, mve: Mena−/_; VASP−/−; EVL−/−) showing a decrease in Dyrk1a protein levels in the absence of Mena. FIG. 12B. Quantification of the data of FIG. 12A. Protein levels are normalized to the wt protein amount. The graph represents Mean±StDEV (Student's T test p<0.05). FIG. 12C. Western blot analysis of axotomy assays to study protein levels of Dyrk1a in mve vs. wt axons before and after BDNF stimulation. FIG. 12D. Dyrk la protein levels are significantly decreased in mve axons and are not changed by BDNF stimulation. Values were normalized to the wt protein levels using the GAPDH loading controls. The graph represents Mean±StDEV (Two-Way Anova p*<0.05). FIG. 12E. FISH for dyrk1a mRNA on cultured cortical neurons (E15.5+2DIV) from wt and mve brains. Scalebar: 5 μm. FIG. 12F. Quantification of the fluorescence intensity revealed significant differences in the mRNA levels, between the axons and growth cones of wt and mve neurons, with the mRNA levels in the mutant cells being substantially increased. The graph represents Mean±StDEV (Student's T test p***<0.001). FIG. 12G. Quantitative PCR analysis with mRNA from wt and mve neurons, revealed a significant increase in the mRNA of dyrk1a present in the mutant axons and growth cones. The graph represents Mean±StDEV (Student's T test p**<0.01).

FIG. 13 demonstrates that Dyrk1a mRNA levels increase after protein unmasking. FISH signal after pepsin treatment of the samples increases significantly, as proteins that mask the mRNA are removed (Student's T test p***<0.001). The graph represents Mean±StDEV.

### DETAILED DESCRIPTION OF THE INVENTION

Mena, a member of the Ena/VASP family of proteins, is highly expressed in the developing nervous system and is a known regulator of actin dynamics, adhesion and cell motility. Genetic and biochemical evidence implicate Mena in neuronal migration and axon guidance downstream of both attractive and repulsive axon signals, and other evidence implicates Mena in synaptic formation and plasticity. Mena-null mice exhibit axon guidance and connectivity defects. Although Mena function in actin dynamics and adhesion is involved in axon extension and guidance, the present disclosure has identified a novel aspect of Mena function in regulation of local protein synthesis, that is relevant to nervous system development and function, with potential relevance to neurodevelopmental disorders, including, inter alia, Down's syndrome and Autism spectrum disorders. Axon growth and guidance responses are known to require local protein synthesis, however, the mechanisms that regulate local translation in response to guidance cues are only poorly understood. By analyzing Mena immunoprecipitates (IP) by mass spectrometry and HITS-CLIP (High-throughput sequencing of RNA isolated by crosslinking immunoprecipitation), the inventors of the present disclosure surprisingly discovered that Mena is present within a novel ribonucleoprotein (RNP) complex containing the established translational repressors HnrnpK and PCBP1, along with cytosolic mRNAs in developing neurons, as well as in non-neuronal cell types. The present disclosure identifies multiple transcripts associated with Mena in the cytoplasm of neurons, many of which are particularly important for axon growth and guidance, as well as synapse formation and plasticity. The present disclosure further discovered that certain mRNAs, such as the Down Syndrome-related kinase dyrk1a, are locally translated in axons upon stimulation with growth factors, in a Mena-dependent manner. In Mena-deficient neurons, dyrk1a fails to be translated upon stimulation and instead the mRNA accumulates in the axon. Further, analysis of brain lysates from Mena deficient mice indicates that steady state levels of the Dyrk1a proteins are significantly reduced to ˜50% of that observed in wildtype animals. Given the extreme dosage sensitivity of Dyrk1a and its implication in numerous neurodevelopmental disorders, like Down Syndrome, microcephaly, tumor growth, pancreatic dysfunction, etc., the present findings that Dyrk1a protein levels are regulated in a Mena-dependent manner in axons indicates that dysregulation of the Mena-RNP complex may contribute to such disorders. Additional mRNAs associated with the Mena-RNP complex, including β-catenin and elav11 (HuR), shank2, app, pten, etc, are also implicated in multiple developmental processes and pathophysiological conditions, including autism, epilepsy, intellectual disabilities, as well as cancer.

As such, it was surprising and unexpectedly discovered that not only does the Mena-RNP complex exists, but also that the Mena-RNP complex could be utilized to modulate the expression of certain proteins, such as Dyrk1a and the other proteins found in Table 3 below. Therefore, the Mena-RNP complex represents a target for the development of novel therapeutic strategies to control synthesis of proteins that contribute to multiple disease pathologies. For example, targeting the Mena-RNP complex can reduce levels of Dyrk1a and APP proteins in patients with Down's syndrome and Alzheimer's diseases. While protein synthesis inhibitors are used/in development for therapies, such inhibitors (e.g. rapamycin) can impact global protein synthesis. Targeting the Mena-RNP complex, as described herein, would be far more selective, affecting translation of only those mRNAs that are associated with the complex.

The present disclosure now will be described more fully hereinafter, but not all embodiments of the disclosure are shown. While the disclosure has been described with reference to exemplary embodiments, it will be understood by those skilled in the art that various changes may be made and equivalents may be substituted for elements thereof without departing from the scope of the disclosure. In addition, many modifications may be made to adapt methods to the teachings of the disclosure without departing from the essential scope thereof.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the 10 United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

As used herein, the ten “antibody” encompasses whole antibodies and fragments of whole antibodies wherein the fragments specifically bind to Mena. Antibody fragments include, but are not limited to, F(ab′)₂ and Fab′ fragments and single chain antibodies. F(ab′)₂ is an antigen binding fragment of an antibody molecule with deleted crystallizable fragment (Fc) region and preserved binding region. Fab′ is ½ of the F(ab′)₂ molecule possessing only ½ of the binding region. The term antibody is further meant to encompass polyclonal antibodies and monoclonal antibodies. Antibodies may be produced by techniques well known to those skilled in the art. Polyclonal antibody, for example, may be produced by immunizing a mouse, rabbit, or rat with purified polypeptides encoded by Mena, Mena^{INV} and/or Mena^{11a}. Monoclonal antibody may then be produced by removing the spleen from the immunized mouse, and fusing the spleen cells with myeloma cells to form a hybridoma which, when grown in culture, will produce a monoclonal antibody. The antibody can be, e.g., any of an IgA, IgD, IgE, IgG, or IgM antibody. The IgA antibody can be, e.g., an IgA1 or an IgA2 antibody. The IgG antibody can be, e.g., an IgG1, IgG2, IgG2a, IgG2b, IgG3 or IgG4 antibody. A combination of any of these antibodies subtypes can also be used. One consideration in selecting the type of antibody to be used is the size of the antibody. For example, the size of IgG is smaller than that of IgM allowing for greater penetration of IgG into tissues. The antibody can be a human antibody or a non-human antibody such as a rabbit antibody, a goat antibody or a mouse antibody. Antibodies can be “humanized” using standard recombinant DNA technique.

In an aspect, the present disclosure provides a method of modulating protein expression from a Mena-ribonucleoprotein (RNP) complex, the method comprising administering to a subject an agent that: (a) inhibits protein expression by inhibiting Mena translation, Mena transcription, or the association of Mena with the Mena-RNP complex, or (b) promotes protein expression by: (i) inhibiting the expression of at least one of HnmpK, PCBP1, or both, or (ii) dissociating at least one of HnmpK, PCBP1, or both, from (or preventing the association of at least one of HnmpK, PCBP1, or both with) the Mena-RNP complex in the cell, such as a neuron. In certain embodiments, the agent that inhibits protein expression inhibits DYRK1A expression in the cell.

In certain embodiments, the agent promotes protein expression expression by (i) inhibiting the expression of SAFB2, (ii) dissociating SAFB2 from the Mena-RNP complex in the cell, or (iii) preventing the association of SAFB2 with the Mena-RNP complex in the cell.

In certain other embodiments, the agent inhibits proteion expression by inhibiting SAFB2 translation, SAFB2 transcription, or the association of SAFB2 with the Mena-RNP complex.

The agent that inhibits protein expression may be selected from an antisense agent/molecule/oligonucleotide, an RNAi molecule/agent (such as a short interfering RNA (siRNA) agent/molecule/oligonucleotide or an short hairpin RNA (shRNA) agent/molecule/nucleotide), an antibody or an antigen binding fragment thereof, peptide or a small molecule directed to Mena.

The antisense agent or RNAi agent directed to Mena specifically inhibits the expression of Mena. The antisense or RNAi agent directed to HnmpK or PCBP1 specifically inhibits the expression of HnmpK or PCBP1, respectively. The shRNA agent of the present disclosure can be introduced into the cell by transduction with a carrier and/or vector. The antisense molecule or RNAi molecule can be comprised of nucleic acid (e.g., DNA or RNA) or nucleic acid mimetics (e.g., phosphorothionate mimetics) as are known in the art. Methods for treating tissue with these compositions are also known in the art. The antisense molecule or RNAi molecule of the disclosure can be added directly to the tissue in a pharmaceutical composition that preferably comprises an excipient that enhances penetration of the antisense molecule or RNAi molecule into the cell. The antisense molecule or RNAi of the disclosure can be expressed from a vector that is transfected into the cell/tissue. Such vectors are known in the art.

In an embodiment, the siRNA agent of the disclosure comprises a double-stranded portion (duplex). In an embodiment, the siRNA agent is 20-25 nucleotides in length. In an embodiment, the siRNA comprises a 19-21 core RNA duplex with a one or 2 nucleotide 3′ overhang on, independently, either one or both strands. The siRNA can be 5′ phosphorylated or not and may be modified with any of the known modifications in the art to improve efficacy and/or resistance to nuclease degradation. In an embodiment, the siRNA agent of the disclosure can be administered such that it is transfected into one or more cells. In one embodiment, a siRNA agent of the disclosure comprises a double-stranded RNA, wherein one strand of the double-stranded RNA is 80, 85, 90, 95 or 100% complementary to a portion of an RNA transcript of a gene encoding mammalian (e.g. human) gene of interest, such as Mena, HnmpK, or PCBP1. In another embodiment, a siRNA agent of the disclosure comprises a double-stranded RNA, wherein one strand of the RNA comprises a portion having a sequence the same as a portion of 18-25 consecutive nucleotides of an RNA transcript of a gene encoding mammalian Mena. In yet another embodiment, a siRNA agent of the disclosure comprises a double-stranded RNA, wherein both strands of RNA are connected by a non-nucleotide linker. Alternately, a siRNA agent of the disclosure comprises a double-stranded RNA, wherein both strands of RNA are connected by a nucleotide linker, such as a loop or stern loop structure.

In one embodiment, a single strand component of a siRNA agent of the disclosure is from 14 to 50 nucleotides in length. In another embodiment, a single strand component of a siRNA agent of the disclosure is 14, 15, 16, 17, 18, 19, 20, 21, 22, 23, 24, 25, 26, 27, or 28 nucleotides in length. In yet another embodiment, a single strand component of a siRNA agent of the disclosure is 21 nucleotides in length. In yet another embodiment, a single strand component of a siRNA agent of the disclosure is 22 nucleotides in length. In yet another embodiment, a single strand component of a siRNA agent of the disclosure is 23 nucleotides in length. In one embodiment, a siRNA agent of the disclosure is from 28 to 56 nucleotides in length. In another embodiment, a siRNA agent of the disclosure is 40, 41, 42, 43, 44, 45, 46, 47, 48, 49, 50, 51, or 52 nucleotides in length. In yet another embodiment, a siRNA agent of the disclosure is 46 nucleotides in length.

In some embodiments, an siRNA agent of the disclosure comprises at least one 2′-sugar modification. In certain embodiments, an siRNA agent of the disclosure comprises at least one nucleic acid base modification. In another embodiment, an siRNA agent of the disclosure comprises at least one phosphate backbone modification.

In some embodiments, RNAi inhibition of Mena, HnmpK, and/or PCBP1 is effected by a short hairpin RNA (shRNA). The shRNA agent of the disclosure can be introduced into the cell by transduction with a carrier and/or vector. In further embodiments, the carrier is a lipofection reagent. In another embodiment, the carrier is a nanoparticle reagent. In an embodiment, the vector is a lentiviral vector. In a further embodiment, the vector comprises a promoter. In yet another embodiment, the promoter is a U6 or H1 promoter. In further embodiments, the shRNA agent of the disclosure is encoded by the vector is a first nucleotide sequence ranging from 19-29 nucleotides complementary to the target gene, or mRNA (e.g., encoding Mena, HnmpK, and/or PCBP1). In yet other embodiments, the shRNA agent is encoded by the vector also comprises a short spacer of 4-15 nucleotides (a loop, which does not hybridize) and a 19-29 nucleotide sequence that is a reverse complement of the first nucleotide sequence. In particular embodiments, the siRNA agent that results from the intracellular processing of the shRNA has overhangs of 1 or 2 nucleotides. In certain embodiments, the siRNA agent that results from intracellular processing of the shRNA overhangs has two 3′ overhangs. In another embodiment, the overhangs are UU.

The agent that promotes protein expression can be an agent the results in increased levels of brain derived neurotrophic factor (BDNF) in the cell. For example, BDNF may be administered to the subject. Alternatively, or in addition to the agent that results in the increased levels of BDNF, the agent that promotes protein expression may be an antisense agent/molecule/oligonucleotide or an RNAi agent directed to at least one of HnmpK, PCBP1, or both.

The administering step of the method of modulating is effective at increasing or decreased the translation of an mRNA selected from Table 3 below. As a result, the method of modulating protein expression can be utilized to treat at least one symptom of a disease, disorder, or syndrome that is associated with overexpression (and/or accumulation) or underexpression of a protein translated from a mRNA found in Table 3.

In another aspect, the present disclosure provides a method of ameliorating, treating, or preventing at least one symptom of a disease, disorder, or syndrome associated with the overexpression or accumulation of a protein translated from an mRNA found in Table 3, such as DYRK1A and/or amyloid precursor protein (APP), in a cell (e.g., a neuron), the method comprising providing a subject in need thereof, and administering an effective amount of an agent that inhibits Mena translation, Mena transcription, or the association of Mena with the Mena-RNP complex, wherein the method is effective for ameliorating, treating, or preventing at least one symptom of a disease, disorder, or syndrome associated with the overexpression or accumulation of a protein translated from an mRNA found in Table 3, such as DYRK1A and/or amyloid precursor protein (APP).

As such, the present disclosure provides a method of ameliorating, treating, or preventing at least one symptom of a disease, disorder, or syndrome associated with the overexpression or accumulation of DYRK1A and/or amyloid precursor protein (APP) in a cell (such as a neuron), the method comprising providing a subject in need thereof, and administering an effective amount of an agent that inhibits Mena translation, Mena transcription, or the association of Mena with the Mena-RNP complex, wherein the method is effective for ameliorating, treating, or preventing at least one symptom of a disease, disorder, or syndrome associated with the overexpression or accumulation of DYRK1A and/or amyloid precursor protein (APP).

The disease, disorder, or syndrome that is associated with overexpression or accumulation of DYRK1A may be selected from the group consisting of a cognitive disorder, Down Syndrome, Alzheimer's disease, Parkinson's disease, or cancer.

In certain embodiments, the cancer is hematological malignancy or brain cancer.

In certain other embodiments, the cancer is breast cancer, pancreatic cancer, lung cancer, or colon cancer.

In further embodiments, the agent that inhibits protein expression is selected from an antisense agent/molecule/oligonucleotide, an RNAi agent/molecule/oligonucleotide, an antibody or an antigen binding fragment thereof, or a small molecule directed to Mena. For example, the agent that inhibits protein expression may be a peptide or pepido-mimetic that mimics and/or competes for Mena EVH1-ligand binding.

In a further aspect, the present disclosure provides a method of ameliorating, treating, or preventing at least one symptom of a disease, disorder, or syndrome associated with underexpression of a protein translated from a mRNA found in Table 3 (such as DYRK1A) in a cell (such as a neuron), the method comprising providing a subject in need thereof, and administering an effective amount of an agent that promotes protein expression by (i) inhibiting the expression of at least one of HnmpK, PCBP1, or both, or (ii) dissociating at least one of HnmpK, PCBP1, or both, from (or preventing the association of at least one of HnmpK, PCBP1, or both with) the Mena-RNP complex in the cell, wherein the method is effective for ameliorating, treating, or preventing at least one symptom of a disease, disorder, or syndrome associated with underexpression of the protein translated from a mRNA found in Table 3, such as DYRK1A.

Therefore, the present disclosure provides a method of ameliorating, treating, or preventing at least one symptom of a disease, disorder, or syndrome associated with underexpression of DYRK1A in a cell (e.g., a neuron), the method comprising providing a subject in need thereof, and administering an effective amount of an agent that promotes protein expression by (i) inhibiting the expression of at least one of HnmpK, PCBP1, or both, or (ii) dissociating at least one of HnmpK, PCBP1, or both, from (or preventing the association of at least one of HnmpK, PCBP1, or both with) the Mena-RNP complex in the cell, wherein the method is effective for ameliorating, treating, or preventing at least one symptom of a disease, disorder, or syndrome associated with underexpression of DYRK1A.

The agent that promotes protein expression can be an agent the results in increased levels of brain derived neurotrophic factor (BDNF) in the neuron. For example, the subject can be administered (i.e., intravenously administered) BDNF.

Alternatively, or in addition to the agent that increases BDNF levels, the agent that promotes protein expression can be an antisense oligonucleotide or an RNAi molecule directed to at least one of HnmpK, PCBP1, or both.

In any aspect or embodiments described herein, the subject is selected from the group consisting of a cell, a mammal, and a human.

In yet a further aspect, the present disclosure provides a method of diagnosing a subject as having a Mena-RNP complex associated disease, disorder, or syndrome the method comprising: obtaining or providing a sample from the subject; detecting the expression level of the protein in the sample from the subject; comparing the expression level in the sample to a control having normal expression levels of the protein; and diagnosing the subject as having a disease, disorder, or syndrome associated with the dysregulation of the expression of the protein when the sample has increased or decreased expression relative to the control, wherein the protein is at least one protein selected from Table 3.

In some embodiment, the method further comprises administering to the subject an agent that inhibits protein expression from the Mena-RNP complex or promotes protein expression from the Mena-RNP complex. In any aspect or embodiment of the present disclosure, the administering step may result in the modulation of the translation of an mRNA selected from Table 3. The subject is selected from the group consisting of a cell, a mammal, and a human.

The agent that inhibits protein expression from the Mena-RNP complex may do so by inhibiting Mena translation, Mena transcription, or the association of Mena with the Mena-RNP complex. For example, the agent that inhibits protein expression may be selected from an antisense agent, an RNAi agent, an antibody or an antigen binding fragment thereof, peptide or a small molecule directed to Mena. In particular embodiments, the agent that inhibits Mena translation, Mena transcription, or the association of Mena with the Mena-RNP complex inhibits expression of the protein in the cell.

The agent that promotes protein expression from the Mena-RNP complex may do so by (i) inhibiting the expression of at least one of HnmpK, PCBP1, or both, or (ii) dissociating at least one of HnmpK, PCBP1, or both, from (or preventing the association of at least one of HnmpK, PCBP1, or both with) the Mena-RNP complex in a cell. For example, the agent that promotes protein expression may be an agent the results in increased levels of brain derived neurotrophic factor (BDNF) in the cell, and/or an RNAi agent directed to at least one of HnmpK, PCBP1, or both.

In any aspect or embodiment of the present disclosure, the cell is a neuron.

Detecting the expression level of the protein (such as DYRK1A or APP) may comprise detecting the protein. For example, detecting the protein may be accomplished via at least one of immunohistochemistry, enzyme-linked immunosorbent assay, western blot, or a combination thereof.

Detecting the expression level of the protein (e.g., DYRK1A or APP) may comprise detecting mRNA of the protein, which may be accomplished via at least one of fluorescent in situ hybridization, northern blot, reverse-transcription polymerase chain reaction (RT-PCR), RT real time PCT, microarray, or a combination thereof.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure may be utilized in numerous combinations, all of which are expressly contemplated by the present disclosure. These additional advantages objects and embodiments are expressly included within the scope of the present disclosure. The publications and other materials used herein to illuminate the background of the invention, and in particular cases, to provide additional details respecting the practice, are incorporated by reference.

The practice of the present invention will employ conventional techniques of cell biology, cell culture, molecular biology, transgenic biology, microbiology, recombinant DNA, and immunology, which are within the skill of the art. Such techniques are described in the literature. See, for example, Molecular Cloning, A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Culture Of Animal Cells (R. I. Freshney, Alan R. Liss, Inc., 1987); B. Perbal, A Practical Guide To Molecular Cloning (1984); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

### EXAMPLES

Experimental Model and Subject Details

Animals. All experiments were performed according to the Guide for the Care and Use of Laboratory Animals and were approved by the National Institutes of Health, and the Committee on Animal Care at the Massachusetts Institute of Technology (Cambridge, Mass., USA). Female pregnant mice were euthanized with CO₂ and embryos were isolated and further dissected in 10 mM 4-(2-hydroxyethyl)-1-piperazineethanesulfonic acid (HEPES) and 1× Hank's Balanced Salt Solution (HBSS) (GIBCO/Invitrogen). Mice of the following strains were used: Swiss Webster, mixed background Mena+/+; VASP−/−; EVL−/−, Mena+/−; VASP−/−; EVL−/−, Mena−/−; VASP−/−; EVL−/− (mve), Mena+/+; VASP+/+; EVL+/+, and Mena−/−; VASP+/+; EVL+/+.

Primary Neuron Cultures. Cortical neurons from E15.5 mouse brains were plated on poly-D-lysine (PDL, SIGMA, St. Louis, Mo., USA) or PDL and Laminin (Southern Biotech, Birmingham, Ala., USA) and cultured for 2 days before treatments, unless otherwise indicated. Briefly, cortical tissue was dissected in 10 mM HEPES and 1× HBSS, washed and trypsinized in the same buffer for 15minutes at 37° C. Tissues were then washed in Dulbecco's Modified Eagle Medium (DMEM) 1× with 10% Fetal Bovine Serum (FBS) to inactivate trypsin, and triturated in the same medium. Following trituration neurons were pelleted at 600×g for 5 minutes, resuspended in serum-free Neurobasal medium (Invitrogen, Carlsbad, Calif., USA), supplemented with B27 (Gibco, Gaithersburg, Md., USA) and Penicillin/Streptomycin (Pen/Strep; Gibco, Gaithersburg, Md., USA), and plated on PDL-coated coverslips or petri dishes.

Cell Lines. MEFs and N2A cells were cultured at 37° C., 5% CO₂, in DMEM supplemented with 10% FBS and Pen/Strep.

Method Details

Primary neuron stimulation with BDNF. Before stimulations with Brain-Derived Neurotrophic Factor (BDNF; 50 ng/mL; R&D Systems, Minneapolis, Minn., USA), neurons were starved for 4 hours in L15-Leibowitz medium (Invitrogen, Carlsbad, Calif., USA), and to block translation, cells were incubated with 40 μM Anisomycin (SIGMA, St. Louis, Mo., USA) in L15, for 30 minutes prior to BDNF addition. BDNF was added for 15 minutes. Where needed, neurons were transfected using Amaxa Nucleofector mouse neuron kit (LONZA, Basel, Switzerland) according to the manufacturer's instructions. All experiments were repeated at least three times to eliminate technical and biological variations.

Primary Neurons on Transwell Filters/Axotomy. Cortical neurons from E15.5 mouse brains were plated on the top compartment of 6-well hanging inserts with 1 μm membrane pores (polyethylene terephthalate (PET); Millipore, Billerica, Mass., USA), coated on both sides of the membrane with PDL. The cells were cultured for 2 days in serum-free Neurobasal medium, supplemented with B27 and Pen/Strep. Prior to stimulation, neurons were starved as described above and the cell bodies were scraped from the top compartment of the filter, leaving the axons at the bottom. BDNF was added to the axons for 15 minutes and after stimulation, the bottom compartment was washed with ice cold phosphate buffered saline (PBS) and lysed for protein or mRNA extraction. For 30 minutes prior to BDNF addition, 40 μM Anisomycin in L15 was used for translational inhibition. All experiments were repeated at least three times, to minimize technical and biological variability.

siRNA in Primary Neurons. siRNA smartpools against HnrnpK, Pcbp1 and Safb2 were obtained from Dharmacon (Lafayette, Colo., USA) and introduced in neurons with Amaxa Nucleofection (LONZA, Basel, Switzerland), as per the manufacturer's instructions. A green fluorescent protein (GFP) control plasmid provided with the mouse neuron nucleofection kit was co-transfected to visualize cells with the siRNAs. The knockdown efficiency was assessed by immunofluorescence (IF) and microscopy.

Immunofluorescence (IF). Coverslips were fixed for 20 minutes at 37° C. with 4% paraformaldehyde (PFA) in PHEM buffer (120 mM Sucrose, 2 mM MgCl₂, 10 mM EDTA, 25 mM HEPES, 60 mM PIPES), rinsed with PBS and then permeabilized with 0.3% Triton-X100 in PBS for 5 minutes at room temperature. Blocking for 1 hour in 10% serum in PBS was followed by incubation with primary antibodies diluted in blocking solution for 1 hour at room temperature. After PBS rinses, secondary antibodies were added to the coverslips, diluted in blocking solution, for 45 minutes at room temperature. Phalloidin staining of F-actin was performed for 30 minutes at room temperature, followed by PBS rinses, and mounting of the coverslips on slides with Fluoromount-G (Southern Biotech, Birmingham, Ala., USA) for imaging. Primary antibodies used: Ms-Anti-HnrnpK (1:50, SantaCruz, Dallas, Tex., USA) ms-anti-Mena (1:500) (Lebrand et al., 2004), Rb-anti-Mena (1:500) and Rb-anti-VASP (1:500) generated in the Gertler laboratory. All secondary antibodies used were from Jackson Laboratories (Bar Harbor, Me., USA), conjugated to −405, −488, −595, or −647 fluorophores and diluted 1:500.

RNA Fluorescent In Situ Hybridization (FISH). RNA FISH was performed using custom Stellaris® FISH probes (LGC Biosearch Technologies, Novato, Calif., USA), according to the manufacturer's protocol. Briefly, cells on coverslips were fixed with 4% paraformaldehyde in PBS 1× for 15 minutes at 37° C., and subsequently permeabilized with 0.3% Triton-X100 in PBS for 5 minutes at room temperature. Coverslips were washed in 10% deionized Formamide, 2× Saline-sodium Citrate (SSC) (wash buffer) for 5 minutes at room temperature and then hybridized in 10% formamide, 2×SSC, 10% Dextran sulfate, 0.5 μg/mL Salmon Sperm DNA, 1 mg/mL yeast tRNA, 1% bovine serum albumin (BSA), and 125 nM of RNA probe, in a dark humidified chamber at 37° C. O/N. After hybridization the coverslips were washed in wash buffer at 37° C. for 30 minutes in the dark. Wherever IF was performed along with the FISH, the primary antibodies were diluted in the hybridization buffer with the probe and incubated simultaneously, and the secondary antibodies were added to the post-hybridization wash (30 minutes at 37° C.). After the post-hybridization wash, coverslips were incubated with phalloidin in PBS, 30 minutes at room temperature, rinsed in PBS and mounted on slides with Fluoromount-G (Southern Biotech, Birmingham, Ala., USA) for imaging. For the unmasking experiments, neurons were incubated with pepsin for 30 seconds after fixation, as previously described (Buxbaum et al., 2014). For the custom probes, the entire mRNA sequence of mouse and human dyrk1a was used on the Stellaris® website. All experiments were repeated at least three times, and a minimum of 10 neurons per condition per experiment was imaged and used for quantifications.

Immunoprecipitation (IP). Cortical neurons cultured for three days in vitro were lysed in lysis buffer (20 mM Tris ph 8.0, 200 mM NaCl, 2 mM MgCl2, 10% glycerol, 1% NP-40) supplemented with Roche (Basel, Switzerland) complete EDTA-free protease and phosphatase inhibitor tablets on ice for 20 minutes. For whole brain lysate, E15.5 mouse brains were homogenized in lysis buffer using a Dounce homogenizer chilled on ice. Collected lysates were cleared by centrifugation for 20 minutes 14k rpm at 4° C., and incubated overnight at 4° C. with antibodies on magnetic protein G beads (incubated in PBS for 4 hours at 4° C. After IP, beads were washed three times with lysis buffer containing 0.4% NP40, and boiled in 2× sodium dodecyl sulfate (SDS) sample buffer for loading onto an acrylamide gel either for western blotting or for Mass Spectrometry.

HITS-CLIP Modification. E15.5 mouse brains were dissected, rinsed and triturated in PBS and UV-irradiated three times at 400 mJ/cm² in a Stratalinker (254 nm). The tissue suspension was collected by centrifugation and the pellet was lysed in 20 mM Tris ph 8.0, 200 mM NaCl, 2 mM MgCl₂, 10% glycerol, 1% NP-40) supplemented with Roche complete EDTA-free protease and phosphatase inhibitor tablets. The lysate was subsequently treated with DNAseI (New England BioLabs®, Ipswich, Mass., USA, M0303L) and RNAseIF (New England BioLabs®, Ipswich, Mass., USA, M0243L) at 37° C. and centrifuged for 10 minutes at 13000 rpm. The cleared lysates were then used for Mena IP. Following the IP, the samples were washed stringently three times with 1M NaCl in lysis/IP buffer and the beads were collected in TRIZOL Reagent for RNA extraction, according to the manufacturer's instructions. Purified RNA was subsequently used for the construction of libraries and sequencing on an ILLUMINA Platform (miSeq; San Diego, Calif., USA). The experiment was repeated twice, using two biological replicates per sample, per experiment, to eliminate technical and biological variability. Only the mRNAs that had more than 10 reads and 3-fold enrichment between the Mena and control IP samples were considered significant for subsequent analysis.

Oligo dT capture. E15.5 mouse brain lysates were prepared as described above for HITS-CLIP samples. The lysates were then treated with DNase for 5 minutes at 37° C. and centrifuged 20 minutes at 14000 rpm at 4° C. Half of each lysate was treated with 1 mg/mL RNaseA for 15 minutes at 37° C. and all of the samples were finally heated at 65° C. for 5 minutes and kept on ice. OligodT Dynabeads (Pierce) were added to lysates for 12 minutes at room temperature and pelleted on a magnet. Following incubation, the beads were washed three times in 1M NaCl lysis buffer and 2× Laemli Buffer was used for Western blot analysis of each sample.

mRNA Pulldown Assay. The sequence of the 3′UTR of the dyrk1a and lhx6 mRNA was cloned in a pBS KS vector and linearized. In vitro transcription was carried out on the linearized templates, using the Ampliscribe T7-Flash Biotin-RNA Transcription Kit, according to the manufacturer's instructions (Epicentre®, Madison, Wis., USA), in order to generate biotinylated probes for the 3′UTR of dyrk1a mRNA. λ-phage DNA and the 3′UTR of the lhx6 gene was also used to generate a control, non-specific biotinylated RNA probe, by in vitro transcription. Following precipitation and reconstitution in H₂O, the biotinylated probes were captured on Streptavidin Dynabeads (My Streptavidin T1 beads, Pierce, Waltham, Mass., USA) for 1 hour at room temperature E15.5 brains lysed in 20 mM Tris ph 8.0, 200 mM NaCl, 2 mM MgCl₂, 10% glycerol, 1% NP-40 supplemented with Roche complete EDTA-free protease and phosphatase inhibitor tablets and RNAse Inhibitors (Ambion) were incubated with the beads, O/N at 4° C. The beads were subsequently washed in lysis buffer and processed for western blot analysis.

Western Blot. Protein samples were resolved by SDS-PAGE, transferred to nitrocellulose membranes and immunoblotted. Blocking was performed for 1 hour with 3% BSA in PBS at room temperature, and then the membranes were incubated with primary antibodies in PBS+0.1% Tween-20, O/N at 4° C. After thorough washes, the membranes were incubated with secondary HRP-conjugated antibodies at 1:5000 dilutions and they were visualized by enhanced chemiluminescence (SuperSignal West Pico Chemluminescent HRP substrate; ThermoFisher, Waltham, Mass., USA). Alternatively, fluorescent LICOR secondary antibodies were used at 1:10000, and the membranes were imaged. Primary antibodies used: Rb-anti-PCBP1 (Abcam, Cambridge, United Kingdom) 1:1000; Rb-anti-HnrnpK (Cell Signaling, Beverly, Mass., USA) 1:1000; Ms-anti-SafB2 (Abcam, Cambridge, United Kingdom) 1:2000; Ms-anti-Dyrk1a (Abnova) 1:1000; Rb-anti-FMR1 (Cell Signaling, Beverly, Mass., USA) 1:1000; Ms-anti-beta Catenin (BD Biosciences, San Jose, Calif., USA) 1:2000; Rb-anti-HnrnpM (Bethyl Laboratories, Montgomery, Tex., USA) 1:1000; Rb-anti-HnrnpA2B1 (Elabscience, Wuhan, China) 1:1000; Rb-anti-RRBP1 (Bethyl Laboratories, Montgomery, Tex., USA) 1:1000; Ms-anti-MBNL1 (Wolfson Centre for Inherited Neuromuscular Disease, Oswestry, United Kingdom) 1:1000; Ms-anti-tubulin (DM1A) 1:10000

Mass Spectrometry. Two technical replicates of this experiment were performed, each replicate used two independent biological replicates for Mena IP from wild type brains, and two replicates for Mena IP from Mena-null brains (as a negative control for specificity). During IP for mass spectrometry the anti-Mena antibody (clone A351F7D9) was covalently crosslinked with DMP to protein G magnetic beads. Acrylamide gels were stained with Coomasie Brilliant Blue. After destaining with 40% ethanol/10% acetic acid, proteins were reduced with 20 mM dithiothreitol (SIGMA, St. Louis, Mo., USA) for 1 hour at 56° C. and then alkylated with 60 mM iodoacetamide (SIGMA, St. Louis, Mo., USA) for 1 hour at 25° C. in the dark. Proteins were then digested with 12.5 ng/μL modified trypsin (Promega, Madison, Wis., USA) in 50 μl of 100 mM ammonium bicarbonate, pH 8.9 at 25° C., overnight. Peptides were extracted by incubating the gel pieces with 50% acetonitrile/5% formic acid, then 100 mM ammonium bicarbonate, repeated twice followed by incubating the gel pieces with 100% acetonitrile, then 100 mM ammonium bicarbonate, repeated twice. Each fraction was collected, combined, and reduced to near dryness in a vacuum centrifuge. Per the manufacturer's instructions, each sample was labeled with a unique iTRAQ 4plex (AB Sciex, Framingham, Mass., USA). Following a 1 hour incubation, all samples were combined and concentrated to completion. The combined labeled peptides were desalted using Protea C18 spin tips and resuspended in 0.1% formic acid. Peptides were separated by reverse phase HPLC using an EASY-nLC1000 (Thermo, Waltham, Mass., USA) over a 140-minute gradient before nanoelectrospray using a QExactive mass spectrometer (Thermo, Waltham, Mass., USA). Mass spectrometry data were analyzed using Mascot (Matrix Science, Boston, Mass., USA) and Proteome Discoverer (Thermo, Waltham, Mass., USA).

RT-PCR and Quantitative PCR. cDNA synthesis was performed using the Invitrogen Superscript III First Strand Synthesis for RT-PCR kit (Carlsbad, Calif., USA), with Random Hexamer primers, according to the manufacturer's instructions. Quantitative PCR was performed using the Biorad iQ SYBR Green Supermix on a CFX96 Real Time PCR Detection System, with the following gene-specific primers:

| | |
|---|---|
| mouse GAPDH | 5′-catgttccagtatgactccactc; |
| mouse GAPDH | 3′-ggcctcaccccatttgatgt |
| mouse Mena | 5′-gggcagaaagattcaagacc; |
| mouse Mena | 3′-gcgaagacattggcatcc |
| mouse Dyrk1a | 5′-caaacggagtgcaatcaaga; |
| mouse Dyrk1a | 3′-agcacctctggagaccgata |
| mouse Robo1 | 5′-catcaagaggatcagggagc; |
| mouse Robo1 | 3′-ggttgtcttcagctttcagtttc |
| mouse Elavl1 | 5′-agccaatcccaaccagaac; |
| mouse Elavl1 | 3′-acaccagaaatcccactcatg |
| mouse β-Ctnn | 5′-ctatcccagaggctttatccaag; |
| mouse β-Ctnn | 3′-ccagagtgaaaagaacggtagc |
| mouse Khsrp | 5′-gccaatcagactacaccaagg; |
| mouse Khsrp | 3′-gccacttgtgttgcttcttg |
| mouse Eif4ebp2 | 5′-ccatctgcccaatatccctg; |
| mouse Eif4ebp2 | 3′-tgtccatctcaaactgagcc |
| mouse Vamp2 | 5′-aagttgtcggagctggatg; |
| mouse Vamp2 | 3′-cgcagatcactcccaagatg |

Imaging. Imaging was performed using a Deltavision microscope (Applied Precision, Issaquah, Wash., USA), with a Coolsnap HQ camera (Photometrics, Tuscon, Ariz., USA); post-acquisition image processing was performed using SoftWoRx v.XX (Applied Precision, Issaquah, Wash., USA). Maximum intensity projections of 2-4 optical sections were generated using ImageJ. Only growth cones that were not in contact with other cells or processes and had extended more than 0.3 mm away from the cell body were chosen for imaging. All fluorescence quantitation used original unprocessed image data, with no pixels at zero intensity or saturated. In panels displayed in the figures, for consistent visibility across the intensity range, contrast and brightness were adjusted uniformly within each experimental series.

Quantification and Statistical Analysis

All microscopy experiments were repeated at least three times with different biological samples, and at least ten axons were analyzed per condition, per experiment. For colocalization studies, the JaCoP plugin of ImageJ was used to calculate Pearson's Coefficient Corellation. All biochemical assays were performed at least three times with two biological replicates each time per sample, to minimize variability. Statistical significance was assessed either in Excel, or Graphpad Prism6 (La Jolla, Calif., USA), using Student's t test, non-parametric, or two-way ANOVA, specified in Figure legends for each experiment. All graphs represent mean values±StDev.

TABLE 1
Key Resources for the Examples

| | | |
|---|---|---|
| REAGENT or RESOURCE | SOURCE | IDENTIFIER |

Antibodies

| | | |
|---|---|---|
| Rb-anti-Pcbp1 | Abcam | ab168378 |
| 1:1000 (WB) | | |
| Ms-anti Pcbp1 | Santa Cruz | sc-393076 |
| 1:200 (IF) | | |
| Rb-anti-HnrnpK | Cell | 4675 |
| 1:1000 (WB) | Signaling | |
| Ms-anti HnrnpK | Santa Cruz | sc-28380 |
| 1:50 (IF) | | |
| Ms-anti-SafB2 | Abcam | ab67344 |
| 1:2000 (WB) | | |
| Ms-anti-Dyrk1a | Abnova | H00001859-M01 |
| 1:1000 (WB); | | |
| 1:200 (IF) | | |
| Rb-anti-FMR1 | Cell | 4317 |
| 1:1000 (WB) | Signaling | |
| Ms-anti-β | BD | 610153 |
| Catenin 1:2000 | Biosciences | |
| (WB) | | |
| Rb-anti-HnrnpM | Bethyl | A303.910A |
| 1:1000 (WB) | Laboratories | |
| Rb-anti- | Elabscience | EAP0297 |
| HnrnpA2B1 | | |
| 1:1000 (WB) | | |
| Rb-anti-RRBP1 | Bethyl | |
| 1:1000 (WB) | Laboratories | |
| Ms-anti-MBNL1 | E. T. Wang | N/A |
| 1:1000 (WB) | | |
| Rb-anti-GapdH | Cell | 2118 |
| 1:2500 (WB) | Signaling | |
| Ms-anti-Tubulin | Sigma- | T9026 |
| (DM1A) 1:20000 | Aldrich | |
| (WB) | | |
| Ms-anti-Mena | (Lebrand et | N/A |
| 1:500 (IF); | al., 2004) | |
| 1:5000 (WB) | | |
| Rb-anti-Mena | (Gertler et | N/A |
| 1:250 (IF); | al., 1996) | |
| 1:5000 (WB) | | |
| Rb-anti-VASP | (Lanier et | N/A |
| 1:500 (IF); | al., 1999) | |
| 1:5000 (WB) | | |
| Rb-anti-Tbr1 | Chemicon | AB9616 |
| 1:1000 (WB) | | |
| Rb-anti-Map2 | Chemicon | AB5622 |
| 1:1000 (WB) | | |
| Ms-anti-panTau | Chemicon | MAB3420 |
| 1:1000 (WB) | | |
| Ms-anti-Elavl1 | Santa Cruz | sc-5261 |
| (HuR) 1:1000 | | |
| (WB) | | |

Critical Commercial Assays

| | | |
|---|---|---|
| Ampliscribe | Epicentre | ASB71110 |
| T7-Flash | | |
| Biotin-RNA | | |
| Transcription | | |
| Kit | | |

Experimental Models: Cell Lines

| | | |
|---|---|---|
| N2A cells | ATCC | CCL-131 |
| MEFs | (Bear et | N/A |
| | al., 2000) | |

Experimental Models: Organisms/Strains

| | | |
|---|---|---|
| Mouse: | Taconic | https:// |
| Swiss webster | | www.taconic.co |
| | | m/mouse-model/ |
| | | swiss-webster |
| Mouse: Mena | | (Kwiatkowski et |
| | | al., 2007) |
| Mouse: mve | | (McConnell et |
| | | al., 2016) |

qPCR primer sets

| | | |
|---|---|---|
| mouse GAPDH 5′- | IDT | N/A |
| catgttccagtatga | | |
| ctccactc | | |
| mouse GAPDH | | |
| 3′-ggcctcacccca | | |
| tttgatgt | | |
| mouse Mena 5′- | IDT | N/A |
| gggcagaaagattca | | |
| agacc | | |
| mouse Mena 3′- | | |
| gcgaagacattggca | | |
| tcc | | |
| mouse Dyrk1a | IDT | N/A |
| 5′-caaacggagtgc | | |
| aatcaaga | | |
| mouse Dyrk1a | | |
| 3′-agcacctctgga | | |
| gaccgata | | |
| mouse Robo1 5′- | IDT | N/A |
| catcaagaggatcag | | |
| ggagc | | |
| mouse Robo1 3′- | | |
| ggttgtcttcagctt | | |
| tcagtttc | | |
| mouse Elavl1 | IDT | N/A |
| 5′-agccaatcccaa | | |
| ccagaac | | |
| mouse Elavl1 | | |
| 3′-acaccagaaatc | | |
| ccactcatg | | |
| mouse β-Ctnn | IDT | N/A |
| 5′-ctatcccagagg | | |
| ctttatccaag | | |
| mouse β-Ctnn | | |
| 3′-ccagagtgaaaa | | |
| gaacggtagc | | |
| mouse Khsrp | IDT | N/A |
| 5′-gccaatcagact | | |
| acaccaagg | | |
| mouse Khsrp | | |
| 3′-gccacttgtgtt | | |
| gcttcttg | | |
| mouse Eif4ebp2 | IDT | N/A |
| 5′-ccatctgcccaa | | |
| tatccctg | | |
| mouse Eif4ebp2 | | |
| 3′-tgtccatctcaa | | |
| actgagcc | | |
| mouse Vamp2 5′- | IDT | N/A |
| aagttgtcggagctg | | |
| gatg | | |
| mouse Vamp2 3′- | | |
| cgcagatcactccca | | |
| agatg | | |

Recombinant DNA

| | |
|---|---|
| mCherry or GFP | (Bear et |
| FP4-mito | al., 2000) |
| contrsuct | |
| mCherry or GFP | (Bear et |
| AP4-mito | al., 2000) |
| construct | |

siRNA

| | | |
|---|---|---|
| Smartpool ON | Dharmacon | L-048002- |
| TARGET Plus | | 01-0005 |
| HnrnpK siRNA | | |
| Smartpool ON | Dharmacon | L-062816- |
| TARGET Plus | | 01-0005 |
| Pcbp1 siRNA | | |
| Smartpool ON | Dharmacon | L-054890- |
| TARGET Plus | | 01-0005 |
| Safb2 siRNA | | |
| Smartpool ON | Dharmacon | D-001810-10-20 |
| TARGET Plus | | |
| non-targeting | | |
| pool siRNA | | |

Software and Algorithms

| | | |
|---|---|---|
| Graphpad | Graphpad | 6 |
| Prism 6 | Software | |
| ImageJ | ImageJ | |
| Adobe | Adobe | CS5 |
| Illustrator | | |
| CS5 | | |
| R custom | This study | N/A |
| script | | |
| RBPmap | (Paz et al., | http:// |
| | 2010) | rbpmap.technion. |
| | | ac.il/ |

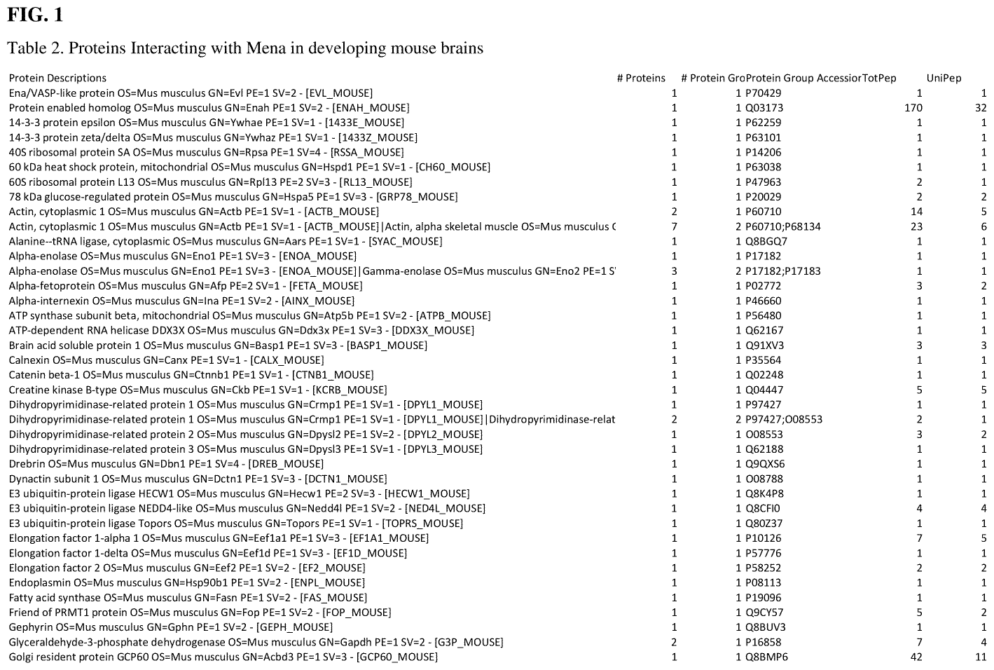

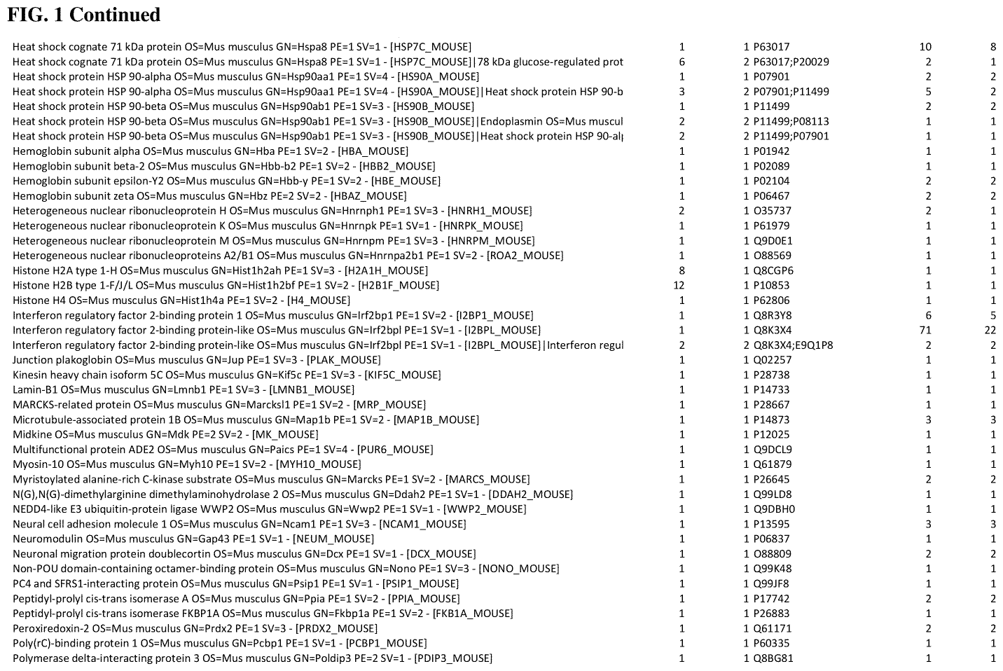

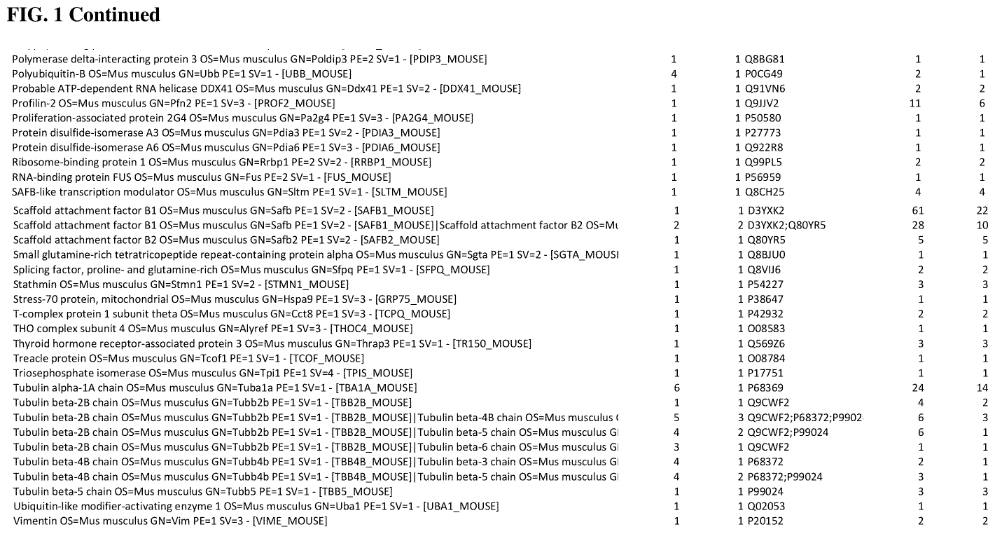

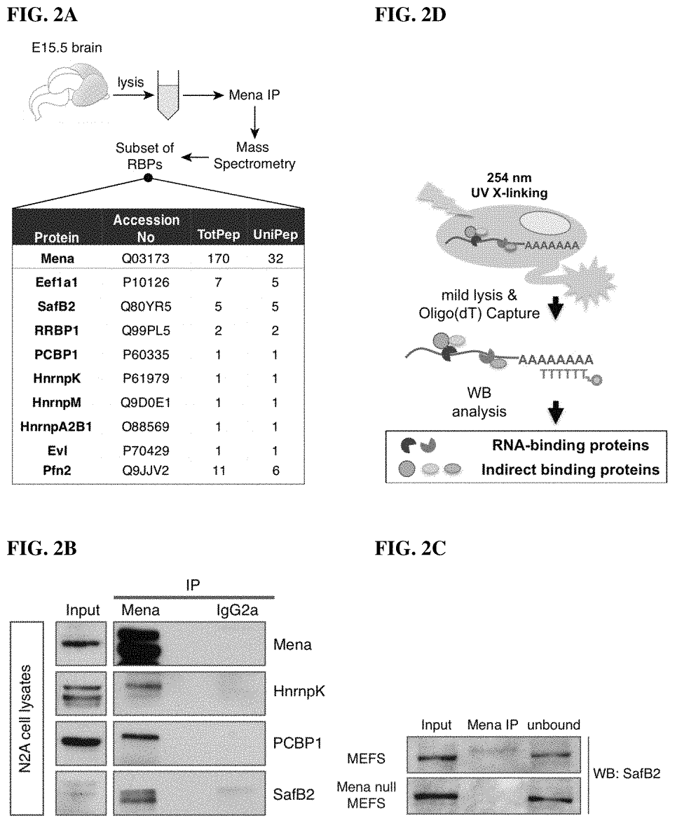

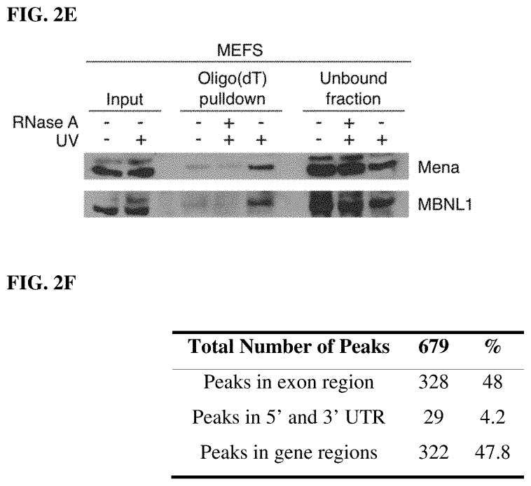

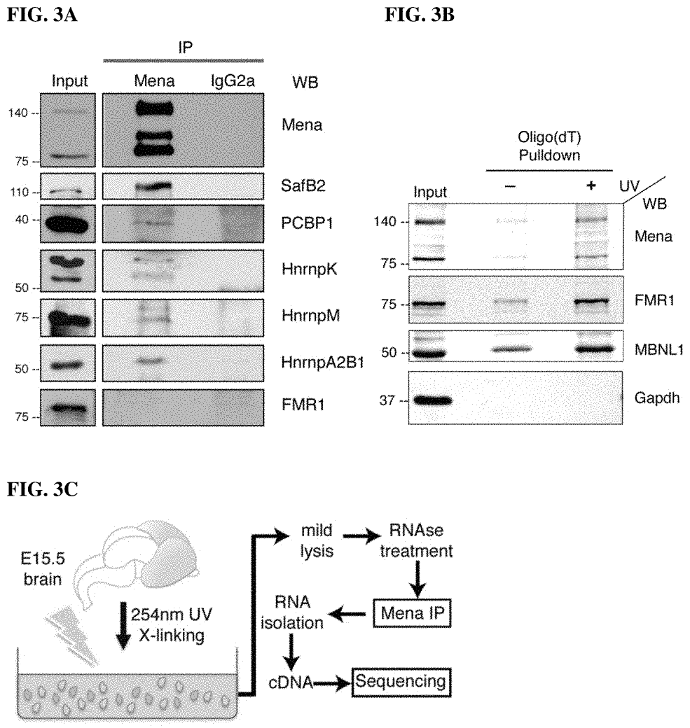

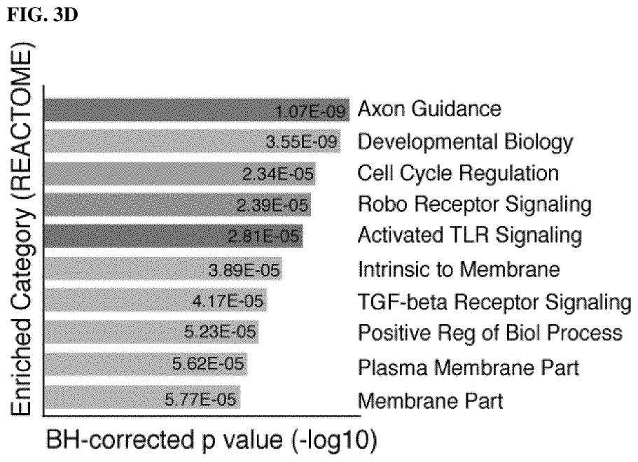

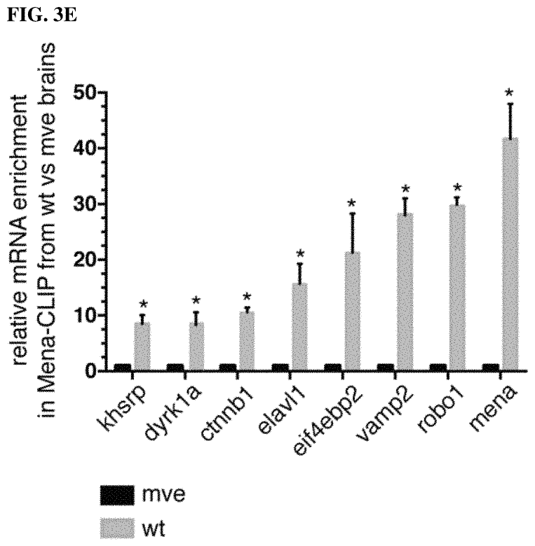

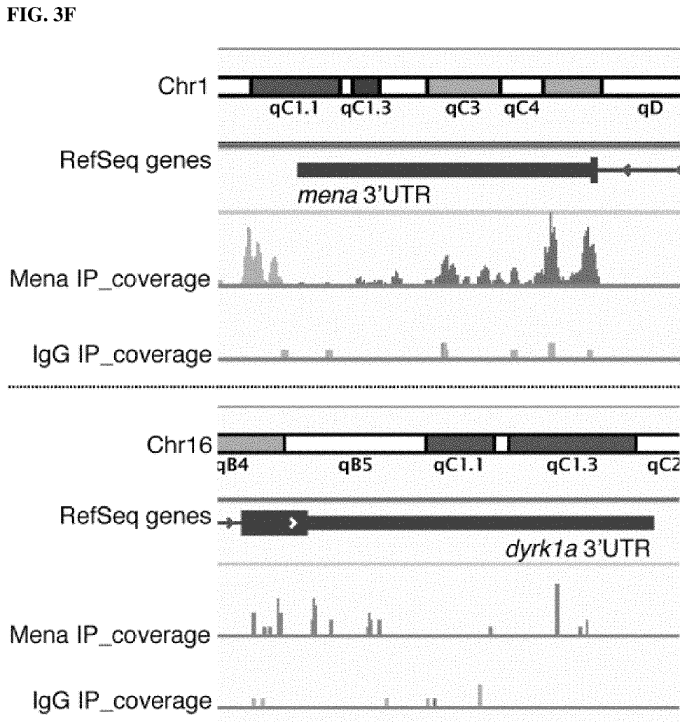

Novel Interactions of Mena with Multiple RNA Binding Proteins in the Developing Brain. To gain insight into the mechanisms underlying Mena function beyond its established role in actin polymerization (Bear and Gertler, 2009) and in integrin-mediated signaling (Dent et al., 2007; Gupton et al., 2012; Gupton and Gertler, 2010) in the developing NS, the inventors sought to identify its interactome. Mass spectrometry was performed after immunoprecipitation (IP) of Mena from lysates of E15.5 mouse brains from Mena-wild type and Mena-deficient animals. Aside from known Mena binding partners, including Profilin 2 and EVL (Barzik et al., 2005; Giesemann et al., 2003), additional Mena-associated proteins were identified (Table 2; see FIG. 1). Surprisingly, a significant number of RNA-binding proteins, including translation factors and mRNA transport proteins, were identified in the Mena IP (FIG. 2A). To verify the specificity of these interactions, co-IP experiments from E15.5 mouse brains was performed using Mena-deficient brain lysates or isotype-specific antibodies as negative controls. Several RNA-binding proteins (RBPs) identified in the Mass Spectrometry analysis co-IPed specifically with Mena from developing brain lysates, while FMR1, an RBP not identified by the proteomic interactions analysis was not detected in the co-IP (FIG. 3A; the multiple bands that appear on the Mena WB, correspond to different Mena protein isoforms expressed in neurons (Gertler et al., 1996; Lanier et al., 1999)). Several of the Mena-RBP interactions were also detected in N2A neuroblastoma cells and in mouse embryonic fibroblasts (MEFs) by co-IP (FIGS. 2B and 2C). Interestingly, RNase treatment of brain lysates did not affect the recovery of RBPs in Mena co-IPs (data not shown), indicating that the Mena:RBP interactions identified herein do not depend on the presence of RNA.

Mena Associates with Cytosolic mRNAs In Vivo. Given that Mena associated with multiple RBPs in developing brain lysates, we wondered whether Mena-containing complexes were also associated with mRNA. To test this hypothesis, we used Oligo(dT) pulldown assays to capture polyadenylated mRNAs and mRNA-associated proteins from lysates prepared from brain tissue that was UV-crosslinked to preserve RNA-protein complexes (FIG. 2D and FIG. 3B). Western blot analysis of the captured proteins indicated that Mena was associated with mRNA (FIG. 3B). To test whether this association is neuronal specific, we performed additional Oligo(dT) pulldown assays and found that in MEFs Mena is also in complex with cytosolic mRNAs (FIG. 2E).

Identification of mRNAs Associated with Mena in the Brain. Mena can bind directly to a number of proteins, including ligands for its EVH1 (Ena/VASP Homology-1) domain, actin through its EVH2 domain and α5 integrin through its LERER domain (Drees and Gertler, 2008; Krause et al., 2003; Lanier and Gertler, 2000; Menzies et al., 2004), but lacks any known RNA binding sites (Bear and Gertler, 2009; Drees and Gertler, 2008; Gertler et al., 1996), raising the possibility that it associates with mRNA indirectly via one or more of the associated RBPs identified by mass spectrometry. To investigate this possibility, and to identify mRNAs in complex with Mena in neurons, immunoprecipitation was performed after crosslinking (CLIP assay). UV-crosslinking was used to preserve RNA-protein complexes in E15.5 mouse brain tissues, followed by lysate preparation and Mena IP. The stringent lysis conditions typically used for CLIP diminished recovery of RNA associated with Mena by co-IP (data not shown), suggesting that the association of Mena with mRNA may be indirect. A modified CLIP protocol with mild lysis and IP conditions improved mRNA recovery in the Mena IP and the associated mRNA was extracted from the beads, purified and processed for sequencing (FIG. 3C: modified High-throughput RNA Sequencing after CLIP; HITS-CLIP, see Star Methods) (Darnell, 2010; Licatalosi et al., 2008). Binding peaks were identified by the presence of multiple sequence reads in the sample that exhibited more than 10 reads and that were at least 3-fold enriched in the Mena vs. control CLIP samples (Table 3, below). The majority of the peaks were distributed within exons (48%) or gene regions (47.8%), while a small number of peaks mapped to the 5′ and 3′ UTRs of mRNAs (4.2%) (FIG. 2F and Table 3).

TABLE 3
mRNAs associated with Mena in developing mouse brains

1110051M20Rik
2400003C14Rik
2610002M06Rik
2610200G18Rik
2700081O15Rik
4921506J03Rik
9830001H06Rik
A730008H23Rik
Adcyap1r1
Aff4
AK140446
Amigo3
App
Arfgap2
Atp1a2
Atp2b1
Atp8a1
Bat2d
Bat2l2
Bmpr1a
Bsn
Btbd14b
Caprin1
Cav1
Cd24a
Cdk4
Cdk5r2
Cds2
Ceacam2
Celsr3
Cfl1
Chd6
Chmp2a
Cog8
Col1a1
Col5a1
Ctnnb1
Ctnnd1
Ctsa
Dcaf8
Dcakd
Ddx21
Dnmt3a
Dpm1
Dyrk1a
Egfl7
Eif4ebp2
Elavl1
Elavl3
Eme2
Enah
Enc1
Fam166b
Fam181b
Fam38b
Fam57a
Fut11
G3bp2
G6pdx
Galnt2
Gas7
Gemin4
Gm9934
Gmppb
Gnas
Grlf1
Gtf2i
Hjurp
Hnrnpc
Hnrnpul1
Hook3
Hp1bp3
Hspa8
Hspg2
Iglon5
Jund
Kcnc1
Kctdl2
Khsrp
Kidins220
Kif24
Klf13
Ktn1
Lass6
Ldlrad2
Lemd2
Lemd3
Lonp1
Lonrf2
Lrrc16a
Lrrc41
Mab2111
Maged1
Map3k7
Marcks
Mccc1
Medl21
mFLJ00163
Mll1
Mll3
Mn1
Mrps34
mt-Nd4
Mus81
Mybbp1a
Myh10
Myh9
Myo18a
Myo5a
Myo9b
Ncam1
Nktr
Nme3
Nr2f6
Nrp2
Ntn1
Ocel1
P2ry14
Pdf
Pea15a
Pik3ca
Pitpnc1
Pja2
Pkd113
Plk2
Pltp
Pomgnt1
Ppp2ca
Prrc2c
Psd3
Pten
Rab11b
Rab6b
Rad541
Ralyl
Rasa1
Reep5
Rnf123
Robo1
Rusc2
Sec24c
Sfrs6
Shank2
Slc1a2
Slc7a4
Slc8a1
Smc1a
Smg7
Snx32
Sorbs2
Sparc
Spnb2
Spsb3
Stmn3
Taok3
Tbc1d16
Tm9sf3
Tmcc2
Tnrc18
Tnrc6b
Top1
Traf3
Trank1
Trim28
Trp53inp2
Tspan31
Tuba1a
Tubb3
Tulp4
Ubap1
Ugt1a6b
Usp42
Vamp2
Vps4a
Vsig1
Zcchc14
Zfhx4
Zfml
Zfp354c
Zfp462
Zfp469

To identify potential biological processes controlled by the Mena-associated mRNAs, Gene Set Enrichment Analysis (Reactome) was performed using the Broad Institute platform (software.broadinstitute.org/gsea/index.jsp) (FIG. 3D and Table 3). Several of the most enriched gene sets represented processes that involve Mena function (e.g. Axon Guidance, Robo signaling, etc) (FIG. 3D). To confirm the specific association of selected mRNAs within each category with Mena, quantitative RT-PCR was performed after Mena CLIP assays from control and Mena-null E15.5 brains (referred to as mve throughout the text) (FIG. 3E). Interestingly, some of the Mena-complex associated mRNAs encode proteins that have been functionally linked to Mena in other studies (e.g. Vamp2 (Gupton and Gertler, 2010), Robol (Bashaw et al., 2000; McConnell et al., 2016; Yu et al., 2002), Ctnnbl (Najafov et al., 2012)), while others represent processes not previously associated with Mena (e.g. Khsrp, Elav11, Eif4ebp2, Dyrk1a, etc). Two of the most prevalent mRNAs identified by sequencing were those of dyrk1a and mena itself, both demonstrating multiple sequencing peaks in their gene region and in their 3′UTR (FIG. 3F).

Together, these data indicate that Mena can indirectly associate with specific mRNAs via its interacting RBPs, in a novel ribonucleoprotein (RNP) complex in developing neurons.

Mena Associates with the mRNA of dvrkla in Neurons. The mRNA of dyrk1a encodes a dual specificity kinase that has multiple functions in the NS (Barallobre et al., 2014; Hammerle et al., 2003; Hammerle et al., 2008; Tejedor and Hammerle, 2011). Dyrk1a inhibitors are being tested in Alzheimer's disease treatment (Coutadeur et al., 2015; Janel et al., 2014), whereas in models of Parkinson's disease Dyrk1a acts as a dopaminergic neuron survival factor (Barallobre et al., 2014). Moreover, recently Dyrk1a has been implicated in cases of autism and intellectual disability (Krumm et al., 2014; O'Roak et al., 2012; van Bon et al., 2015), and a Dyrk1a dosage-dependent role has been correlated with Down Syndrome etiology and pathology (Hammerle et al., 2003; Hammerle et al., 2008; Tejedor and Hammerle, 2011). The RNAseq data contained multiple reads that were consistent with an interaction between the Mena complex and the 3′UTR of dyrk1a message (FIG. 3F), and it has been shown that interactions of RBPs with 3′UTRs can regulate cytosolic mRNA localization and translation (Szostak and Gebauer, 2013). Thus, Mena may be important for dyrk1a mRNA dynamics in neurons.

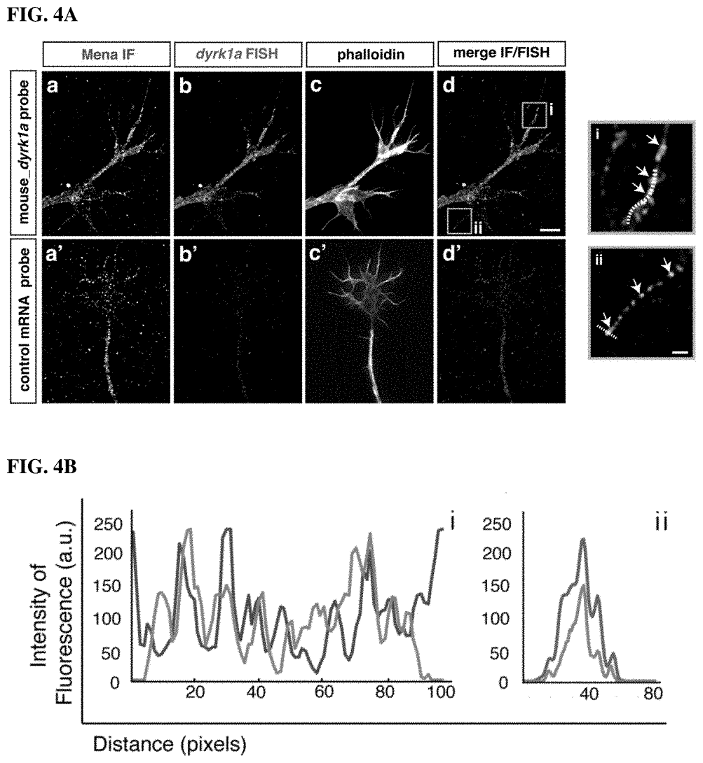

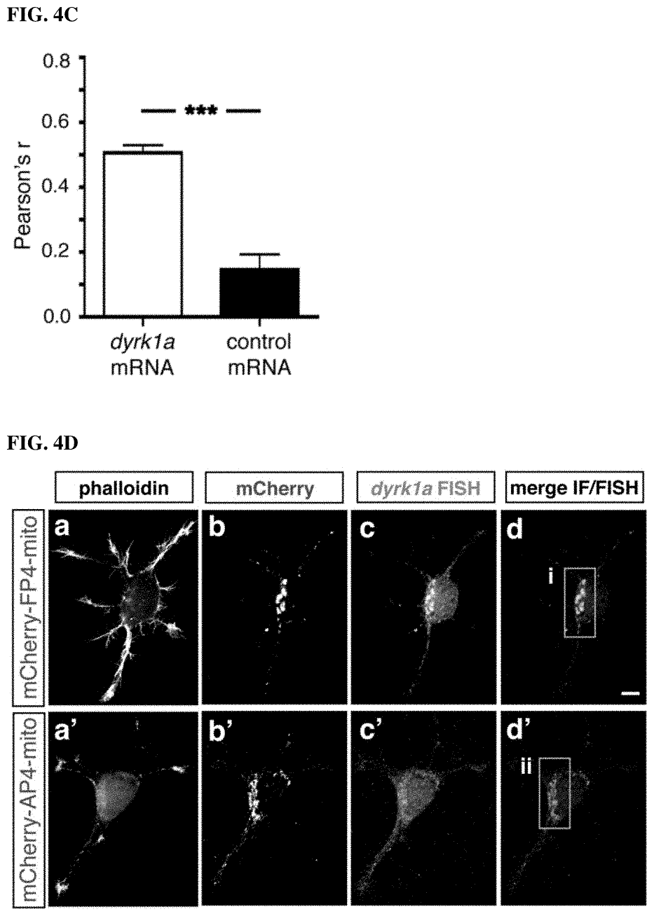

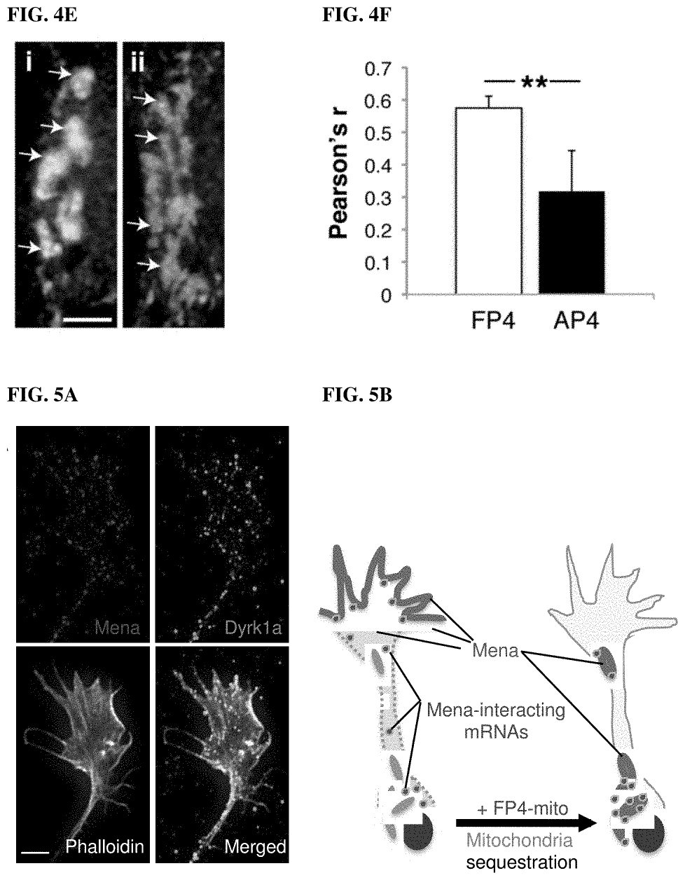

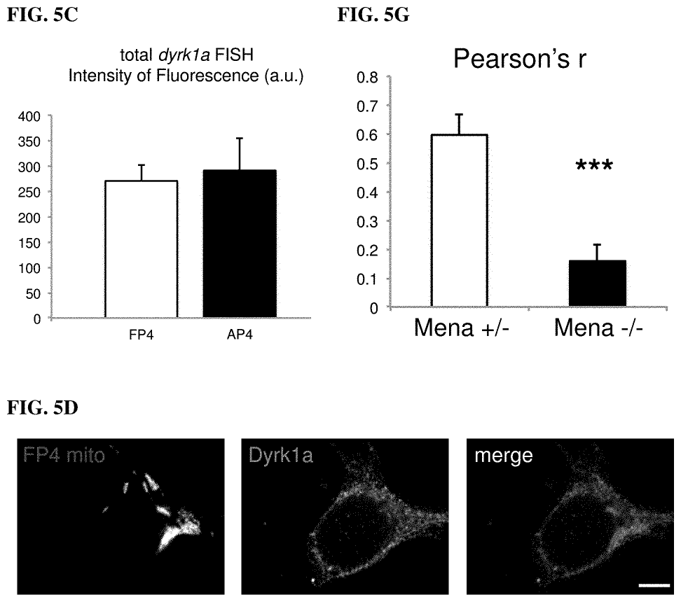

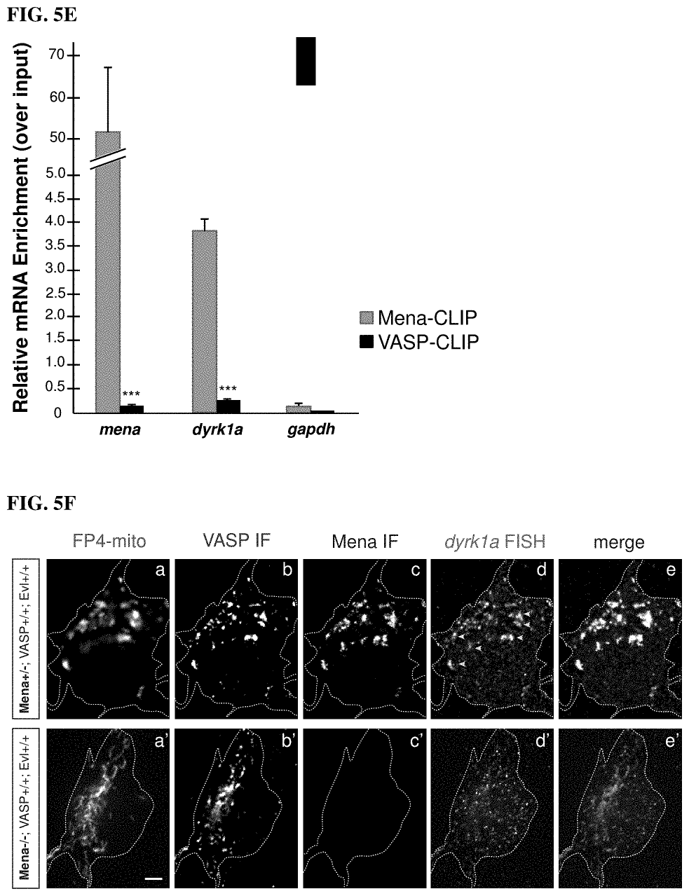

The specific localization of dyrk1a mRNA in neurons and its potential co-localization with the Mena protein was examined with immunofluorescence (IF) for Mena along with Fluorescent In Situ Hybridization (FISH) for dyrk1a mRNA, on cultured cortical neurons (FIG. 4A: a-d). A human dyrk1a probe that does not recognize the mouse mRNA was used as a negative control (FIG. 4A: a′-d′). Our results revealed extensive co-localization of the fluorescent signals along the axons and growth cones of neurons (FIG. 2A: i & ii white arrows). Line scans and correlation-coefficient analyses (Bolte and Cordelieres, 2006) indicated that the distributions of Mena protein and dyrk1a mRNA were significantly correlated across growth cone filopodia (FIGS. 4B i & ii and 4C). On the contrary, IF for Mena and Dyrk1a proteins did not reveal significant overlap of the fluorescent signals (FIG. 5A).

dyrk1a mRNA can be Co-recruited to the Mitochondrial Surface Along with Mena in a Re-localization Assay. To test whether Mena can affect the cytoplasmic localization of dyrk1a mRNA, a well-established mitochondrial sequestration assay (Bear et al., 2000) was utilized, in which the expression of a construct with the high-affinity EVH1 domain-binding motif DFPPPPXDE fused to a mitochondrial targeting sequence (“FP4-mito”), re-localizes endogenous Ena/VASP proteins to the mitochondrial surface (FIG. 5B). Expression of a control construct in which the EVH1-binding moiety is mutated to reduce affinity for EVH1 domains (“AP4-mito”) has only minimal effects on Ena/VASP relocalization. Using this assay, the vast majority of Mena (and its paralogs) is re-localized to the mitochondria, and in some cases, co-recruiting robustly interacting proteins (Gupton et al., 2012). Thus, it was reasoned that any mRNA associated with a Mena-containing RNP complex might also be co-recruited with Mena to the mitochondria by FP4-mito. Using nucleofection, the FP4- and AP4-mito constructs were expressed in primary neurons from E15.5 mouse brains. Fourty-eight hours after nucleofection and plating, FISH was performed to detect the mRNA of dyrk1a in FP4- and AP4-mito-transfected neurons. Expression of the constructs did not affect the total levels of dyrk1a mRNA (FIG. 5C). Interestingly, it was find that upon Mena translocation to the mitochondria, a significant amount of dyrk1a mRNA was also co-sequestered (FIGS. 4D: a-d & 4Ei), whereas in AP4-expressing control neurons dyrk1a mRNA localization remains unaffected (FIGS. 4D: a′-d′ and 2Eii). The correlation between Mena and dyrk1a mRNA was significantly elevated on the mitochondrial surface of FP4- vs AP4-mito transfected neurons (FIG. 4F). In contrast to the mRNA, IF analysis indicated that localization of the Dyrk1a protein was unaffected by FP4-Mito expression (FIG. 5D), consistent with the finding that there is no significant co-localization of Mena with the Dyrk1a proteins (FIG. 5A).

Taken together, the data demonstrates that Mena and dyrk1a mRNA interact specifically and co-localize within neuronal axons and growth cones, and that this interaction is robust enough to relocate the dyrk1a mRNA to the mitochondria in an Ena/VASP-dependent manner.

Mena is Necessary and Sufficient to Re-localize dyrk1a mRNA to the Mitochondria. To test whether other members of the Ena/VASP family can also be found in complex with mRNAs, VASP CLIP assays were performed. RT-PCR failed to detect Mena-associated mRNAs in complex with VASP (FIG. 5E). The inventors also failed to detect VASP associated with cytosolic mRNAs after Oligo(dT) Pulldown assays (data not shown), suggesting that the ability to associate with RNP complexes may be Mena-specific rather than a general property of Ena/VASP proteins. To test this hypothesis, FP4-mito was introduced into neurons isolated from Mena+/−; VASP+/+; EVL+/+ and from Mena−/−; VASP+/+; EVL+/+ E15.5 brains and analyzed the resulting effects on dyrk1a mRNA distribution. In the absence of Mena, both VASP (FIG. 5F) and EVL (not shown) were recruited to the mitochondria by FP4-mito, as expected, but the mRNA of dyrk1a was not (5G). Therefore Mena, unlike the other members of the Ena/VASP family, is necessary and sufficient to re-localize dyrk1a mRNA to the mitochondria, upon FP4-mito expression. As these data indicate that the ability to associate with dyrk1a and other mRNAs was specific to Mena, the inventors focused exclusively on characterizing the Mena-mRNA association in this study.

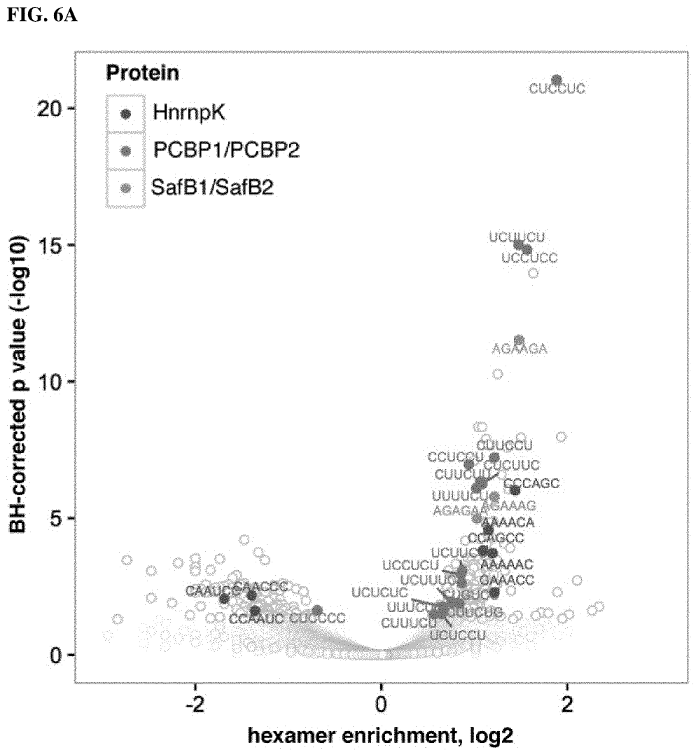

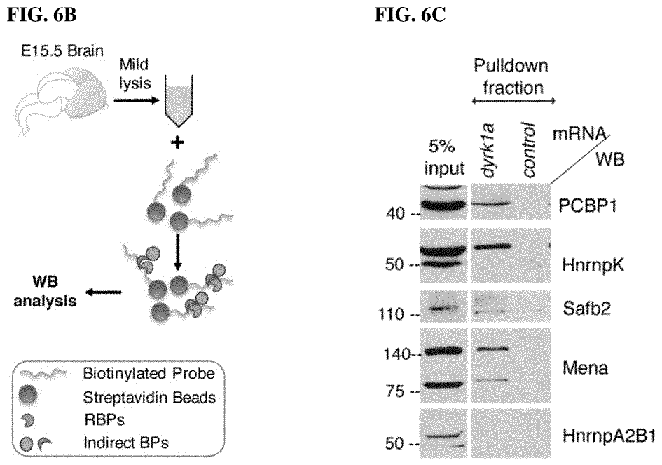

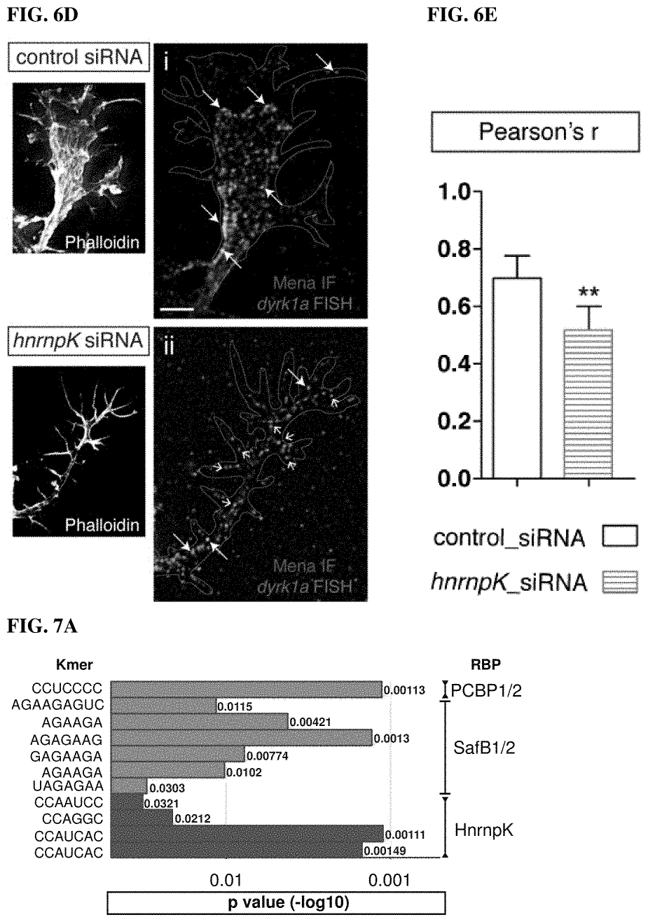

Candidate RBPs Mediating the Interaction Between Mena and mRNAs. To understand the biological significance of the association between Mena and specific mRNAs, how RBPs mediate this indirect interaction was investigated. A custom script was used to perform an unbiased analysis to identify sequences enriched within the 3′UTRs of the Mena-associated mRNAs that could serve as potential RBP binding motifs. To simplify the analysis, the inventors searched only for hexamer sequences, as they have been previously identified as highly efficient kmer motifs with minimal contextual binding effects (i.e. secondary structure formation, etc) (Lambert et al., 2014). Most of the enriched hexamers within the pool were found to correspond to binding motifs of RBPs that interact with Mena according to both our mass spectrometry data and co-IP validation experiments (FIG. 6A). More specifically the 3′UTR sequences derived from our Mena-HITS-CLIP data were enriched significantly for binding motifs of HnrnpK, PCBP1 (HnrnpE1), and Safb2, all of which were verified as interactors of Mena in brain lysates (FIGS. 3A & 2A).

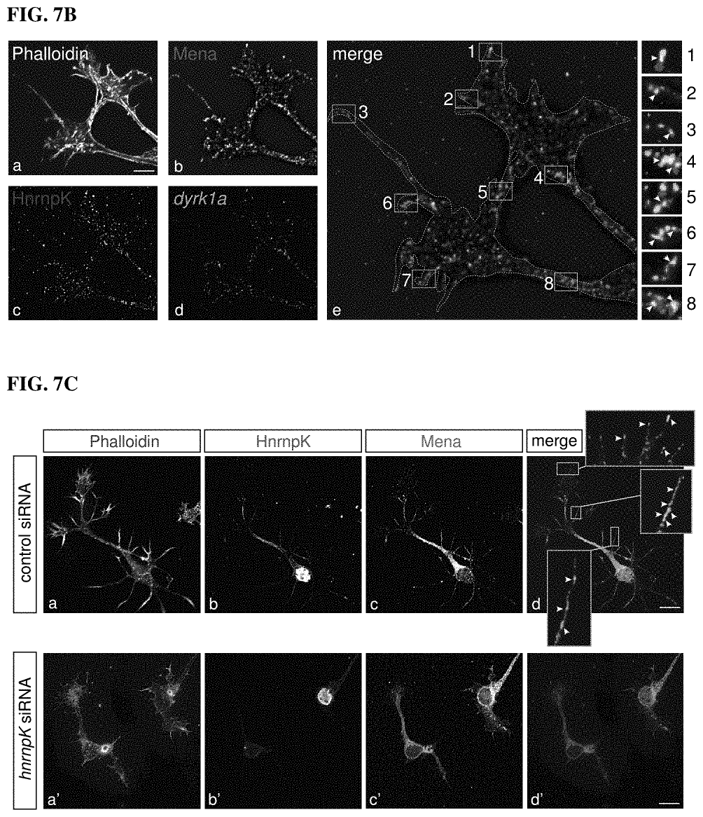

To test whether HnrnpK, SafB2 and PCBP1 could potentially mediate the indirect association of Mena with cytosolic mRNAs, whether they bind to the 3′UTR of the dyrk1a mRNA was examined. The sequences corresponding to the 3′UTR of dyrk1a in the Mena-HITS-CLIP data were analyzed using the RBPmap platform (rbpmap.technion.ac.il) (Akerman et al., 2009; Paz et al., 2010), and in good agreement with the hypothesis, putative binding sites for HnrnpK, PCBP1 and Safb2, among others, were found (FIG. 7A and Table 4, below). To confirm that PCBP1, HnrnpK, Safb2 and Mena could associate with the 3′UTR of dyrk1a mRNA, mRNA pulldown assays were performed using a biotinylated probe against the 3′UTR of dyrk1a mRNA to capture interacting protein complexes (FIG. 6B). Western blot analysis of the captured fraction from E15.5 brain lysates, revealed that all of three candidate RBPs, as well as Mena, were enriched in the bound fraction associated with the 3′UTR of dyrk1a, but not with a control biotinylated RNA probe, consistent with the previous in silico predictions (FIG. 6C). Further analysis using simultaneous dyrk1a FISH and IF for Mena and HnrnpK, revealed co-localization of the three fluorescent signals in the growth cones of primary neurons in culture (FIG. 7B), further consistent with the existence of a Mena-RNP complex.

TABLE 4
In silico-predicted binding sites for PCBP1,
Safb2 and HnrnpK on the 3′ UTR of dyrk1a.

| | Genomic | | | |
|---|---|---|---|---|
| RBP | Coordinate | Motif | K-mer | P-value |
| HnrnpK | chr16:94913334 | ccawmcc | ccaucac | 0.00149 |
| | chr16:94913352 | ccawmcc | ccaucac | 0.00111 |
| | chr16:94913399 | ccawmcc | ccaggcc | 0.0212 |
| | chr16:94913633 | ccawmcc | ccaaucc | 0.0321 |
| Safb1/2 | chr16:94913748 | hggagwa | uagagaa | 0.0303 |
| | chr16:94913751 | araaga | agaaga | 0.0102 |
| | chr16:94913750 | radacka | gagaaga | 0.00774 |
| | chr16:94913749 | agagavm | agagaag | 0.0013 |
| | chr16:94913751 | ggagwd | agaaga | 0.00421 |
| | chr16:94913751 | acgagagay | agaagaguc | 0.0115 |
| Pcbp1/2 | chr16:94914041 | ccyycch | ccucccc | 0.00113 |

Taken together, the data indicate that the Mena-interacting RBPs HnrnpK, PCBP1, and Safb2 could mediate the indirect association of Mena with dyrk1a, since all three can bind to the dyrk1a 3′UTR. Next, whether any of the candidate RBPs was responsible for the association of Mena with dyrk1a mRNA in neurons was examiner. To address this, the effect of depleting the RBPs on the extent of overlap between the Mena IF and dyrk1a FISH signals was analyzed. siRNA pools were introduced for each RBP that were introduced into primary neurons by co-nucleofection with a GFP plasmid to identify transfected neurons. As the nucleofection efficiency siRNAs into primary neurons is low, and the resulting protein depletion is limited by the non-proliferative phenotype of primary neurons, the efficacy of depletion by each siRNA pool was assessed using IF for the proteins. Using this approach, the inventors were able to generate convincing knockdown of HnrnpK, but not of either PCBP1 or Safb2 (FIG. 7C and data not shown). Whether co-localization between Mena and dyrk1a was sensitive to reduction in HnrnpK levels was then examined (FIG. 6D). HnrnpK depletion significantly reduced the signal overlap between Mena IF and dyrk1a FISH (FIG. 6D large white arrows), with more dyrk1a puncta lacking co-localized Mena signal (FIG. 6D small white arrows). This result is consistent with our hypothesis that HnrnpK is involved in mediating the interaction between Mena protein and dyrk1a mRNA.

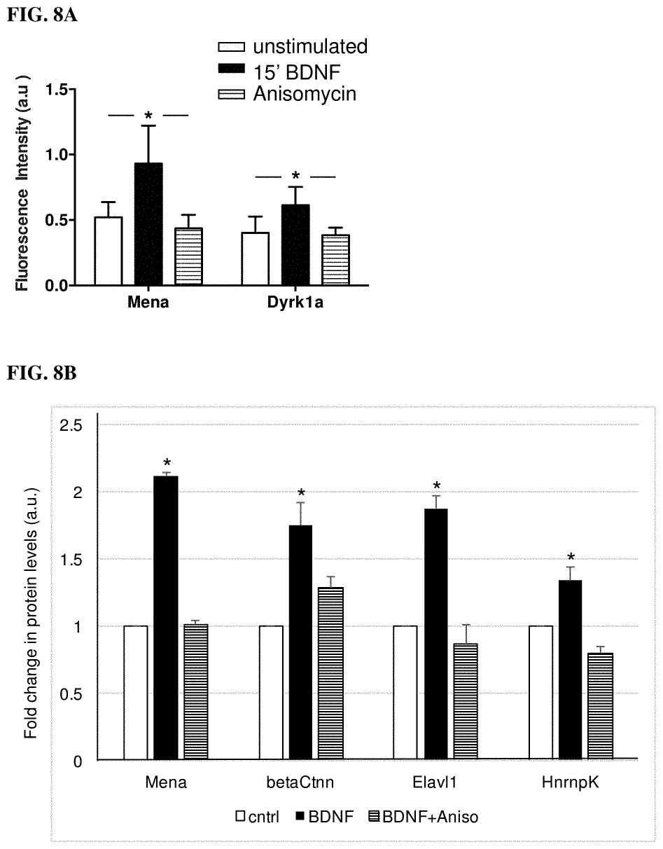

dyrk1a mRNA is Locally Translated in Axons upon Stimulation with BDNF. Based on the known roles of HnrnpK and PCBP1 in regulating cytosolic mRNA localization and translation (Thiele et al., 2016; Torvund-jensen et al., 2014), it was hypothesized that Mena-RNP complexes could facilitate transportation and/or local translation of the associated mRNAs, in axons and growth cones during development. To test this hypothesis, neurons were stimulated in culture with Brain Derived Neurotrophic Factor (BDNF) to elicit local translation (Jung et al., 2012; Santos et al., 2010; Schratt et al., 2004), and analyzed the abundance of Dyrk1a protein with and without stimulation. The protein levels of Mena was also tested as our sequencing data indicated the mena mRNA associated with the protein. IF on primary cortical neurons from E15.5 brains that were cultured for 48 hours, starved for 4 hours and then stimulated with BDNF for 15 minutes after, showed that both Mena and Dyrk1a fluorescence intensity levels significantly increased in the growth cones and axons of stimulated vs. unstimulated cells (FIG. 8A). This effect was blocked by the addition of the translation inhibitor anisomycin, indicating that the increase in the Dyrk1a and Mena IF signal resulted from BDNF-elicited protein synthesis (FIG. 8A).

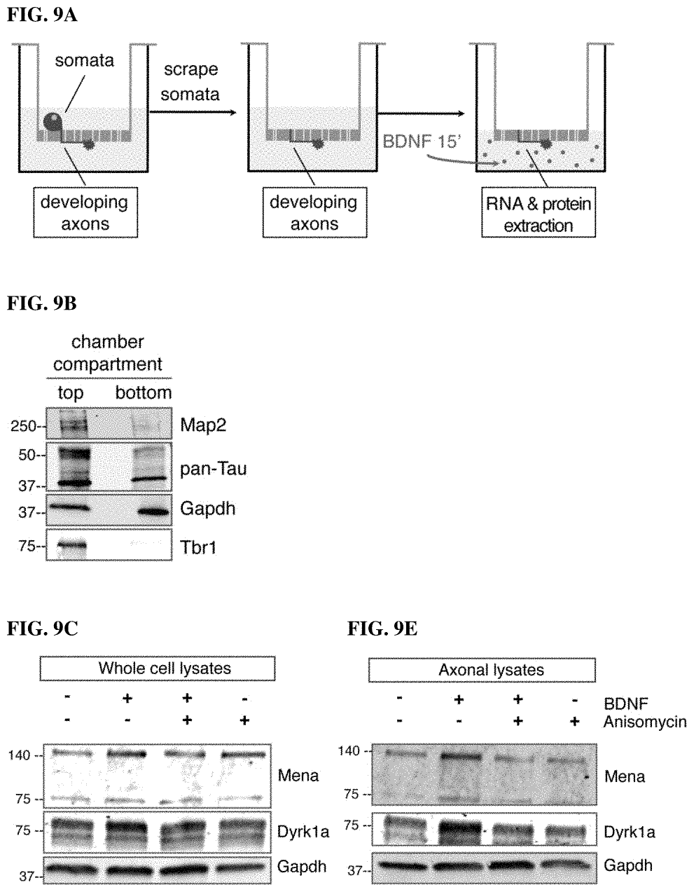

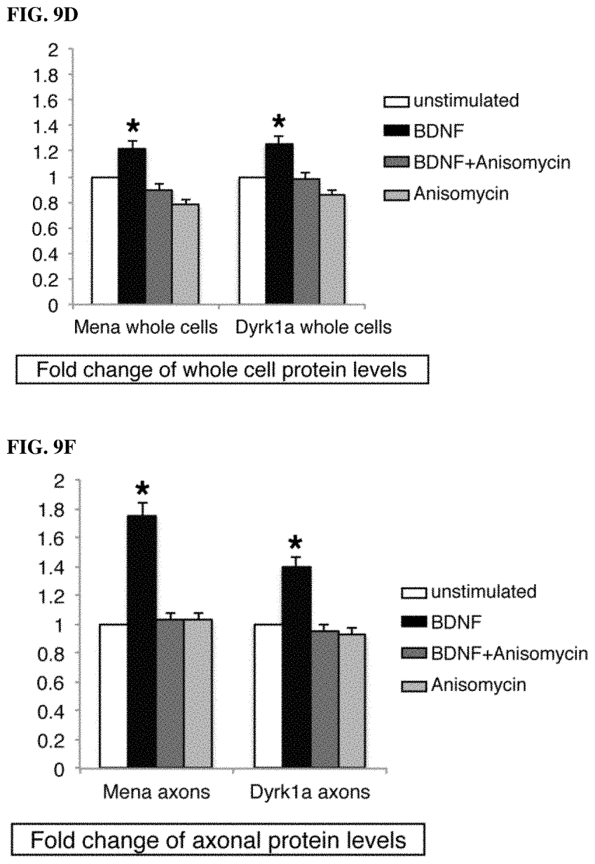

The BDNF-induced increase in axonal Mena and Dyrk1a proteins could arise from a global effect on their synthesis followed by protein trafficking into axons and growth cones, or, potentially, from local translation of axon-localized mRNAs. To investigate this possibility, cortical neurons were cultured on the top compartment of transwell chambers separated by filters with 1 μm membrane pores (FIG. 9A) that allow neuronal processes, but not neuronal cell bodies, to extend onto the bottom of the filter, and permit their physical fractionation from the soma. Thirty-six to fourty-eight hours after plating primary neurons on top of the filter, material harvested from the top and bottom compartments of the chamber was isolated and used to prepare lysates. Western blot analysis of lysates from the top and bottom compartments with known axon (pan-Tau), dendrite (Map2) and nuclear (Tbr1) markers, 36-48 hours after plating, verified that this assay could successfully separate somata from neuronal processes (FIG. 9B: absence of Tbr1 at the bottom compartment), and that the neuronal processes isolated from the bottom were primarily axons as opposed to dendrites (FIG. 9B: enrichment of Tau and barely detectable levels of Map2), as anticipated based on the short time-window of the cultures. To assess protein synthesis using this system, neurons were allowed to grow for 36 hours followed by starvation for 4 hours to minimize transcriptional and translational activity. Following starvation, the cell bodies were gently scraped off and removed from the top compartment of the filters, while the retained severed axons were stimulated with BDNF for 15 minutes along with controls in which the entire unscraped filter was stimulated, to measure local and global protein synthesis, respectively. To ensure de novo translational events were monitored, controls in which anisomycin was used to block protein synthesis were performed. Western blot analysis revealed a significant increase in the of Mena and Dyrk1a protein levels upon BDNF stimulation, compared to untreated and anisomycin-treated neurons, both globally, in whole cells, and even more evidently, locally in isolated axons that were stimulated (FIGS. 9C-D and 9E-F respectively). BDNF-elicited local translation of additional Mena-associated mRNAs was also observed using this assay (FIG. 8B).

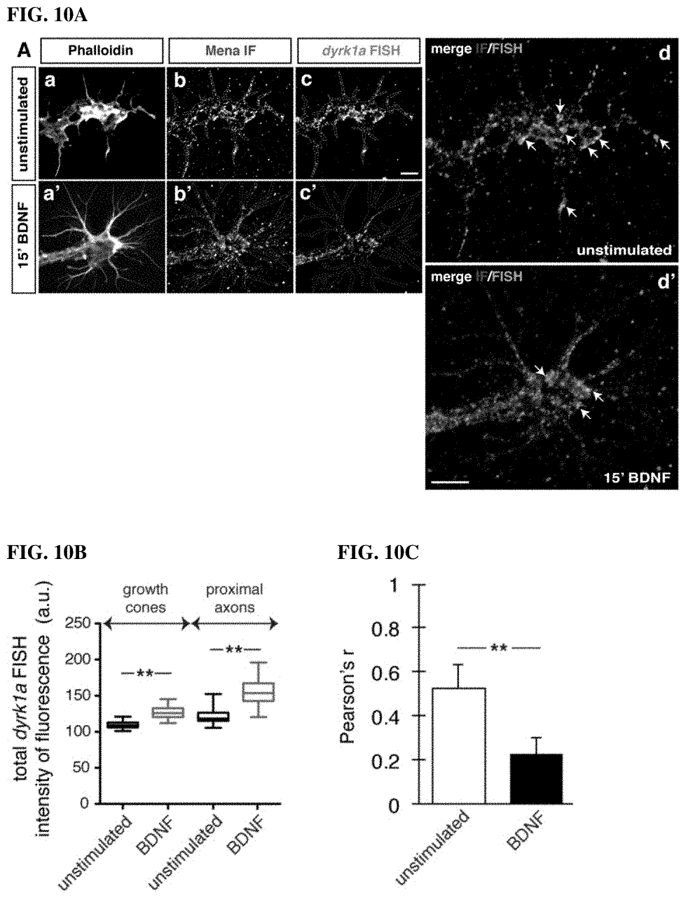

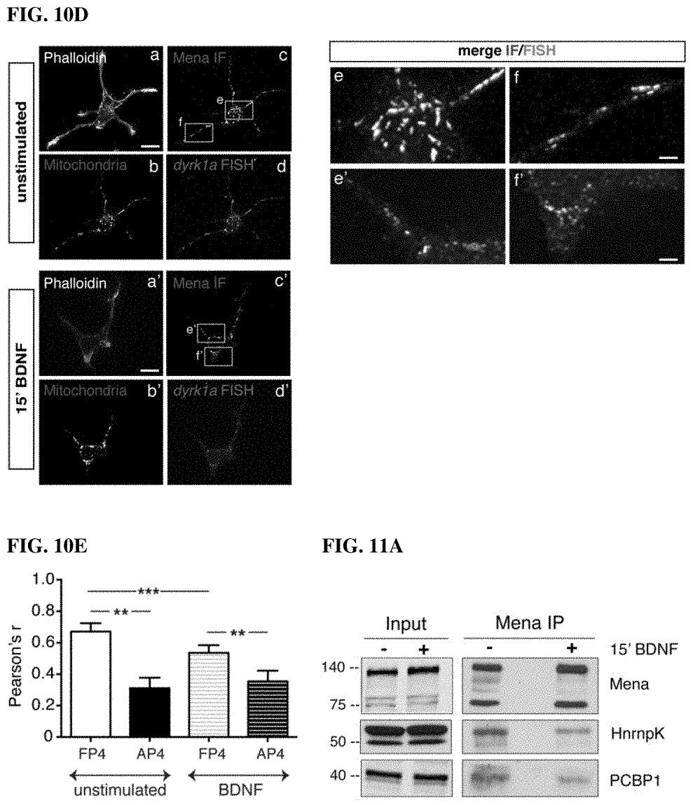

BDNF Stimulation Decreases the Association between Mena and dyrk1a mRNA. Given that BDNF can induce translation of dyrk1a mRNA in developing neurons, whether and how the stimulation affects the association of Mena with dyrk1a was examined. First, whether the extent of Mena protein co-localization with dyrk1a mRNA in growth cones was altered upon stimulation was examined. IF for Mena and FISH for dyrk1a in cortical neurons was performed with and without BDNF stimulation (FIG. 10A). Notably, an increase in the FISH signal of dyrk1a mRNA after BDNF stimulation was observed, both in the growth cones and their proximal axon part (FIG. 10B), indicating that increases in dyrk1a transcription, mRNA transport, or both occur upon BDNF stimulation. Interestingly, the overlap between the Mena IF and dyrk1a FISH signals was significantly decreased by BDNF treatment, raising the possibility that Mena:dyrk1a-containing complexes may dissociate upon stimulation (FIG. 10C). To test this hypothesis, the mitochondria re-localization assay described above (FIG. 10B) was utilized. It was observed that the amount of dyrk1a co-recruited with Mena to mitochondria in FP4-mito expressing neurons was reduced significantly by BDNF treatment (FIG. 10D) compared to unstimulated neurons (FIG. 10E). This result is in agreement with our hypothesis that BDNF stimulation decreases the association between Mena and dyrk1a mRNA.

Overall, the results demonstrate that, while BDNF stimulation increases total dyrk1a mRNA levels and local translation in axons, it reduces the association of Mena and dyrk1a.

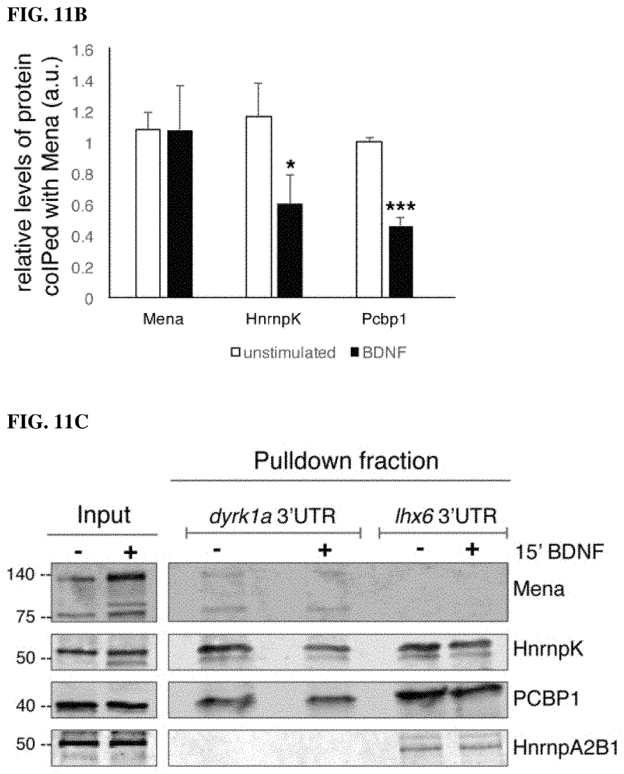

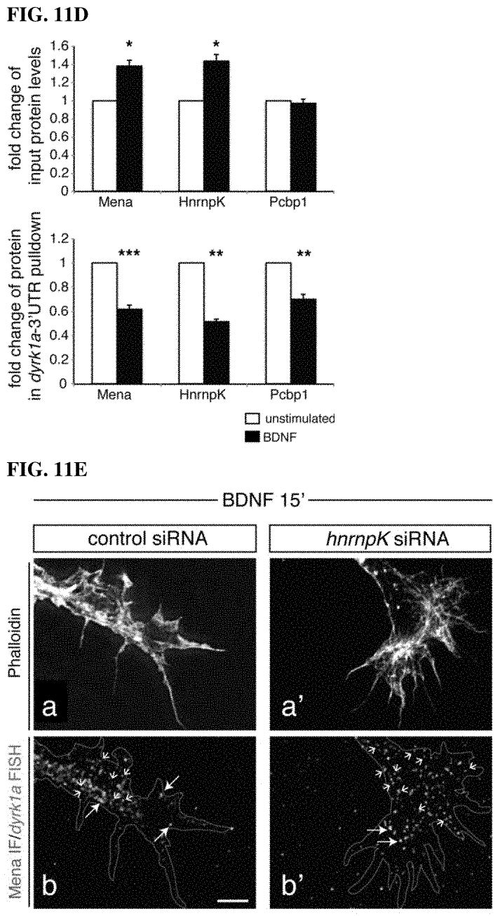

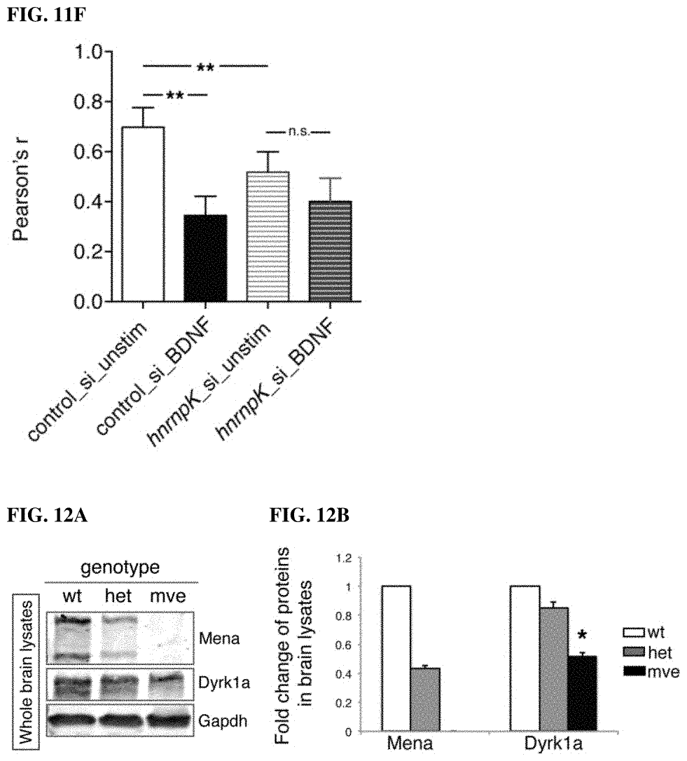

BDNF Stimulation Results in Partial Dissociation of the Mena;dyrk1a RNP Complexes. We next investigated the effects of BDNF stimulation on Mena:RNP complexes. We performed coIP experiments and found that the levels of HnrnpK and PCBP1 recovered with Mena were significantly reduced in lysates of BDNF vs. unstimulated cultured primary neurons (FIGS. 11A and 11B). Taken together, the results suggest that BDNF stimulation induces Mena-RBP complex dissociation, which could lead to dissociation dyrk1a mRNA from with Mena. To test this hypothesis, pulldowns were performed using biotinylated dyrk1a 3′UTR from the lysates of neurons with or without BDNF stimulation. An irrelevant 3′UTR from lhx6, an mRNA not detected in the Mena-CLIPseq data (Table 3) was used as a negative control for the binding assay. In good agreement with previous findings, BDNF induced a significant increase in the protein levels of Mena and HnrnpK (FIGS. 11C input and 11D and FIG. 8B), however, the amounts of Mena, HnrnpK and Pcbp1 pulled-down with the 3′UTR of dyrk1a mRNA were significantly decreased by BDNF stimulation (FIGS. 11C pulldown fraction and 11D). Together, these data indicate that, in addition to eliciting translation of dyrk1a, BDNF stimulation triggers dissociation of Mena from its interacting RBPs and from the dyrk1a mRNA.

Based on the results, it was hypothesized that HnrnpK, which contributes to the association between Mena and dyrk1a, would be required to detect the BDNF-elicited reduction in their co-localization. FISH for dyrk1a and IF for Mena was performed in HnrnpK-depleted neurons (FIG. 11E) and observed that the overlap between the fluorescence was significantly reduced compared to controls in the absence of HnrnpK in unstimulated cells (FIGS. 6D and 11F). Interestingly, HnrnpK-depleted neurons failed to exhibit any further significant reduction of Mena and dyrk1a co-localization after BDNF stimulation (FIG. 11F)

Taken together, the results indicate that the association of dyrk1a mRNA with Mena depends, at least in part, on the presence of HnrnpK in unstimulated cells, suggesting that BDNF-elicited decreases in Mena:HnrnpK could contribute to Mena dissociation from dyrk1a-containing RNPs.

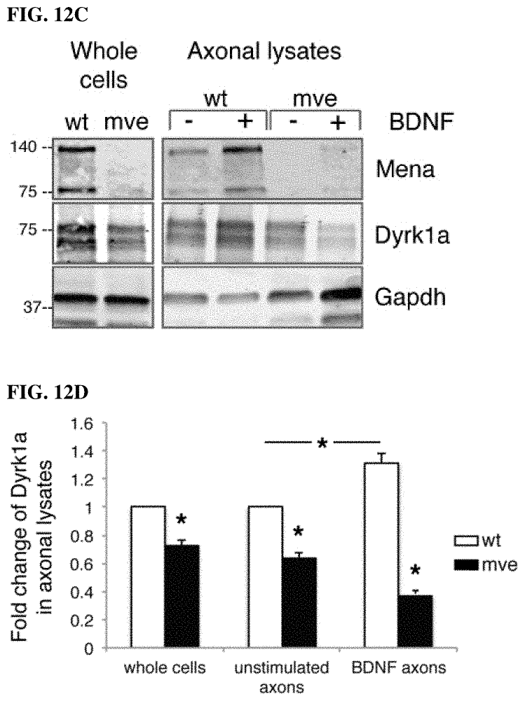

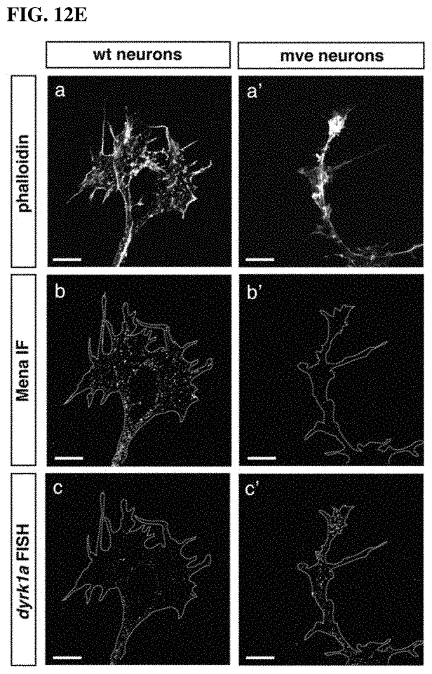

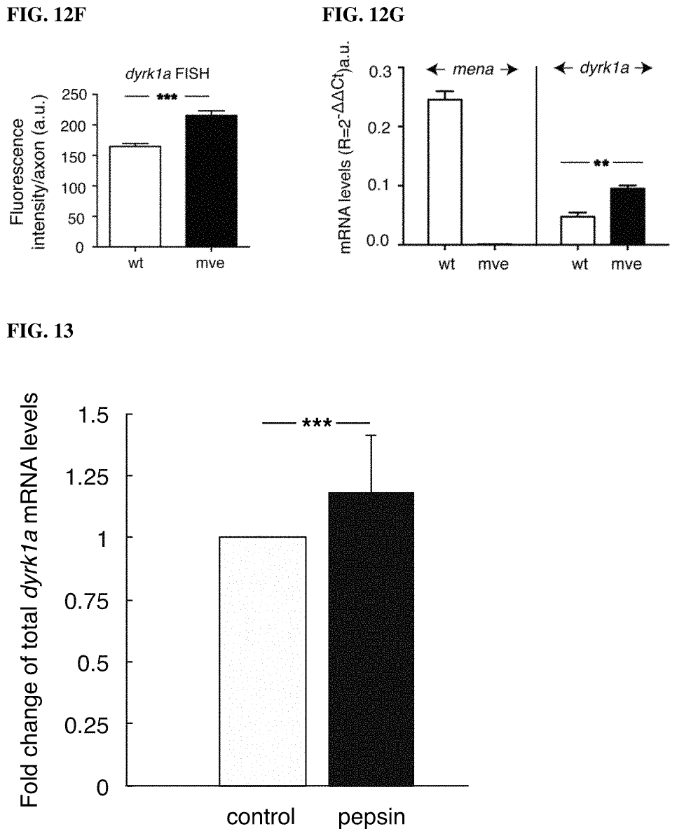

The absence of Mena disrupts Dyrk1a translation, but does not affect axonal targeting of the dyrk1a mRNA. Thus far, the data reveals an association between Mena and dyrk1a, through the formation of Mena-RNP complexes, and a potential role for those complexes in dyrk1a mRNA translation. The requirement for Mena in dyrk1a localization and translation was investigated using material from Mena-deficient animals. Western blot analysis of E15.5 whole brain lysates from Mena wt (wt: Mena+/+; VASP−/−; EVL−/−), Mena heterozygous (het: Mena+/−; VASP−/−; EVL−/−) and Mena-deficient (mve: Mena−/−; VASP−/−; EVL−/−) embryos revealed a significant decrease in Dyrk1a protein levels in Mena-null brains (FIGS. 12A and 12B).

The results raise the possibility that translation of the dyrk1a mRNA requires Mena-containing complexes. To test this, the effect of BDNF stimulation on isolated axons, as described above (FIG. 9A), was examined using cortical neurons isolated wt and mve emryos by western blot. It was observed that mve axons had lower Dyrk1a protein levels compared to control, and that Dyrk1a levels failed to increase upon BDNF stimulation (FIGS. 12C and 12D).

To verify that the Dyrk1a protein level reduction observed in mve neurons arose from defective translation rather than abnormal mRNA transport, FISH for dyrk1a on mve neurons was performed. While dyrk1a mRNA was normally targeted to axons and growth cones in both samples, dyrk1a mRNA levels were significantly increased in mve neurons, compared to control cells (FIGS. 12E and 12F). This could be explained by compensatory transport from the soma, and/or mRNA unmasking in the absence of Mena. Signal increases arising from mRNA unmasking have been shown to occur upon neuronal activation, as mRNAs are released from protein complexes during translation or decay (Buxbaum et al., 2014). Pepsin treatment on wt neurons in culture as previously described (Buxbaum et al., 2014), followed by FISH, revealed a significant increase in the dyrk1a signal between untreated and pepsin-treated cells (FIG. 13), indicating that part of dyrk1a RNA is masked by proteins in the absence of stimulation. To analyze the abundance dyrk1a mRNA in mve neurons in a protein complex-independent manner, total mRNA was isolated and quantitative PCR analysis for dyrk1a was performed. Interestingly, significantly higher dyrk1a mRNA levels in mve, versus control neurons (FIG. 12G), was observed. The increased abundance of dyrk1a mRNA in the absence of Mena likely arises as a consequence of elevated dyrk1a transcription, increased mRNA stability, or both, potentially triggered by impaired translation of dyrk1a mRNA.

Altogether, our data highlight the importance of Mena for translation of the dyrk1a mRNA in developing neurons, and indicate a novel role for Mena in the regulation of local protein synthesis of Dyrk1a, and potentially more of the mRNAs that associate with it in axons.

Discussion

An unanticipated role was found for Mena, but not for its paralogs VASP and EVL, as a key regulator of dyrk1a mRNA translation in neurons, and a set of Mena-associated mRNAs have been identified that may be similarly regulated. Using BDNF to elicit protein synthesis (Santos et al., 2010; Schratt et al., 2004), it was demonstrated that the mRNAs encoding Mena and Dyrk1a can be locally translated in axons severed from their cell bodies, and that this de novo protein synthesis is Mena-dependent. These findings raise the intriguing possibility that Mena could act as a regulatory node that coordinates and balances actin polymerization and local protein synthesis in response to specific cues during neuronal development and, potentially, in adult neuroplasticity.

Interestingly, similar dual roles have been reported for CYFIPs, which can function either as a regulator of Arp2/3-mediated actin nucleation through the WAVE-complex, or as a local translation inhibitor in synaptic spines, via direct binding to the FMR1 RBP (De Rubeis et al., 2013), and for APC, which regulates microtubule dynamics, mRNA enrichment in filopodia (Mili et al., 2008), and axonal localization and translation of β2B-tubulin mRNA (Preitner et al., 2014). Notably, the mRNA set identified as associated with Mena, is significantly different from mRNAs already known to be locally translated and associated with well-described RBPs, including FMR1, APC, Staufen, and Barentsz (Ascano et al., 2012; Balasanyan and Arnold, 2014; Brown et al., 2001; Fritzsche et al., 2013; Preitner et al., 2014), minus few exceptions (i.e. β-catenin (Baleriola and Hengst, 2014; Deglincerti and Jaffrey, 2012), suggesting that the Mena-containing complexes represent a novel RNP complex involved in localized mRNA translation in axons.

Mena associates indirectly with dyrk1a and other cytosolic mRNAs in an RNP containing the RBPs HnrnpK, PCBP1 and Safb2. Binding motifs for these three RBPs were enriched significantly in Mena-complex mRNAs, and they were all detected in pulldown assays with the dyrk1a 3′UTR. HnrnpK plays a critical role in linking Mena to mRNAs as HnrnpK depletion significantly reduced association between Mena and dyrk1a mRNA. Exactly how the Mena-RNP complex forms and connects Mena to specific mRNAs will require further investigation, though it is noteworthy that HnrnpK and Safb2 both contain LP4 motifs, which can mediate direct binding to the EVH1 domain of Ena/VASP proteins (Niebuhr et al., 1997), and that Safb2 also contains a region of similarity to the LERER domain in Mena (Townson et al., 2003).

Two of the RBPs found here to associate with Mena, HnrnpK and PCBP1, have varied roles in RNA metabolism, including regulation of mRNA translation (Gebauer and Hentze, 2004; Ostareck-lederer et al., 2002; Thiele et al., 2016; Torvund-jensen et al., 2014). Interestingly, HnrnpK and PCBP1 can form complexes that inhibit translation initiation when bound to the 3′UTRs of target mRNAs (Gebauer and Hentze, 2004). But how can Mena be associated with an mRNA and positively regulate its translation, when present in a complex that silences dyrk1a translation? The results here are consistent with the possibility that dyrk1a is translationally silenced by the HnrnpK and PCBP1 moieties in the Mena-RNP complex, and that de-repression of dyrk1a translation requires Mena. In Mena-deficient neurons, steady state levels of Dyrk1a protein are reduced, and BDNF stimulation fails to induce dyrk1a translation. The data shows that BDNF stimulation disrupts Mena's association with HnrnpK and PCBP1, as well as the recovery of Mena, HnrnpK and PCBP1 in pulldowns using the 3′UTR of dyrk1a, supports a speculative model in which dissociation of the Mena-RNP complex releases dyrk1a mRNA from its translationally-inhibited state.

The Mena-RNP complex is significantly enriched for many mRNAs encoding proteins involved in NS development and function, including dyrk1a. Dyrk1a is a dosage-sensitive, dual-specificity protein kinase that fulfills key roles during development and in tissue homeostasis, and its dysregulation results in multiple human pathologies (Chen et al., 2013; Hammerle et al., 2003; O'Roak et al., 2012; Qian et al., 2013; Tejedor and Hammerle, 2011). It is present in both the nucleus and cytoplasm of mammalian cells, although its nuclear function remains unclear (Di Vona et al., 2015; Tejedor and Hammerle, 2011). Human Dyrk1a maps to chromosome 21, and it is overexpressed in Down syndrome (DS) individuals and DS mouse models. This alteration has been correlated with a wide range of the pathological phenotypes associated to DS, such as motor alterations, retinal abnormalities, osteoporotic bone phenotype, craniofacial dysmorphology, or increased risk of childhood leukemia (Arron et al., 2006; Kim et al., 2016; Malinge et al., 2012; Ortiz-Abalia et al., 2008; van Bon et al., 2015). In addition, a few cases of truncating mutations in one Dyrk1a allele have been described in patients with general growth retardation and severe primary microcephaly (Van Bon et al., 2011), highlighting the extreme dosage sensitivity of this gene. Moreover, and as an indication of the pleiotropic activities of Dyrk1a, dysregulation of this kinase has also been linked to tumor growth and pancreatic dysfunction (Fernandez-Martinez et al., 2015; Rachdi et al., 2014).

Like most of the mRNAs identified in this study, dyrk1a contains multiple binding sites for the Mena-complex in its 3′UTR, consistent with the data herein demonstrating that the Mena-RNP complex regulates local synthesis of Dyrk1a protein. Given the extreme dosage sensitivity of Dyrk1a and its implication in numerous neurodevelopmental disorders, these findings that Dyrk1a protein levels are regulated in a Mena-dependent manner in axons raises the intriguing possibility that dysregulation of the Mena-RNP complex may contribute to such disorders. Additional mRNAs that are associated with Mena, like the validated targets β-catenin and elav11 (HuR) are also implicated in multiple developmental processes and pathophysiological conditions (Alami et al., 2014; Blanco et al., 2016; Holland et al., 2013; Krumm et al., 2014; Li et al., 2017; Lu et al., 2014; O'Roak et al., 2012; Wang et al., 2016). Therefore, the Mena-RNP complex may represent a target for the development of novel therapeutic strategies for multiple disease pathologies.

Interestingly, the Mena-RNP complex contains mena mRNA, which harbors multiple binding sites for the complex in its 3′UTR, raising the possibility that Mena regulates translation of its own mRNA. Mena-regulated translation of β-catenin could also affect mena mRNA abundance since β-catenin can regulate mena transcription (Najafov et al., 2012). These findings are consistent with the potential existence of regulatory feedback loops that control Mena protein abundance at the transcriptional and translational levels.

Neurons deficient for Ena/VASP proteins fail to respond properly to Netrin and Slit (Bashaw et al., 2000; Dent et al., 2011; Dent and Gertler, 2003; Kwiatkowski et al., 2007; Lebrand et al., 2004; Mcconnell et al., 2016), two axon guidance cues that require local translation (Campbell et al., 2001; Jung et al., 2012; Jung and Holt, 2011). The results here raise the interesting possibility that, in addition to its established role in regulating filopodia dynamics in response to Netrin and Slit, Mena could contribute to local translation-dependent responses to these cues. Interestingly, both Mena and HnrnpK have been implicated in synapse formation and plasticity (Folci et al., 2014; Giesemann et al., 2003; Li et al., 2005; Lin et al., 2007; Proepper et al., 2011), raising the possibility that their synaptic functions involve regulated translation by the Mena-RNP complex.

Summary of Sequence Listing

The specification includes a Sequence Listing appended herewith, which includes sequences, as follows:

SEQ ID NO: 1 - mouse GAPDH
5′-catgttccagtatgactccactc
SEQ ID NO: 2 - mouse GAPDH
3′-ggcctcaccccatttgatgt
SEQ ID NO: 3 - mouse Mena
5′-gggcagaaagattcaagacc
SEQ ID NO: 4 - mouse Mena
3′-gcgaagacattggcatcc
SEQ ID NO: 5 - mouse Dyrk1a
5′-caaacggagtgcaatcaaga
SEQ ID NO: 6 - mouse Dyrk1a
3′-agcacctctggagaccgata
SEQ ID NO: 7 - mouse Robo1
5′-catcaagaggatcagggagc
SEQ ID NO: 8 - mouse Robo1
3′-ggttgtcttcagctttcagtttc
SEQ ID NO: 9 - mouse Elavl1
5′-agccaatcccaaccagaac
SEQ ID NO: 10 - mouse Elavl1
3′-acaccagaaatcccactcatg
SEQ ID NO: 11 - mouse β-Ctnn
5′-ctatcccagaggctttatccaag
SEQ ID NO: 12 - mouse β-Ctnn
3′-ccagagtgaaaagaacggtagc
SEQ ID NO: 13 - mouse Khsrp
5′-gccaatcagactacaccaagg
SEQ ID NO: 14 - mouse Khsrp
3′-gccacttgtgttgcttcttg
SEQ ID NO: 15 - mouse Eif4ebp2
5′-ccatctgcccaatatccctg
SEQ ID NO: 16 - mouse Eif4ebp2
3′-tgtccatctcaaactgagcc
SEQ ID NO: 17 - mouse Vamp2
5′-aagttgtcggagctggatg
SEQ ID NO: 18 - mouse Vamp2
3′-cgcagatcactcccaagatg
SEQ ID NO: 19 - Mouse Mena/ENAH
(>sp|Q03173|ENAH_MOUSE Protein enabled
homolog OS = Mus musculus GN = Enah PE = 1
SV = 2)
SEQ ID NO: 20 - Human Mena/ENAH
(>sp|Q8N8S7|ENAH_HUMAN Protein enabled
homolog OS = Homo sapiens GN = ENAH PE = 1
SV = 2)
SEQ ID NO: 21 - Mouse DYR1A
(>sp|Q61214|DYR1A_MOUSE Dual specificity
tyrosine-phosphorylation-regulated kinase 1A
OS = Mus musculus GN = Dyrk1a PE = 1 SV = 1)
SEQ ID NO: 22 - Human DYR1A
(>sp|Q13627|DYR1A_HUMAN Dual specificity
tyrosine-phosphorylation-regulated kinase 1A
OS = Homo sapiens GN = DYRK1A PE = 1 SV = 2)
SEQ ID NO: 23 - Mouse Hnrpk
(>sp|P61979|HNRPK_MOUSE Heterogeneous nuclear
ribonucleoprotein K OS = Mus musculus
GN = Hnrnpk PE = 1 SV = 1)
SEQ ID NO: 24 - Human Hnrpk
(>sp|P61978|HNRPK_HUMAN Heterogeneous nuclear
ribonucleoprotein K OS = Homo sapiens
GN = HNRNPK PE = 1 SV = 1)
SEQ ID NO: 25 - Mouse PCBP1
(>sp|P60335|PCBP1_MOUSE Poly(rC)-binding
protein 1 OS = Mus musculus GN = Pcbp1
PE = 1 SV = 1)
SEQ ID NO: 26 - Human PCBP1
(>sp|Q15365|PCBP1_HUMAN Poly(rC)-binding
protein 1 OS = Homo sapiens GN = PCBP1
PE = 1 SV = 2)
SEQ ID NO: 27 - Mouse Amyloid beta A4
(>sp|P12023|A4_MOUSE Amyloid beta A4 protein
OS = Mus musculus GN = App PE = 1 SV = 3)
SEQ ID NO: 28 - Human Amyloid beta A4
(>sp|P05067|A4_HUMAN Amyloid beta A4 protein
OS = Homo sapiens GN = APP PE = 1 SV = 3)
SEQ ID NO: 29 - Mouse SAFB2
(>sp|Q80YR5|SAFB2_MOUSE Scaffold attachment
factor B2 OS = Mus musculus GN = Safb2 PE = 1
SV = 2)
SEQ ID NO: 30 - Human SAFB2
(>sp|Q14151|SAFB2_HUMAN Scaffold attachment
factor B2 OS = Homo sapiens GN = SAFB2 PE = 1
SV = 1)
SEQ ID NO: 31 - Mouse CTNB1
(>sp|Q02248|CTNB1_MOUSE Catenin beta-1
OS = Mus musculus GN = Ctnnb1 PE = 1 SV = 1)
SEQ ID NO: 32 - Human CTNB1
(>sp|P35222|CTNB1_HUMAN Catenin beta-1
OS = Homo sapiens GN = CTNNB1 PE = 1 SV = 1)
SEQ ID NO: 33 - Mouse SHAN2
(>sp|Q80Z38|SHAN2_MOUSE SH3 and multiple
ankyrin repeat domains protein 2 OS = Mus
musculus GN = Shank2 PE = 1 SV = 2)
SEQ ID NO: 34 - Human SHAN2
(>sp|Q9UPX8|SHAN2_HUMAN SH3 and multiple
ankyrin repeat domains protein 2 OS = Homo
sapiens GN = SHANK2 PE = 1 SV = 3)
SEQ ID NO: 35 - Mouse PTEN
(>sp|O08586|PTEN_MOUSE Phosphatidylinositol
3,4,5-trisphosphate 3-phosphatase and dual-
specificity protein phosphatase PTEN
OS = Mus musculus GN = Pten PE = 1 SV = 1)
SEQ ID NO: 36 - Human PTEN
(>sp|P60484|PTEN_HUMAN Phosphatidylinositol
3,4,5-trisphosphate 3-phosphatase and dual-
specificity protein phosphatase PTEN
OS = Homo sapiens GN = PTEN PE = 1 SV = 1)
SEQ ID NO: 37 - Mouse VAMP2
(>sp|P63044|VAMP2_MOUSE Vesicle-associated
membrane protein 2 OS = Mus musculus
GN = Vamp2 PE = 1 SV = 2)
SEQ ID NO: 38 - Human VAMP2
(>sp|P63027|VAMP2_HUMAN Vesicle-associated
membrane protein 2 OS = Homo sapiens
GN = VAMP2 PE = 1 SV = 3)
SEQ ID NO: 39 - Mouse ELAV1
(>sp|P70372|ELAV1_MOUSE ELAV-like protein 1
OS = Mus musculus GN = Elavl1 PE = 1 SV = 2)
SEQ ID NO: 40 - Human ELAV1
(>sp|Q15717|ELAV1_HUMAN ELAV-like protein 1
OS = Homo sapiens GN = ELAVL1 PE = 1 SV = 2)
SEQ ID NO: 41 - Mouse ROB01
(>sp|O89026|ROBO1_MOUSE Roundabout homolog 1
OS = Mus musculus GN = Robo1 PE = 1 SV = 1)
SEQ ID NO: 42 - Human ROB01
(>sp|Q9Y6N7|ROBO1_HUMAN Roundabout homolog 1
OS = Homo sapiens GN = ROBO1 PE = 1 SV = 1)
SEQ ID NO: 43 - Mouse Mena/ENAH cDNA
(>ENA|AAC52866|AAC52866.1 Mus musculus
(house mouse) neural variant mena+++)
SEQ ID NO: 44 - Human Mena/ENAHcDNA
(>ENA|AAQ08487|AAQ08487.1 Homo sapiens
(human) mena protein)
SEQ ID NO: 45 - Mouse Dyrk1a-cDNA
(>ENA|AAC52994|AAC52994.2 Mus musculus
(house mouse) mp86)
SEQ ID NO: 46 - Human Dyrk1a cDNA
(>ENA|AAI56310|AAI56310.1 synthetic
construct partial dual-specificity tyrosine-
(Y)-phosphorylation regulated kinase 1A)
SEQ ID NO: 47 - Mouse HnrnpK cDNA
(>ENA|BAB27614|BAB27614.1 Mus musculus
(house mouse) hypothetical protein)
SEQ ID NO: 48 - Human HnrnpK cDNA
(>ENA|AAB20770|AAB20770.1 Homo sapiens
(human) heterogeneous nuclear
ribonucleoprotein complex K)
SEQ ID NO: 49 - Mouse Pcbp1 cDNA
(>ENA|AAD51920|AAD51920.1 Mus musculus
(house mouse) RNA-binding protein alpha-CP1)
SEQ ID NO: 50 - Human Pcbp1 cDNA
(>ENA|AAA91317|AAA91317.1 Homo sapiens
(human) alpha-CP1)
SEQ ID NO: 51 - Mouse APP cDNA
(>NM_001198823.1:150-2462 Mus musculus
amyloid beta (A4) precursor protein (App),
transcript variant 1, mRNA)
SEQ ID NO: 52 - Human APP cDNA
(>ENA|AAB59502|AAB59502.2 Homo sapiens
(human) amyloid-beta protein)
SEQ ID NO: 53 - Mouse Safb2 cDNA
(>NM_001029979.2:140-3115 Mus musculus
scaffold attachment factor B2 (Safb2), mRNA)
SEQ ID NO: 54 - Human Safb2 cDNA
(>ENA|AAC14666|AAC14666.1 Homo sapiens
(human) KIAA0138)
SEQ ID NO: 55 - Mouse ctnb1 cDNA
(>ENA|AAA37280|AAA37280.1 Mus musculus
(house mouse) beta-catenin)
SEQ ID NO: 56 - Human ctnb1 cDNA
(>ENA|AAH58926|AAH58926.1 Homo sapiens
(human) catenin (cadherin-associated
protein), beta 1, 88 kDa)
SEQ ID NO: 57 - Mouse Shank2 cDNA
(>XM_006508533.1:483-4913 PREDICTED: Mus
musculus SH3/ankyrin domain gene 2 (Shank2),
transcript variant X17, mRNA)
SEQ ID NO: 58 - Human Shank2 cDNA
(>ENA|BAH37017|BAH37017.1 Homo sapiens
(human) proline-rich synapse associated
protein 1)
SEQ ID NO: 59 - Mouse Pten cDNA
(>ENA|AAC53118|AAC53118.1 Mus musculus
(house mouse) MMAC1)
SEQ ID NO: 60 - Human Pten cDNA
(>ENA|AAB66902|AAB66902.1 Homo sapiens
(human) protein tyrosine phosphatase)
SEQ ID NO: 61 - Mouse Vamp2 cDNA
(>ENA|AAB03463|AAB03463.1 Mus musculus
(house mouse) VAMP-2)
SEQ ID NO: 62 - Human Vamp2 cDNA
(>ENA|AAH02737|AAH02737.2 Homo sapiens
(human) vesicle-associated membrane
protein 2 (synaptobrevin 2))
SEQ ID NO: 63 - Mouse Elavl1 cDNA
(>ENA|BAC37892|BAC37892.1 Mus musculus
(house mouse) hypothetical protein)
SEQ ID NO: 64 - Human Elavl1 cDNA
(>ENA|AAH03376|AAH03376.2 Homo sapiens
(human) ELAV (embryonic lethal, abnormal
vision, Drosophila)-like 1 (Hu antigen R))
SEQ ID NO: 65 - Mouse Robo1 cDNA
(>ENA|CAA76850|CAA76850.1 Mus musculus
(house mouse) Dutt1 protein)
SEQ ID NO: 66 - Human Robo1 cDNA
(>ENA|AAC39575|AAC39575.1 Homo sapiens
(human) roundabout 1)

### Specific Embodiments

In an aspect, the present disclosure provides a method of modulating protein expression from a Mena-ribonucleoprotein (RNP) complex, the method comprising: administering to a subject an agent that: (a) inhibits protein expression by inhibiting Mena translation, Mena transcription, or the association of Mena with the Mena-RNP complex, or (b) promotes protein expression by: (i) inhibiting the expression of at least one of HnmpK, PCBP1, or both, or (ii) dissociating at least one of HnmpK, PCBP1, or both, from (or preventing the association of at least one of HnmpK, PCBP1, or both with) the Mena-RNP complex in the cell.

In any aspect or embodiment of the present disclosure, the agent that inhibits protein expression is selected from an antisense agent, an RNAi agent, an antibody or an antigen binding fragment thereof, peptide or a small molecule directed to Mena.

In any aspect or embodiment of the present disclosure, the agent that inhibits protein expression inhibits DYRK1A expression in the cell.

In any aspect or embodiment of the present disclosure, the agent that promotes protein expression is an agent the results in increased levels of brain derived neurotrophic factor (BDNF) in the cell.

In any aspect or embodiment of the present disclosure, the agent that promotes protein expression is an RNAi agent directed to at least one of HnmpK, PCBP1, or both.

In any aspect or embodiment of the present disclosure, the cell is a neuron.

In any aspect or embodiment of the present disclosure, the administering step results in the modulation of the translation of an mRNA selected from Table 3.

In another aspect, the present disclosure provides a method of ameliorating, treating, or preventing at least one symptom of a disease, disorder, or syndrome associated with the overexpression or accumulation of DYRK1A and/or amyloid precursor protein (APP), the method comprising: providing a subject in need thereof; and administering an effective amount of an agent that inhibits Mena translation, Mena transcription, or the association of Mena with the Mena-RNP complex, wherein the method is effective for ameliorating, treating, or preventing at least one symptom of a disease, disorder, or syndrome associated with the overexpression or accumulation of DYRK1A and/or amyloid precursor protein (APP).

In any aspect or embodiment of the present disclosure, the cell is a neuron.

In any aspect or embodiment of the present disclosure, the disease, disorder, or syndrome is selected from the group consisting of a cognitive disorder, Down Syndrome, Alzheimer's disease, or cancer.

In any aspect or embodiment of the present disclosure, the cancer is a hematological malignancy or brain cancer.

In any aspect or embodiment of the present disclosure, the cancer is breast cancer, pancreatic cancer, lung cancer, or colon cancer.

In any aspect or embodiment of the present disclosure, the agent that inhibits protein expression is selected from an antisense agent, an RNAi agent, an antibody or an antigen binding fragment thereof, or a small molecule directed to Mena.

In a further aspect, the present disclosure provides a method of ameliorating, treating, or preventing at least one symptom of a disease, disorder, or syndrome associated with underexpression of DYRK1A, the method comprising: providing a subject in need thereof; and administering an effective amount of an agent that promotes protein expression by (i) inhibiting the expression of at least one of HnmpK, PCBP1, or both, or (ii) dissociating at least one of HnmpK, PCBP1, or both, from (or preventing the association of at least one of HnmpK, PCBP1, or both with) the Mena-RNP complex in the cell, wherein the method is effective for ameliorating, treating, or preventing at least one symptom of a disease, disorder, or syndrome associated with underexpression of DYRK1A.

In any aspect or embodiment of the present disclosure, the agent that promotes protein expression is an agent the results in increased levels of brain derived neurotrophic factor (BDNF) in the neuron.

In any aspect or embodiment of the present disclosure, the agent that promotes protein expression is an antisense agent or an RNAi agent directed to at least one of HnmpK, PCBP1, or both.

In any aspect or embodiment of the present disclosure, the cell is a neuron.

In any aspect or embodiment of the present disclosure, the subject is selected from the group consisting of a cell, a mammal, and a human.

In yet a further aspect, the present disclosure provides a method of diagnosing a subject as having a Mena-RNP complex associated disease, disorder, or syndrome the method comprising: obtaining or providing a sample from the subject; detecting the expression level of the protein in the sample from the subject; comparing the expression level in the sample to a control having normal expression levels of the protein; and diagnosing the subject as having a disease, disorder, or syndrome associated with the dysregulation of the expression of the protein when the sample has increased or decreased expression relative to the control, wherein the protein is at least one protein selected from Table 3.

In any aspect or embodiment of the present disclosure, the method further comprises administering to the subject an agent that: (a) inhibits protein expression by inhibiting Mena translation, Mena transcription, or the association of Mena with the Mena-RNP complex, or (b) promotes protein expression by: (i) inhibiting the expression of at least one of HnmpK, PCBP1, or both, or (ii) dissociating at least one of HnmpK, PCBP1, or both, from (or preventing the association of at least one of HnmpK, PCBP1, or both with) the Mena-RNP complex in a cell.

In any aspect or embodiment of the present disclosure, the agent that inhibits protein expression is selected from an antisense agent, an RNAi agent, an antibody or an antigen binding fragment thereof, peptide or a small molecule directed to Mena.

In any aspect or embodiment of the present disclosure, the agent that inhibits Mena translation, Mena transcription, or the association of Mena with the Mena-RNP complex inhibits expression of the protein in the cell.

In any aspect or embodiment of the present disclosure, the agent that promotes protein expression is an agent the results in increased levels of brain derived neurotrophic factor (BDNF) in the cell.

In any aspect or embodiment of the present disclosure, the agent that promotes protein expression is an RNAi agent directed to at least one of HnmpK, PCBP1, or both.

In any aspect or embodiment of the present disclosure, the cell is a neuron.

In any aspect or embodiment of the present disclosure, the administering step results in the modulation of the translation of an mRNA selected from Table 3.

In any aspect or embodiment of the present disclosure, detecting the expression level of DYRK1A comprises detecting the protein, which may be accomplished via at least one of immunohistochemistry, enzyme-linked immunosorbent assay, western blot, or a combination thereof.

In any aspect or embodiment of the present disclosure, detecting the expression level of DYRK1A comprises detecting mRNA of the protein, which may be accomplished via at least one of fluorescent in situ hybridization, northern blot, reverse-transcription polymerase chain reaction (RT-PCR), RT real time PCT, microarray, or a combination thereof.

In any aspect or embodiment of the present disclosure, the subject is selected from the group consisting of a cell, a mammal, and a human.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

Akerman, M., David-Eden, H., Pinter, R. Y., Mandel-Gutfreund, Y., 2009. A computational approach for genome-wide mapping of splicing factor binding sites. Genome Biol. 10. doi:10.1186/gb-2009-10-3-r30
Alami, N. H., Smith, R. B., Carrasco, M. a, Williams, L. a, Winborn, C. S., Han, S. S. W., Kiskinis, E., Winborn, B., Freibaum, B. D., Kanagaraj, A., Clare, A. J., Badders, N. M., Bilican, B., Chaum, E., Chandran, S., Shaw, C. E., Eggan, K. C., Maniatis, T., Taylor, J. P., 2014. Axonal transport of TDP-43 mRNA granules is impaired by ALS-causing mutations. Neuron 81, 536-43. doi:10.1016/j.neuron.2013.12.018
Arron, J. R., Winslow, M. M., Polleri, A., Chang, C.-P., Wu, H., Gao, X., Neilson, J. R., Chen, L., Heit, J. J., Kim, S. K., Yamasaki, N., Miyakawa, T., Francke, U., Graef, I. A., Crabtree, G. R., 2006. NFAT dysregulation by increased dosage of DSCR1 and DYRK1A on chromosome 21. Nature 441, 595-600. doi:10.1038/nature04678
Ascano, M., Mukherjee, N., Bandaru, P., Miller, J. B., Nusbaum, J. D., Corcoran, D. L., Langlois, C., Munschauer, M., Dewell, S., Hafner, M., Williams, Z., Ohler, U., Tuschl, T., 2012. FMRP targets distinct mRNA sequence elements to regulate protein expression. Nature 492, 382-6. doi:10.1038/nature11737
Balasanyan, V., Arnold, D. B., 2014. Actin and Myosin-dependent localization of mRNA to dendrites. PLoS One 9, e92349. doi:10.1371/journal.pone.0092349
Baleriola, J., Hengst, U., 2014. Targeting Axonal Protein Synthesis in Neuroregeneration and Degeneration. Neurotherapeutics 57-65. doi:10.1007/s13311-014-0308-8
Barallobre, M. J., Perier, C., Bove, J., Laguna, a, Delabar, J. M., Vila, M., Arbones, M. L., 2014. DYRK1A promotes dopaminergic neuron survival in the developing brain and in a mouse model of Parkinson's disease. Cell Death Dis. 5, e1289. doi:10.1038/cddis.2014.253
Barzik, M., Kotova, T. I., Higgs, H. N., Hazelwood, L., Hanein, D., Gertler, F. B., Schafer, D. A., 2005. Ena/VASP proteins enhance actin polymerization in the presence of barbed end capping proteins. J. Biol. Chem. 280, 28653-28662. doi:10.1074/jbc.M503957200
Bashaw, G. J., Kidd, T., Murray, D., Pawson, T., Goodman, C. S., 2000. Repulsive axon guidance: Abelson and Enabled play opposing roles downstream of the roundabout receptor. Cell 101, 703-715. doi:10.1016/S0092-8674(00)80883-1
Bassell, G. J., Warren, S. T., 2008. Fragile X syndrome: loss of local mRNA regulation alters synaptic development and function. Neuron 60, 201-14. doi:10.1016/j.neuron.2008.10.004
Batista, A. F. R., Hengst, U., 2016. Intra-axonal protein synthesis in development and beyond. Int. J. Dev. Neurosci. doi:10.1016/j.ijdevneu.2016.03.004
Bear, J. E., Gertler, F. B., 2009. Ena/VASP: towards resolving a pointed controversy at the barbed end. J. Cell Sci. 122, 1947-1953. doi:10.1242/jcs.038125
Bear, J. E., Loureiro, J. J., Libova, I., Fa, R., Braunschweig, D.-, 2000. Negative Regulation of Fibroblast Motility by Ena/VASP Proteins 101, 717-728.
Blanco, F. F., Preet, R., Aguado, A., Vishwakarma, V., Stevens, L. E., Vyas, A., Padhye, S., Xu, L., Weir, S. J., Anant, S., Meisner-Kober, N., Brody, J. R., Dixon, D. A., 2016. Impact of HuR inhibition by the small molecule MS-444 on colorectal cancer cell tumorigenesis. Oncotarget 7. doi:10.18632/oncotarget.12189
Bolte, S., Cordelieres, F. P., 2006. A guided tour into subcellular colocalisation analysis in light microscopy. J. Microsc. 224, 13-232. doi:10.1111/j.1365-2818.2006.01706.x
Brown, V., Jin, P., Ceman, S., Darnell, J. C., O'Donnell, W. T., Tenenbaum, S. a, Jin, X., Feng, Y., Wilkinson, K. D., Keene, J. D., Darnell, R. B., Warren, S. T., 2001. Microarray identification of FMRP-associated brain mRNAs and altered mRNA translational profiles in fragile X syndrome. Cell 107, 477-87.
Buxbaum, A. R., Wu, B., Singer, R. H., 2014. Single-Actin mRNA Detection in Neurons Reveals a Mechanism for Regulating Its Translatability. Science (80-.). 343, 419-422. doi:10.1126/science.1242939
Campbell, D. S., Regan, A. G., Lopez, J. S., Tannahill, D., Harris, W. A., Holt, C. E., 2001. Semaphorin 3A Elicits Stage-Dependent Collapse, Turning, and Branching in Xenopus Retinal Growth Cones 21, 8538-8547.
Chen, J.-Y., Lin, J.-R., Tsai, F.-C., Meyer, T., 2013. Dosage of Dyrk1a shifts cells within a p21-cyclin D1 signaling map to control the decision to enter the cell cycle. Mol. Cell 52, 87-100. doi:10.1016/j.molce1.2013.09.009
Coutadeur, S., Benyamine, H., Delalonde, L., de Oliveira, C., Leblond, B., Foucourt, A., Besson, T., Casagrande, A.-S., Taverne, T., Girard, A., Pando, M. P., Desire, L., 2015. A novel DYRK1A (Dual specificity tyrosine phosphorylation-regulated kinase 1A) inhibitor for the treatment of Alzheimer's disease: effect on Tau and amyloid pathologies in vitro. J. Neurochem. 133, 440-51. doi:10.1111/jnc.13018
Darnell, J. C., Van Driesche, S. J., Zhang, C., Hung, K. Y. S., Mele, A., Fraser, C. E., Stone, E. F., Chen, C., Fak, J. J., Chi, S. W., Licatalosi, D. D., Richter, J. D., Darnell, R. B., 2011. FMRP stalls ribosomal translocation on mRNAs linked to synaptic function and autism. Cell 146, 247-61. doi:10.1016/j.cell.2011.06.013
Darnell, R. B., 2010. HITS-CLIP: panoramic views of protein-RNA regulation in living cells. Wiley Interdiscip. Rev. RNA 1, 266-86. doi:10.1002/wrna.31
De Rubeis, S., Bagni, C., 2010. Fragile X mental retardation protein control of neuronal mRNA metabolism: Insights into mRNA stability. Mol. Cell. Neurosci. 43, 43-50. doi:10.1016/j.mcn.2009.09.013
De Rubeis, S., Pasciuto, E., Li, K. W., Fernandez, E., Di Marino, D., Buzzi, A., Ostroff, L. E., Klann, E., Zwartkruis, F. J. T., Komiyama, N. H., Grant, S. G. N., Poujol, C., Choquet, D., Achsel, T., Posthuma, D., Smit, A. B., Bagni, C., 2013. CYFIP1 coordinates mRNA translation and cytoskeleton remodeling to ensure proper dendritic spine formation. Neuron 79, 1169-82. doi:10.1016/j.neuron.2013.06.039
Deglincerti, A., Jaffrey, S. R., 2012. Insights into the roles of local translation from the axonal transcriptome. Open Biol. 2, 1-13. doi:10.1098/rsob.120079
Dent, E. W., Gertler, F. B., 2003. Cytoskeletal dynamics and transport in growth cone motility and guidance. Neuron 40, 209-227. doi:10.1016/S0896-6273(03)00633-0
Dent, E. W., Gupton, S. L., Gertler, F. B., 2011. The Growth Cone Cytoskeleton in Axon outgrowth and guidance.pdf. doi:10.1101/cshperspect.a001800
Dent, E. W., Kwiatkowski, A. V, Mebane, L. M., Philippar, U., Barzik, M., Rubinson, D. a, Gupton, S., Van Veen, J. E., Furman, C., Zhang, J., Alberts, A. S., Mori, S., Gertler, F. B., 2007. Filopodia are required for cortical neurite initiation. Nat. Cell Biol. 9, 1347-59. doi:10.1038/ncb1654
Di Vona, C., Bezdan, D., Islam, A. B. M. M. K., Salichs, E., Lopez-Bigas, N., Ossowski, S., de la Luna, S., 2015. Chromatin-wide profiling of DYRK1A reveals a role as a gene-specific RNA polymerase II CTD kinase. Mol. Cell 57, 506-20. doi:10.1016/j.molcel.2014.12.026
Drees, F., Gertler, F. B., 2008. Ena/VASP: proteins at the tip of the nervous system. Curr. Opin. Neurobiol. 18, 53-59. doi:10.1016/j.conb.2008.05.007
Folci, A., Mapelli, L., Sassone, J., Prestori, F., Angelo, E. D., Bassani, S., Passafaro, M., 2014. Loss of hnRNP K Impairs Synaptic Plasticity in Hippocampal Neurons 34, 9088-9095. doi:10.1523/JNEUROSCI.0303-14.2014
Fritzsche, R., Karra, D., Bennett, K. L., Ang, F. Y., Heraud-Farlow, J. E., Tolino, M., Doyle, M., Bauer, K. E., Thomas, S., Planyaysky, M., Am, E., Bakosova, A., Jungwirth, K., Hormann, A., Palfi, Z., Sandholzer, J., Schwarz, M., Macchi, P., Colinge, J., Superti-Furga, G., Kiebler, M. a, 2013. Interactome of two diverse RNA granules links mRNA localization to translational repression in neurons. Cell Rep. 5, 1749-62. doi:10.1016/j.celrep.2013.11.023
Gebauer, F., Hentze, M. W., 2004. MOLECULAR MECHANISMS OF TRANSLATIONAL CONTROL 5. doi:10.1038/nrm1488
Gertler, F., Condeelis, J., 2011. Metastasis: tumor cells becoming MENAcing. Trends Cell Biol. 21, 81-90. doi:10.1016/j.tcb.2010.10.001
Gertler, F. B., Niebuhr, K., Reinhard, M., Wehland, J., Soriano, P., 1996. Mena, a relative of VASP and Drosophila enabled, is implicated in the control of microfilament dynamics. Cell 87, 227-239. doi:10.1016/50092-8674(00)81341-0
Giesemann, T., Nawrotzki, R., Berho, K., Rothkegel, M., Schlu, K., Schrader, N., Schindelin, H., Mendel, R. R., Kirsch, J., Jockusch, B. M., 2003. Complex Formation between the Postsynaptic Scaffolding Protein Gephyrin, Profilin, and Mena: A Possible Link to the Microfilament System 23, 8330-8339.
Goh, K. L., Cai, L., Cepko, C. L., Gertler, F. B., 2002. Ena/VASP Proteins Regulate Cortical Neuronal Positioning. Curr. Biol. 12, 565-569. doi:10.1016/50960-9822(02)00725-X
Gupton, S. L., Gertler, F. B., 2010. Integrin signaling switches the cytoskeletal and exocytic machinery that drives neuritogenesis. Dev. Cell 18, 725-736. doi:10.1016/j.devcel.2010.02.017
Gupton, S. L., Riquelme, D., Hughes-Alford, S. K., Tadros, J., Rudina, S. S., Hynes, R. O., Lauffenburger, D., Gertler, F. B., 2012. Mena binds alpha5 integrin directly and modulates alpha5beta1 function. J. Cell Biol. 198, 657-676. doi:10.1083/jcb.201202079
Hammerle, B., Carnicero, a., Elizalde, C., Ceron, J., Martinez, S., Tejedor, F. J., 2003. Expression patterns and subcellular localization of the Down syndrome candidate protein MNB/DYRK1A suggest a role in late neuronal differentiation. Eur. J. Neurosci. 17, 2277-2286. doi:10.1046/j.1460-9568.2003.02665.x
Hammerle, B., Elizalde, C., Tejedor, F. J., 2008. The spatio-temporal and subcellular expression of the candidate Down syndrome gene Mnb/Dyrk1A in the developing mouse brain suggests distinct sequential roles in neuronal development. Eur. J. Neurosci. 27, 1061-74. doi:10.1111/j.1460-9568.2008.06092.x
Holland, J. D., Klaus, A., Garratt, A. N., Birchmeier, W., 2013. Wnt signaling in stem and cancer stem cells. Curr. Opin. Cell Biol. 25, 254-264. doi:10.1016/j.ceb.2013.01.004
Hutten, S., Sharangdhar, T., Kiebler, M., 2014. Unmasking the messenger. RNA Biol. 11, 992-7. doi:10.4161/rna.32091
Janel, N., Sarazin, M., Corlier, F., Come, H., de Souza, L. C., Hamelin, L., Aka, a, Lagarde, J., Blehaut, H., Hindie, V., Rain, J.-C., Arbones, M. L., Dubois, B., Potier, M. C., Bottlaender, M., Delabar, J. M., 2014. Plasma DYRK1A as a novel risk factor for Alzheimer's disease. Transl. Psychiatry 4, e425. doi:10.1038/tp.2014.61
Jung, H., Holt, C. E., 2011. Local translation of mRNAs in neural development. Wiley Interdiscip. Rev. RNA 2, 153-65. doi:10.1002/wrna.53
Jung, H., Yoon, B. C., Holt, C. E., 2012. Axonal mRNA localization and local protein synthesis in nervous system assembly, maintenance and repair. Nat. Rev. Neurosci. 13, 308-24. doi:10.1038/nrn3210
Kim, E., Jung, H., 2015. Local protein synthesis in neuronal axons: why and how we study. BMB Rep 48, 139-146. doi:10.5483/BMBRep.2015.48.3.010
Kim, H., Lee, K.-S., Kim, A.-K., Choi, M., Choi, K., Kang, M., Chi, S.-W., Lee, M.-S., Lee, J.-S., Lee, S.-Y., Song, W.-J., Yu, K., Cho, S., 2016. A chemical with proven clinical safety rescues Down-syndrome-related phenotypes in through DYRK1A inhibition. Dis. Model. Mech. 9, 839-48. doi:10.1242/dmm.025668
Kindler, S., Buzzi, A., Marino, D. Di, 2012. Synaptic Plasticity. Advances in Experimental Medicine and Biology 970, 285-305. doi:10.1007/978-3-7091-0932-8
Krause, M., Dent, E. W., Bear, J. E., Loureiro, J. J., Gertler, F. B., 2003. E NA/VASP P ROTEINS: Regulators of the Actin Cytoskeleton and Cell Migration. Annu. Rev. Cell Dev. Biol. 19, 541-564. doi:10.1146/annurev.cellbio.19.050103.103356
Krumm, N., O'Roak, B. J., Shendure, J., Eichler, E. E., 2014. A de novo convergence of autism genetics and molecular neuroscience. Trends Neurosci. 37, 95-105. doi:10.1016/j.tins.2013.11.005
Kwiatkowski, A. V, Rubinson, D. a, Dent, E. W., Edward van Veen, J., Leslie, J. D., Zhang, J., Mebane, L. M., Philippar, U., Pinheiro, E. M., Burds, A. a, Bronson, R. T., Mori, S., Fassler, R., Gertler, F. B., 2007. Ena/VASP Is Required for neuritogenesis in the developing cortex. Neuron 56, 441-55. doi:10.1016/j.neuron.2007.09.008
Lambert, N., Robertson, A., Jangi, M., McGeary, S., Sharp, P. A., Burge, C. B., 2014. RNA Bind-n-Seq: Quantitative Assessment of the Sequence and Structural Binding Specificity of RNA Binding Proteins. Mol. Cell 54, 887-900. doi:10.1016/j.molcel.2014.04.016
Lanier, L. M., Gates, M. A., Witke, W., Menzies, A. S., Wehman, A. M., Macklis, J. D., Kwiatkowski, D., Soriano, P., Gertler, F. B., 1999. Mena is required for neurulation and commissure formation. Neuron 22, 313-325. doi:10.1016/S0896-6273(00)81092-2
Lanier, L. M., Gertler, F. B., 2000. Actin cytoskeleton: Thinking globally, actin' locally. Curr. Biol. 10. doi:10.1016/S0960-9822(00)00685-0
Lebrand, C., Dent, E. W., Strasser, G. A., Lanier, L. M., Krause, M., Svitkina, T. M., Borisy, G. G., Gertler, F. B., 2004. Critical role of Ena/VASP proteins for filopodia formation in neurons and in function downstream of netrin-1. Neuron 42, 37-49. doi:10.1016/S0896-6273(04)00108-4
Li, J., Yu, B., Deng, P., Cheng, Y., Yu, Y., Kevork, K., Ramadoss, S., Ding, X., Li, X., Wang, C.-Y., 2017. KDM3 epigenetically controls tumorigenic potentials of human colorectal cancer stem cells through Wnt/β-catenin signalling. Nat. Commun. 8, 15146. doi:10.1038/ncomms15146
Li, W., Li, Y., Gao, F.-B., 2005. Abelson, enabled, and p120 catenin exert distinct effects on dendritic morphogenesis in Drosophila. Dev. Dyn. 234, 512-22. doi:10.1002/dvdy.20496
Licatalosi, D. D., Mele, A., Fak, J. J., Ule, J., Kayikci, M., Chi, S. W., Clark, T. a, Schweitzer, A. C., Blume, J. E., Wang, X., Darnell, J. C., Darnell, R. B., 2008. HITS-CLIP yields genome-wide insights into brain alternative RNA processing. Nature 456, 464-9. doi:10.1038/nature07488
Lin, Y.-L., Lei, Y.-T., Hong, C.-J., Hsueh, Y.-P., 2007. Syndecan-2 induces filopodia and dendritic spine formation via the neurofibromin-PKA-Ena/VASP pathway. J. Cell Biol. 177, 829-41. doi:10.1083/jcb.200608121
Lu, L., Zheng, L., Si, Y., Luo, W., Dujardin, G., Kwan, T., Potochick, N. R., Thompson, S. R., Schneider, D. A., King, P. H., 2014. Hu antigen R (HuR) is a positive regulator of the RNA-binding proteins TDP-43 and FUS/TLS: Implications for amyotrophic lateral sclerosis. J. Biol. Chem. 289, 31792-31804. doi:10.1074/jbc.M114.573246
Malinge, S., Bliss-Moreau, M., Kirsammer, G., Diebold, L., Chlon, T., Gurbuxani, S., Crispino, J. D., 2012. Increased dosage of the chromosome 21 ortholog Dyrk1a promotes megakaryoblastic leukemia in a murine model of down syndrome. J. Clin. Invest. 122, 948-962. doi:10.1172/JC160455
McCandless, D. W., 2012. Epilepsy and Autism 1-18. doi:10.1007/978-1-4614-0361-6
McConnell, R. E., Van Veen, J. E., Vidaki, M., Kwiatkowski, A. V., Meyer, A. S., Gertler, F. B., 2016. A requirement for filopodia extension toward Slit during Robo-mediated axon repulsion. J. Cell Biol. 213, 261-274. doi:10.1083/jcb.201509062
Menzies, A. S., Aszodi, A., Williams, S. E., Pfeifer, A., Wehman, A. M., Goh, K. L., Mason, C. A., Fassler, R., Gertler, F. B., 2004. Mena and Vasodilator-Stimulated Phosphoprotein Are Required for Multiple Actin-Dependent Processes That Shape the Vertebrate Nervous System. J. Neurosci. 24, 8029-8038. doi:10.1523/JNEUROSCI.1057-04.2004
Mili, S., Moissoglu, K., Macara, I. G., 2008. Genome-wide screen reveals APC-associated RNAs enriched in cell protrusions. Nature 453, 115-9. doi:10.1038/nature06888
Najafov, A., Seker, T., Even, I., Hoxhaj, G., Selvi, O., Ozel, D. E., Koman, A., Birgül-İyison, N., 2012. MENA is a transcriptional target of the Wnt/beta-catenin pathway. PLoS One 7, e37013. doi:10.1371/journal.pone.0037013
Niebuhr, K., Ebel, F., Frank, R., Reinhard, M., Domann, E., Carl, U. D., Walter, U., Gertler, F. B., Wehland, J., Chakraborty, T., 1997. A novel proline-rich motif present in ActA of Listeria monocytogenes and cytoskeletal proteins is the ligand for the EVH1 domain, a protein module present in the Ena/VASP family. EMBO J. 16, 5433-5444. doi:10.1093/emboj/16.17.5433
O'Roak, B. J., Vives, L., Fu, W., Egertson, J. D., Stanaway, I. B., Phelps, I. G., Carvill, G., Kumar, A., Lee, C., Ankenman, K., Munson, J., Hiatt, J. B., Turner, E. H., Levy, R., O'Day, D. R., Krumm, N., Coe, B. P., Martin, B. K., Borenstein, E., Nickerson, D. a, Mefford, H. C., Doherty, D., Akey, J. M., Bernier, R., Eichler, E. E., Shendure, J., 2012. Multiplex targeted sequencing identifies recurrently mutated genes in autism spectrum disorders. Science 338, 1619-22. doi:10.1126/science.1227764
Ortiz-Abalia, J., Sahun, I., Altafaj, X., Andreu, N., Estivill, X., Dierssen, M., Fillat, C., 2008. Targeting Dyrk1A with AAVshRNA Attenuates Motor Alterations in TgDyrk1A, a Mouse Model of Down Syndrome. Am. J. Hum. Genet. 83, 479-488. doi:10.1016/j.ajhg.2008.09.010
Ostareck-lederer, A., Ostareck, D. H., Cans, C., Neubauer, G., Bomsztyk, K., Superti-furga, G., Hentze, M. W., 2002. c-Src-Mediated Phosphorylation of hnRNP K Drives Translational Activation of Specifically Silenced mRNAs 22, 4535-4543. doi:10.1128/MCB .22.13.4535
Paz, I., Akerman, M., Dror, I., Kosti, I., Mandel-Gutfreund, Y., 2010. SFmap: a web server for motif analysis and prediction of splicing factor binding sites. Nucleic Acids Res. 38, W281-W285. doi:10.1093/nar/gkq444
Preitner, N., Quan, J., Nowakowski, D. W., Hancock, M. L., Shi, J., Tcherkezian, J., Young-Pearse, T. L., Flanagan, J. G., 2014. APC is an RNA-binding protein, and its interactome provides a link to neural development and microtubule assembly. Cell 158, 368-382. doi:10.1016/j.cell.2014.05.042
Proepper, C., Steinestel, K., Schmeisser, M. J., Heinrich, J., Steinestel, J., Bockmann, J., Liebau, S., Boeckers, T. M., 2011. Heterogeneous Nuclear Ribonucleoprotein K Interacts with Abi-1 at Postsynaptic Sites and Modulates Dendritic Spine Morphology 6. doi:10.1371/journal.pone.0027045
Qian, W., Jin, N., Shi, J., Yin, X., Jin, X., Wang, S., Cao, M., Iqbal, K., Gong, C.-X., Liu, F., 2013. Dual-specificity tyrosine phosphorylation-regulated kinase 1A (Dyrk1A) enhances tau expression. J. Alzheimers. Dis. 37, 529-38. doi:10.3233/JAD-130824
Rachdi, L., Kariyawasam, D., Mello, V., Herault, Y., Janel, N., Delabar, J. M., Polak, M., Scharfmann, R., 2014. Dyrk1A induces pancreatic?? cell mass expansion and improves glucose tolerance. Cell Cycle 13, 2221-2229. doi:10.4161/cc.29250
Sahin, M., Sur, M., 2015. Genes, circuits, and precision therapies for autism and related neurodevelopmental disorders. Science (80-.). 350, aab3897-. doi:10.1126/science.aab3897
Sanchez-Gomez, P. F.-M. C. Z. P., 2015. DYRK1A: the double-edged kinase as a protagonist in cell growth and tumorigenesis [WWW Document]. Mol. Cell. Oncol. URL http://www.tandfonline.com/doi/pdf/10.4161/23723548.2014.970048 (accessed May 12, 2016).
Santos, A. R., Comprido, D., Duarte, C. B., 2010. Regulation of local translation at the synapse by BDNF. Prog. Neurobiol. 92, 505-516. doi:10.1016/j.pneurobio.2010.08.004
Schratt, G. M., Nigh, E. a, Chen, W. G., Hu, L., Greenberg, M. E., 2004. BDNF regulates the translation of a select group of mRNAs by a mammalian target of rapamycin-phosphatidylinositol 3-kinase-dependent pathway during neuronal development. J. Neurosci. 24, 7366-77. doi:10.1523/JNEUROSCI.1739-04.2004
Szostak, E., Gebauer, F., 2013. Translational control by 3′-UTR-binding proteins. Brief. Funct. Genomics 12, 58-65. doi:10.1093/bfgp/els056
Tejedor, F. J., Hämmerle, B., 2011. MNB/DYRK1A as a multiple regulator of neuronal development. FEBS J. 278, 223-35. doi:10.1111/j.1742-4658.2010.07954.x
Thiele, B., Doller, A., Ka, T., Pregla, R., Hetzer, R., Regitz-zagrosek, V., 2016. RNA-Binding Proteins Heterogeneous Nuclear Ribonucleoprotein A1, E1, and K Are Involved in Post-Transcriptional Control of Collagen I and III Synthesis. doi:10.1161/01.RES.0000149166.33833.08
Torvund-jensen, J., Steengaard, J., Reimer, L., Fihl, L. B., Laursen, L. S., 2014. Transport and translation of MBP mRNA is regulated differently by distinct hnRNP proteins 2, 1550-1564. doi:10.1242/jcs.140855
Townson, S. M., Dobrzycka, K. M., Lee, A. V., Air, M., Deng, W., Kang, K., Jiang, S., Kioka, N., Michaelis, K., Oesterreich, S., 2003. SAFB2, a new scaffold attachment factor homolog and estrogen receptor corepressor. J. Biol. Chem. 278, 20059-20068. doi:10.1074/jbc.M212988200
van Bon, B. W. M., Coe, B. P., Bernier, R., Green, C., Gerdts, J., Witherspoon, K., Kleefstra, T., Willemsen, M. H., Kumar, R., Bosco, P., Fichera, M., Li, D., Amaral, D., Cristofoli, F., Peeters, H., Haan, E., Romano, C., Mefford, H. C., Scheffer, I., Gecz, J., de Vries, B. B. a, Eichler, E. E., 2015. Disruptive de novo mutations of DYRK1A lead to a syndromic form of autism and ID. Mol. Psychiatry 1-7. doi:10.1038/mp.2015.5
Van Bon, B. W. M., Hoischen, A., Hehir-Kwa, J., De Brouwer, A. P. M., Ruivenkamp, C., Gijsbers, A. C. J., Marcelis, C. L., De Leeuw, N., Veltman, J. A., Brunner, H. G., De Vries, B. B. A., 2011. Intragenic deletion in DYRK1A leads to mental retardation and primary microcephaly. Clin. Genet. 79, 296-299. doi:10.1111/j.1399-0004.2010.01544.x
Verma, P., Chierzi, S., Codd, A. M., Campbell, D. S., Meyer, R. L., Holt, C. E., Fawcett, J. W., 2005. Axonal protein synthesis and degradation are necessary for efficient growth cone regeneration. J. Neurosci. 25, 331-42. doi:10.1523/JNEUROSCI.3073-04.2005
Wang, H., Ding, N., Guo, J., Xia, J., Ruan, Y., 2016. Dysregulation of TTP and HuR plays an important role in cancers. Tumor Biol. 37, 14451-14461. doi:10.1007/s13277-016-5397-z
Wegiel, J. J., Kuchna, I., Nowicki, K., Imaki, H., Marchi, E., Ma, S. Y., Chauhan, A., Chauhan, V., Bobrowicz, T. W., de Leon, M., Louis, L. a Saint, Cohen, I. L., London, E., Brown, W. T., Wisniewski, T., 2010. The neuropathology of autism: defects of neurogenesis and neuronal migration, and dysplastic changes. Acta Neuropathol. 119, 755-70. doi:10.1007/s00401-010-0655-4
Yu, T. W., Hao, J. C., Lim, W., Tessier-Lavigne, M., Bargmann, C. I., 2002. Shared receptors in axon guidance: SAX-3/Robo signals via UNC-34/Enabled and a Netrin-independent UNC-40/DCC function. Nat. Neurosci. 5, 1147-54. doi:10.1038/nn956

What is claimed is:

1.-28. (canceled)

29. A method of treating a dual specificity tyrosine-phosphorylation-regulated kinase 1A (DYRK1A)-related or an amyloid precursor protein (APP)-related pathological condition in a subject comprising the steps of:
providing a subject suffering from overexpression or accumulation of DYRK1A or APP; and
administering an effective amount of an agent that inhibits at least one of Mena translation, Mena transcription, or the association of Mena with the Mena-ribonucleoprotein (RNP) complex, wherein the method is effective for treating or ameliorating at least one symptom of the DYRK1A-related pathological condition, the APP-related pathological condition or both.

30. The method of claim 29, wherein the subject is selected from the group of a cell, a mammal, and a human.

31. The method of claim 30, wherein the cell is a neuron.

32. The method of claim 29, wherein the DYRK1A-related pathological condition is selected from the group consisting of a cognitive disorder, Down Syndrome, and cancer.

33. The method of claim 29, wherein the APP-related pathological condition is selected from the group consisting of a cognitive disorder, and Alzheimer's disease.

34. The method of claim 32, wherein the cancer is a hematological malignancy, brain cancer, breast cancer, pancreatic cancer, lung cancer, or colon cancer.

35. The method of claim 29, wherein the agent that inhibits protein expression is selected from a Mena antisense nucleic acid, a Mena inhibitory RNA, an anti-Mena antibody or an antigen binding fragment thereof, or a small molecule inhibitor of Mena.

36. A method treating a tyrosine-phosphorylation-regulated kinase 1A (DYRK1A)-related pathological condition comprising the steps of:
providing a subject suffering from underexpression of DYRK1A; and
administering an effective amount of an agent that promotes DYRK1A protein expression by:
(i) inhibiting the expression of at least one of heterogeneous nuclear ribonucleoprotein K (HnrnpK), poly(rC)-binding protein 1 (PCBP1), or combination thereof,
(ii) dissociating at least one of HnmpK, PCBP1, or combination thereof, from a Mena-ribonucleoprotein (RNP) complex, or
(iii) preventing the association of at least one of HnmpK, PCBP1, or combination thereof, with the Mena-RNP complex,
wherein the method is effective for treating or ameliorating at least one symptom of a DYRK1A-related pathological condition associated with underexpression of DYRK1A.

37. The method of claim 36, wherein the agent that promotes DYRK1A protein expression is an agent the results in increased levels of brain derived neurotrophic factor (BDNF) in the neuron.

38. The method of claim 36, wherein the agent that promotes DYRK1A protein expression is an antisense agent or an RNAi agent directed to at least one of HnmpK, PCBP1, or combination thereof.

39. The method of claim 36, wherein the subject is selected from the group consisting of a cell, a mammal, and a human.

40. The method of claim 39, wherein the cell is a neuron.

41. A method of treating a subject having a Mena-ribonucleoprotein (RNP) complex associated pathological condition, the method comprising the steps of:
administering to the subject in need thereof an effective amount of an agent that:
(i) inhibits protein expression by inhibiting Mena translation, Mena transcription, or the association of Mena with the Mena-RNP complex, or
(ii) promotes protein expression by:
a. inhibiting the expression of at least one of HnmpK, PCBP1 or combination thereof,
b. dissociating at least one of HnmpK, PCBP1 or combination thereof, from the Mena-RNP complex; or
c. preventing the association of at least one of HnmpK, PCBP1 or combination thereof, with the Mena-RNP complex,
wherein the method is effective to treat or ameliorate at least one symptom of the Mena-ribonucleoprotein (RNP) complex associated pathological condition.

42. The method of claim 41, wherein the subject is selected from the group consisting of a cell, a mammal, and a human.

43. The method of claim 42, wherein the cell is a neuron.

44. The method of claim 41, wherein the agent that inhibits protein expression is selected from a Mena antisense nucleic acid, a Mena inhibitory RNA, an anti-Mena antibody or an antigen binding fragment thereof, or a small molecule inhibitor of Mena.
